(19) United States
(12) Patent Application Publication
(10) Pub. No.: US 2018/0250313 A1
(43) Pub. Date: Sep. 6, 2018

(54) COMPOUNDS AND FORMULATIONS FOR TREATING OPHTHALMIC DISEASES

(71) Applicant: VIEWPOINT THERAPEUTICS, INC., San Francisco, CA (US)

(72) Inventors: Leah MAKLEY, San Francisco, CA (US); Angel PADILLA, Aliso Viejo, CA (US); David BAKER, Carlsbad, CA (US); Gary COOK, Westford, MA (US); Harun TAKRURI, Newport Beach, CA (US); Jery CAGLE, Fort Worth, TX (US); Emmett CUMMINGHAM, Hillsborough, CA (US)

(21) Appl. No.: 15/758,705

(22) PCT Filed: Sep. 8, 2016
(86) PCT No.: PCT/US16/50823

§ 371 Date: Mar. 8, 2018

Related U.S. Application Data
(60) Provisional application No. 62/215,629, filed on Sep. 8, 2015.
(60) Provisional application No. 62/269,013, filed on Dec. 17, 2015.
(60) Provisional application No. 62/269,019, filed on Dec. 17, 2015.

(51) Int. Cl. A61K 31/575; A61K 9/00; A61P 27/12; A61K 47/02; A61K 47/38

(52) U.S. Cl. CPC A61K 31/575; A61K 9/0019; A61K 47/38; A61P 27/12; A61K 47/02; A61K 9/0048

(57) ABSTRACT

The present disclosure is directed to compositions, formulations and methods of use thereof in the treatment and prevention of ocular conditions including cataract and presbyopia.

### CROSS-REFERENCE TO RELATED APPLICATIONS

This application claims the priority of U.S. Provisional Patent Application No. 62/215,629, filed Sep. 8, 2015, the entire contents of which are incorporated herein by reference.

### BACKGROUND OF THE INVENTION

Cataract affects more than 24 million Americans age 40 and older and by age 75, half of all Americans have cataract. Cataract is a clouding of the lens in the eye that affects vision. The conventional treatment for cataract is surgical replacement with an artificial intraocular lens. Surgical treatment of cataract, however, is costly and an artificial lens does not have the same overall optical qualities as a normal lens.

It is estimated that approximately 112 million Americans currently suffer from presbyopia. Presbyopia is age-related far-sightedness that commonly manifests begins between the ages of 40 and 50, initially causing blurred vision, difficulty seeing in dim light, and eye strain. In healthy eyes, the lens is able to focus light from objects at different distances by a process called accommodation—a slight change in lens shape by the surrounding muscles to change the way light passes through the interior of the lens and onto the retina where the image is formed. During accommodation, muscles surrounding the lens contract, causing the lens to change shape and increasing the focusing power of the eye. This allows focus and clear vision at near and far distances. With increasing age, the lens becomes stiffer as its structural crystallin proteins become misfolded. This increased lens stiffness limits the eye's ability to focus for reading or other tasks that require clear vision at near distances. Reading glasses or glasses with progressive lenses are the most common correction for presbyopia although surgical options are available as well.

As cataract and presbyopia affect billions of people worldwide, there exists a significant need for new methods for treating and preventing these diseases.

### SUMMARY OF THE INVENTION

In certain aspects, the disclosure provides a pharmaceutical formulation comprising from about 0.05 wt % to about 5 wt % of a compound represented by formula (IIIC):

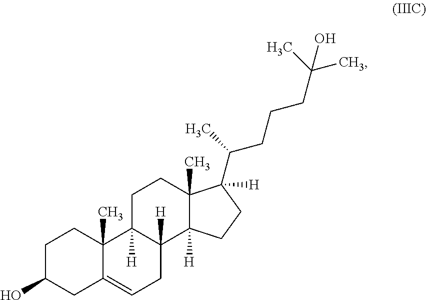

or a salt thereof, and one or more pharmaceutically acceptable excipients. In certain embodiments, the formulation of the disclosure comprises from about 0.1 wt % to about 4 wt %, about 0.5 wt % to about 4 wt %, or about 2 wt % to about 4 wt % of a compound or salt of formula (IIIC).

In certain embodiments, the pharmaceutical formulation comprises the compound or salt of formula (IIIC) is in the form of particles and wherein the particles have an average largest diameter selected from about 1 nm to about 1 μm. The particles may have an average diameter selected from about 1 nm to about 200 nm, about 400 nm to about 600 nm, or about 450 to about 550 nm. In certain embodiments, greater than 80% of the particles in the formulation have an average largest diameter selected from about 450 nm to about 550 nm.

In certain embodiments, the pharmaceutical formulation is aqueous, such as the formulation comprises at least about 90 wt % water.

In certain embodiments, the pharmaceutical formulation comprises an agent that increases the viscosity of the formulation. Agents that increase the viscosity of the formulation may be selected from carboxymethyl cellulose (CMC), hydroxyethyl cellulose, polyethylene glycol (PEG), sodium carboxymethyl cellulose, hydroxypropyl methyl cellulose (HPMC), sorbitol, gellan gum (high or low acyl), xanthan gum, dextran, guar gum, locust bean gum, sodium alginate, agar, gelatin, chitosan, pectin, alginates, xyloglucan, polyvinyl alcohol, polyvinyl pyrrolidone, carrageenan and combinations thereof. In certain embodiments, the agent that increases the viscosity of the formulation is gellan gum. In certain embodiments, the pharmaceutical formulation has a viscosity of about 0.005 Pa·s to about 0.030 Pa·s.

In certain embodiments, the pharmaceutical formulation comprises an agent for adjusting the pH of the formulation. The agent for adjusting the pH of the formulation may be selected from hydrochloric acid, boric acid, sodium hydroxide and potassium hydroxide. The agent for adjusting the pH of the formulation may be boric acid. In certain embodiments, the formulation has a pH selected from about 5 to about 9, about 7 to about 8, such as about 7.4.

In certain embodiments, the formulation comprises an agent for adjusting the osmolarity of the formulation. The agent for adjusting the osmolarity of the formulation may be selected from mannitol, sodium chloride, sodium nitrate, sodium sulfate, dextrose, potassium chloride, glycerin, propylene glycol, calcium chloride, and magnesium chloride. In certain embodiments, the agent for adjusting the osmolarity of the formulation is mannitol.

In certain embodiments, the formulation comprises a buffering agent. The buffering agent may be selected from tromethamine, potassium phosphate, sodium phosphate, saline sodium citrate buffer (SSC), acetate, saline, physiological saline, phosphate buffer saline (PBS), 4-2-hydroxyethyl-1-piperazineethanesulfonic acid buffer (HEPES), 3-(N-morpholino)propanesulfonic acid buffer (MOPS), and piperazine-N,N′-bis(2-ethanesulfonic acid) buffer (PIPES), sodium acetate-boric acid stock solution, boric acid-sodium carbonate with sodium chloride solution, boric acid-sodium borate buffer, sodium and potassium phosphate buffers, boric acid-sodium carbonate with potassium chloride, or combinations thereof. In certain embodiments, the buffering agent is tromethamine. The pharmaceutical formulation may comprise from about 0.1 wt % to about 4 wt % of a buffering agent.

The pharmaceutical formulation may comprise a dispersion agent. Examples of dispersion agents include surfactants such as sorbitan ether esters of oleic acid, polysorbate-80, and polysorbate-20, cationic surfactants, and anionic surfactants. In certain embodiments, the pharmaceutical formulation comprises from about 0.01 wt % to about 1 wt % of a dispersion agent, e.g., polysorbate-80.

In certain embodiments, the formulation comprises a preservative agent. The preservative agent may be selected from benzalkonium chloride, ethylenediaminetetraacetic acid (EDTA), chlorobutanol, phenylmercuric acetate, phenylmercuric nitrate, chlorhexidine acetate, thimerosal, and benzethonium chloride. In certain embodiments, the formulation comprises from about 0.001 wt % to about 0.1 wt % of a preservative agent. In other embodiments, the formulation does not include a preservative agent.

In certain aspects, the disclosure provides a method for treating an ophthalmic disease comprising administering a pharmaceutical formulation described herein to the eye of a subject in need thereof. In certain embodiments, the pharmaceutical formulation is administered topically, by intravitreal injection or intracameral injection. In certain embodiments, the pharmaceutical formulation is administered by intravitreal injection or intracameral injection. In certain embodiments, a pharmaceutical formulation for intravitreal injection is administered in one or more doses wherein each dose is selected from about 60 μL to about 120 μL or about 80 μL to about 110 μL.

In certain embodiments, a dose of the pharmaceutical formulation is administered once monthly, once every six weeks, once every two months, once every six months, or once yearly. A dose of the pharmaceutical formulation may be administered once a month for three consecutive months followed by a dosing holiday of one month, two months, three months, four months, five months, six months, nine months or a year. A dose of the pharmaceutical formulation may be administered once a month for two consecutive months followed by a dosing holiday of one month, two months, three months, four months, five months, six months, nine months or a year.

In certain embodiments, the pharmaceutical formulation is administered topically.

In certain embodiments, a pharmaceutical composition of the disclosure is used to treat or prevent an ophthalmic disease such as cataract or presbyopia.

In certain aspects, the disclosure provides a method of treating or preventing a near vision disorder of a subject, comprising administering to a subject in need thereof a compound of Formula (III):

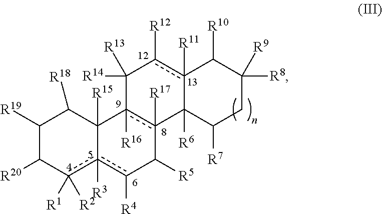

or a salt thereof, wherein:

R¹, R², R³, R⁴, R⁶, R⁸, R⁹, R¹¹, R¹², R¹³, R¹⁴, R¹⁵, R¹⁶, and R¹⁷ are independently selected from hydrogen, halogen, —OR³⁰, —SR³⁰, —OSO₃R³⁰, —OPO₃R³⁰, —N(R³¹)₂, —C(O)R³⁰, —C(O)OR³⁰, —OC(O)R³⁰, —NO₂, —CN, optionally substituted C₁-C₁₀ alkyl, optionally substituted C₂-C₁₀ alkenyl, optionally substituted C₂-C₁₀ alkynyl, optionally substituted carbocycle and optionally substituted heterocycle; R¹ taken together with R² is further selected from ═O, ═S, and ═N(R³¹); R⁸ taken together with R⁹ is further selected from ═O, ═S, and ═N(R³¹); R¹³ taken together with R¹⁴ is further selected from ═O, ═S, and ═N(R³¹); R⁹ and R¹⁰ taken together with the atoms to which they are attached may further form an optionally substituted carbocycle or optionally substituted heterocycle; and wherein R³ is absent when there is a double bond between carbons 5 and 6, R¹⁶ and R¹⁷ are absent when there is a double bond between carbons 8 and 9, R¹¹ is absent when there is a double bond between carbons 12 and 13; and R² and R³ are absent and there is a single bond between carbons 5 and 6 when there is a double bond between carbons 4 and 5;

R⁵, R⁷, R¹⁰, R¹⁸, R¹⁹ and R²⁰ are independently selected from hydrogen, halogen, —OR³⁰, —SR³⁰, —OSO₃R³⁰, —OPO₃R³⁰, —N(R³¹)₂, —C(O)R³⁰, —C(O)OR³⁰, —OC(O)R³⁰, —NO₂, —CN, ═O, ═S, ═N(R³¹), optionally substituted C₁-C₁₀ alkyl, optionally substituted C₂-C₁₀ alkenyl, C₂-C₁₀ alkynyl, optionally substituted carbocycle and optionally substituted heterocycle; each R³¹ is independently selected from hydrogen, —OR³⁰, —SR³⁰, —S(O)R³⁰, —S(O)₂R³⁰, —C(O)R³⁰, —C(O)OR³⁰, optionally substituted C₁-C₁₀ alkyl, optionally substituted C₂-C₁₀ alkenyl, C₂-C₁₀ alkynyl, optionally substituted carbocycle and optionally substituted heterocycle;

each R³⁰ is independently selected from hydrogen, optionally substituted C₁-C₆ alkyl, optionally substituted C₂-C₆ alkenyl, optionally substituted C₂-C₆ alkynyl, optionally substituted carbocycle and optionally substituted heterocycle; and

n is selected from 0 or 1,

wherein the near vision disorder is not cataract.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The invention has other advantages and features which will be more readily apparent from the following detailed description of the invention and the appended claims, when taken in conjunction with the accompanying drawings, in which:

FIG. 1 depicts a microfluidics system.

FIG. 2 illustrates how particle size decreases with increased passes through a microfluidics system.

FIG. 3 illustrates how particle size differs before and after processing in a microfluidics system.

FIG. 4 displays the results of experiments determining the lens exposure of compounds of the invention.

FIG. 5 displays the results of experiments determining the corneal exposure of compounds of the invention.

FIG. 6 displays the results of experiments determining the retinal exposure of compounds of the invention.

FIG. 7 displays the results of experiments determining the ciliary body exposure of compounds of the invention.

FIG. 8 displays the results of experiments determining the exposure of compounds of the invention in various tissues of human globes.

FIG. 9 displays the results of experiments determining the kinetics of exposure of compounds of the invention in the lens of human globes.

FIG. 10 depicts the concentration of 25-hydroxycholesterol in the vitreous humor at 3 wt % and 0.5 wt % following intravitreal administration in rabbits, i.e., 2 hr, 24 hr, and 168 hr following administration.

FIG. 11 depicts the concentration of 25-hydroxycholesterol in the lens at 3 wt % and 0.5 wt % following intravitreal administration in rabbits, i.e., 2 hr, 24 hr, and 168 hr following administration.

FIG. 12 depicts a Snellen vision chart.

FIG. 13 depicts a Jaeger vision chart.

FIG. 14 depicts a LogMAR chart.

### DETAILED DESCRIPTION OF THE INVENTION

### Definitions

Unless defined otherwise, all technical and scientific terms used herein have the same meaning as is commonly understood by one of skill in the art to which this invention belongs. All patents and publications referred to herein are incorporated by reference.

As used in the specification and claims, the singular form “a”, “an” and “the” includes plural references unless the context clearly dictates otherwise.

“Alkyl” refers to a straight or branched hydrocarbon chain radical consisting solely of carbon and hydrogen atoms, containing no unsaturation, and preferably having from one to fifteen carbon atoms (i.e., C₁-C₁₅ alkyl). In certain embodiments, an alkyl comprises one to thirteen carbon atoms (i.e., C₁-C₁₃ alkyl). In certain embodiments, an alkyl comprises one to eight carbon atoms (i.e., C₁-C₈ alkyl). In other embodiments, an alkyl comprises one to five carbon atoms (i.e., C₁-C₅ alkyl). In other embodiments, an alkyl comprises one to four carbon atoms (i.e., C₁-C₄ alkyl). In other embodiments, an alkyl comprises one to three carbon atoms (i.e., C₁-C₃ alkyl). In other embodiments, an alkyl comprises one to two carbon atoms (i.e., C₁-C₂ alkyl). In other embodiments, an alkyl comprises one carbon atom (i.e., C₁ alkyl). In other embodiments, an alkyl comprises five to fifteen carbon atoms (i.e., C₅-C₁₅ alkyl). In other embodiments, an alkyl comprises five to eight carbon atoms (i.e., C₅-C₈ alkyl). In other embodiments, an alkyl comprises two to five carbon atoms (i.e., C₂-C₅ alkyl). In other embodiments, an alkyl comprises three to five carbon atoms (i.e., C₃-C₅ alkyl). In certain embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more substituents such as those substituents described herein.

“Alkenyl” refers to a straight or branched hydrocarbon chain radical group consisting solely of carbon and hydrogen atoms, containing at least one carbon-carbon double bond, and preferably having from two to twelve carbon atoms (i.e., C₂-C₁₂ alkenyl). In certain embodiments, an alkenyl comprises two to ten carbon atoms (i.e., C₂-C₁₀ alkenyl). In certain embodiments, an alkenyl comprises two to eight carbon atoms (i.e., C₂-C₈ alkenyl). In other embodiments, an alkenyl comprises two to six carbon atoms (i.e., C₂-C₆ alkenyl). The alkenyl may be attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more substituents such as those substituents described herein.

“Alkynyl” refers to a straight or branched hydrocarbon chain radical group consisting solely of carbon and hydrogen atoms, containing at least one carbon-carbon triple bond, and preferably having from two to twelve carbon atoms (i.e., C₂-C₁₂ alkynyl). In certain embodiments, an alkynyl comprises two to eight carbon atoms (i.e., C₂-C₈ alkynyl). In other embodiments, an alkynyl comprises two to six carbon atoms (i.e., C₂-C₆ alkynyl). In other embodiments, an alkynyl comprises two to four carbon atoms (i.e., C₂-C₄ alkynyl). The alkynyl may be attached to the rest of the molecule by a single bond, for example, ethynyl, propynyl, butynyl, pentynyl, hexynyl, and the like. Unless stated otherwise specifically in the specification, an alkynyl group is optionally substituted by one or more substituents such as those substituents described herein.

“Alkylene” or “alkylene chain” refers to a straight or branched divalent hydrocarbon chain linking the rest of the molecule to a radical group, consisting solely of carbon and hydrogen, containing no unsaturation, and preferably having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group may be through any two carbons within the chain. In certain embodiments, an alkylene comprises one to ten carbon atoms (i.e., C₁-C₈ alkylene). In certain embodiments, an alkylene comprises one to eight carbon atoms (i.e., C₁-C₈ alkylene). In other embodiments, an alkylene comprises one to five carbon atoms (i.e., C₁-C₅ alkylene). In other embodiments, an alkylene comprises one to four carbon atoms (i.e., C₁-C₄ alkylene). In other embodiments, an alkylene comprises one to three carbon atoms (i.e., C₁-C₃ alkylene). In other embodiments, an alkylene comprises one to two carbon atoms (i.e., C₁-C₂ alkylene). In other embodiments, an alkylene comprises one carbon atom (i.e., C₁ alkylene). In other embodiments, an alkylene comprises five to eight carbon atoms (i.e., C₅-C₈ alkylene). In other embodiments, an alkylene comprises two to five carbon atoms (i.e., C₂-C₅ alkylene). In other embodiments, an alkylene comprises three to five carbon atoms (i.e., C₃-C₅ alkylene). Unless stated otherwise specifically in the specification, an alkylene chain is optionally substituted by one or more substituents such as those substituents described herein.

“Alkenylene” or “alkenylene chain” refers to a straight or branched divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. The points of attachment of the alkenylene chain to the rest of the molecule and to the radical group may be through any two carbons within the chain. In certain embodiments, an alkenylene comprises two to ten carbon atoms (i.e., C₂-C₁₀ alkenylene). In certain embodiments, an alkenylene comprises two to eight carbon atoms (i.e., C₂-C₈ alkenylene). In other embodiments, an alkenylene comprises two to five carbon atoms (i.e., C₂-C₅ alkenylene). In other embodiments, an alkenylene comprises two to four carbon atoms (i.e., C₂-C₄ alkenylene). In other embodiments, an alkenylene comprises two to three carbon atoms (i.e., C₂-C₃ alkenylene). In other embodiments, an alkenylene comprises two carbon atom (i.e., C₂ alkenylene). In other embodiments, an alkenylene comprises five to eight carbon atoms (i.e., C₅-C₈ alkenylene). In other embodiments, an alkenylene comprises three to five carbon atoms (i.e., C₃-C₅ alkenylene). Unless stated otherwise specifically in the specification, an alkenylene chain is optionally substituted by one or more substituents such as those substituents described herein.

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. The points of attachment of the alkynylene chain to the rest of the molecule and to the radical group may be through any two carbons within the chain. In certain embodiments, an alkynylene comprises two to ten carbon atoms (i.e., C₂-C₁₀ alkynylene). In certain embodiments, an alkynylene comprises two to eight carbon atoms (i.e., C₂-C₈ alkynylene). In other embodiments, an alkynylene comprises two to five carbon atoms (i.e., C₂-C₅ alkynylene). In other embodiments, an alkynylene comprises two to four carbon atoms (i.e., C₂-C₄ alkynylene). In other embodiments, an alkynylene comprises two to three carbon atoms (i.e., C₂-C₃ alkynylene). In other embodiments, an alkynylene comprises two carbon atom (i.e., C₂ alkynylene). In other embodiments, an alkynylene comprises five to eight carbon atoms (i.e., C₅-C₈ alkynylene). In other embodiments, an alkynylene comprises three to five carbon atoms (i.e., C₃-C₅ alkynylene). Unless stated otherwise specifically in the specification, an alkynylene chain is optionally substituted by one or more substituents such as those substituents described herein.

“Aryl” refers to an aromatic monocyclic or aromatic multicyclic hydrocarbon ring system. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents such as those substituents described herein.

“Aralkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as described above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as described above for an alkynylene chain.

The term “C_{x-y}” or “Cₓ-C_{y}” when used in conjunction with a chemical moiety, such as alkyl, alkenyl, or alkynyl is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain. The terms “C_{x-y}alkenyl” and “C_{x-y}alkynyl” refer to substituted or unsubstituted unsaturated aliphatic groups analogous in length and possible substitution to the alkyls described above, but that contain at least one double or triple bond respectively.

“Carbocycle” refers to a saturated, unsaturated or aromatic rings in which each atom of the ring is carbon. Carbocycle may be monocyclic or polycyclic and may include 3- to 10-membered monocyclic rings, 6- to 12-membered bicyclic rings, and 6- to 12-membered bridged rings. Each ring of a bicyclic carbocycle may be selected from saturated, unsaturated, and aromatic rings. In some embodiments, the carbocycle is an aryl. In some embodiments, the carbocycle is a cycloalkyl. In some embodiments, the carbocycle is a cycloalkenyl. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl. Unless stated otherwise specifically in the specification, a carbocycle is optionally substituted by one or more substituents such as those substituents described herein.

“Cycloalkyl” refers to a saturated ring in which each atom of the ring is carbon. Cycloalkyl may include monocyclic and polycyclic rings such as 3- to 10-membered monocyclic rings, 6- to 12-membered bicyclic rings, and 6- to 12-membered bridged rings. In certain embodiments, a cycloalkyl comprises three to ten carbon atoms. In other embodiments, a cycloalkyl comprises five to seven carbon atoms. The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl. Polycyclic cycloalkyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “cycloalkyl” is meant to include cycloalkyl radicals that are optionally substituted by one or more substituents such as those substituents described herein.

“Cycloalkenyl” refers to a saturated ring in which each atom of the ring is carbon and there is at least one double bond between two ring carbons. Cycloalkenyl may include monocyclic and polycyclic rings such as 3- to 10-membered monocyclic rings, 6- to 12-membered bicyclic rings, and 6- to 12-membered bridged rings. In other embodiments, a cycloalkenyl comprises five to seven carbon atoms. The cycloalkenyl may be attached to the rest of the molecule by a single bond. Examples of monocyclic cycloalkenyls include, e.g., cyclopentenyl, cyclohexenyl, cycloheptenyl, and cyclooctenyl. Unless otherwise stated specifically in the specification, the term “cycloalkenyl” is meant to include cycloalkenyl radicals that are optionally substituted by one or more substituents such as those substituents described herein.

“Haloalkyl” refers to an alkyl radical, as defined above, that is substituted by one or more halo radicals, for example, trifluoromethyl, dichloromethyl, bromomethyl, 2,2,2-trifluoroethyl, 1-chloromethyl-2-fluoroethyl, and the like. In some embodiments, the alkyl part of the haloalkyl radical is optionally substituted as described herein.

“Heterocycle” refers to a saturated, unsaturated or aromatic ring comprising carbon atoms and one or more heteroatoms. Exemplary heteroatoms include N, O, Si, P, B, and S atoms. Heterocycle may be monocyclic or polycyclic and may include 3- to 10-membered monocyclic rings, 6- to 12-membered bicyclic rings, and 6- to 12-membered bridged rings. Each ring of a bicyclic heterocycle may be selected from saturated, unsaturated, and aromatic rings. In some embodiments, the heterocycle is a heteroaryl. In some embodiments, the heterocycle is a heterocycloalkyl. In an exemplary embodiment, a heterocycle, e.g., pyridyl, may be fused to a saturated or unsaturated ring, e.g., cyclohexane, cyclopentane, or cyclohexene.

“Heterocycloalkyl” refers to a saturated ring with carbon atoms and at least one heteroatom. Exemplary heteroatoms include N, O, Si, P, B, and S atoms. Heterocycloalkyl may include monocyclic and polycyclic rings such as 3- to 10-membered monocyclic rings, 6- to 12-membered bicyclic rings, and 6- to 12-membered bridged rings. The heteroatoms in the heterocycloalkyl radical are optionally oxidized. One or more nitrogen atoms, if present, are optionally quaternized. The heterocycloalkyl is attached to the rest of the molecule through any atom of the heterocycloalkyl, valence permitting, such as any carbon or nitrogen atoms of the heterocycloalkyl. Examples of heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocycloalkyl” is meant to include heterocycloalkyl radicals as defined above that are optionally substituted by one or more substituents such as those substituents described herein.

“Heteroaryl” refers to an aromatic ring comprising carbon atoms and one or more heteroatoms. Exemplary heteroatoms include N, O, Si, P, B, and S atoms. As used herein, the heteroaryl ring may be selected from monocyclic or bicyclic and fused or bridged ring systems rings wherein at least one of the rings in the ring system is aromatic, i.e., it contains a cyclic, delocalized (4n+2) π-electron system in accordance with the Hückel theory. The heteroatom(s) in the heteroaryl radical may be optionally oxidized. One or more nitrogen atoms, if present, are optionally quaternized. The heteroaryl may be attached to the rest of the molecule through any atom of the heteroaryl, valence permitting, such as a carbon or nitrogen atom of the heteroaryl. Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, the term “heteroaryl” is meant to include heteroaryl radicals as defined above which are optionally substituted by one or more substituents such as those substituents described herein.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a phenyl ring.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. Unless otherwise stated, chemical structures depicted herein are intended to include structures which are different tautomers of the structures depicted. For example, the chemical structure depicted with an enol moiety also includes the keto tautomer form of the enol moiety. The exact ratio of the tautomers depends on several factors, including physical state, temperature, solvent, and pH. Some examples of tautomeric equilibrium include:

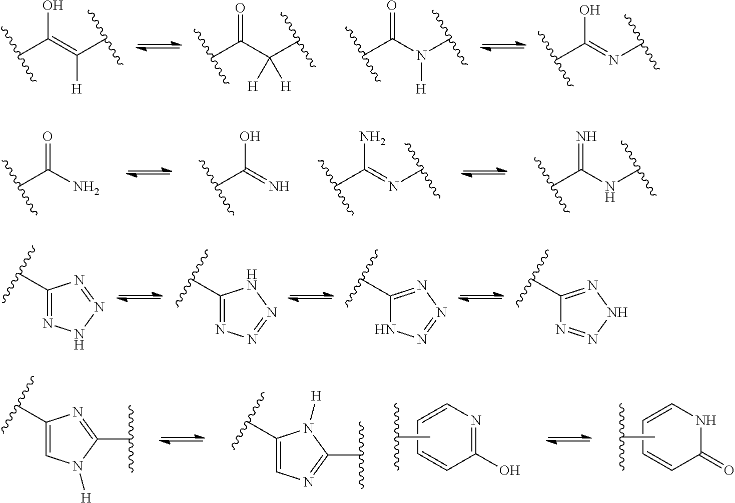

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as deuterium (²H), tritium (³H), iodine-125 (¹²⁵I) or carbon-14 (¹⁴C). Isotopic substitution with ²H, C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commerically from chemical vendors, such as Aldrich Chemical Co.

Deuterium-transfer reagents suitable for use in nucleophilic substitution reactions, such as iodomethane-d₃ (CD₃I), are readily available and may be employed to transfer a deuterium-substituted carbon atom under nucleophilic substitution reaction conditions to the reaction substrate. The use of CD₃I is illustrated, by way of example only, in the reaction schemes below.

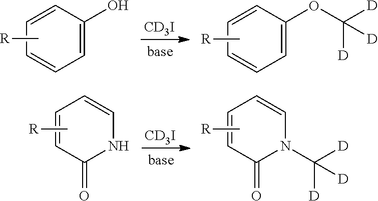

Deuterium-transfer reagents, such as lithium aluminum deuteride (LiAlD₄), are employed to transfer deuterium under reducing conditions to the reaction substrate. The use of LiAlD₄ is illustrated, by way of example only, in the reaction schemes below.

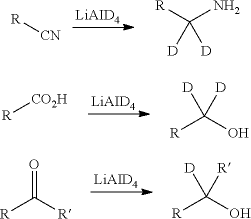

Deuterium gas and palladium catalyst are employed to reduce unsaturated carbon-carbon linkages and to perform a reductive substitution of aryl carbon-halogen bonds as illustrated, by way of example only, in the reaction schemes below.

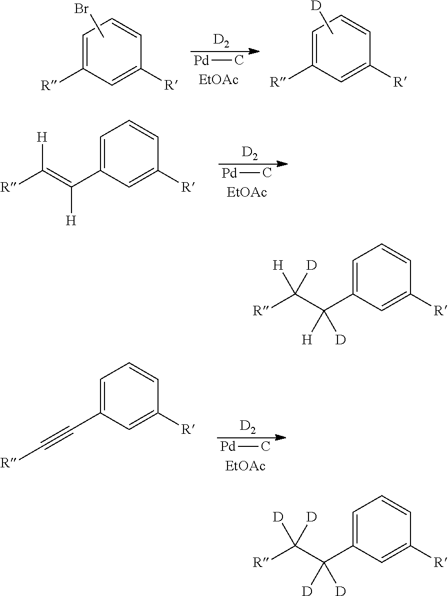

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier is “acceptable” in the sense of being compatible with the other ingredients of the formulation and not injurious to the patient. Some examples of materials which can serve as pharmaceutically acceptable carriers include: (1) sugars, such as lactose, glucose and sucrose: (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In certain embodiments, the term “prevent” or “preventing” as related to a disease or disorder may refer to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “substituted” refers to moieties having substituents replacing a hydrogen on one or more carbons or heteroatoms of the structure. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms.

Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, a carbocycle, a heterocycle, a cycloalkyl, a heterocycloalkyl, an aromatic and heteroaromatic moiety. In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂(where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, hydroxy, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂(where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

The terms “treat,” “treating” or “treatment,” as used herein, may include alleviating, abating or ameliorating a disease or condition symptoms, preventing additional symptoms, ameliorating or preventing the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

Compounds of the present invention also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

### Introduction

Alpha-crystallin is a major structural protein found in the eye and can maintain the refractive index and transparency of the lens. Alpha-crystallin is composed of two homologous subunits: alphaA-crystallin (cryAA) and alphaB-crystallin (cryAB), which belong to a family of small heat shock proteins (sHSPs) that contain a conserved crystallin domain. AlphaA is 173 amino acids long and alphaB is 175 amino acid long. The two alpha-crystallin genes, alphaA and alphaB, encode for proteins that share 57% sequence identity. The ratio of alphaA to alphaB in most vertebrate lenses can be 3:1 but this ratio can vary with species and age. The alphaA-crystallin protein can be found mostly in the lens and only in few other tissues whereas alphaB-crystallin protein can be ubiquitously expressed and can be found in other tissues, such as brain, heart and muscle.

These alpha-crystallin subunits act as molecular chaperones to prevent the cellular aggregation and inactivation of client proteins under a variety of stress conditions. However, the chaperone activity of these alpha-crystallin subunits can be lost or deteriorated during aging or due to certain genetic or environment factors, which can cause aggregation and precipitation of alpha-crystallin and lead to cataracts.

In certain embodiments, the disclosure provides compounds, formulations and methods for treating vision disorders associated with alpha-crystallin protein aggregation in the lens. In particular, the disclosure provides compounds, formulations and methods for treating cataract and presbyopia.

### Compounds of the Disclosure

The present disclosure provides compounds and salts, and formulations thereof, for use in the treatment of ophthalmic diseases. The disclosed compounds and salts can be used, for example, for the treatment or prevention of vision disorders such as near vision impairment. In certain embodiments, the compounds of the disclosure reduce alpha-crystallin protein aggregation in the lens of an eye. Compounds and salts of the disclosure may be used in the formulations, methods and combination therapies described herein. In certain embodiments, compounds and salts of the disclosure are used in the treatment or prevention of cataract or presbyopia.

In some embodiments, the compound is of the formula III:

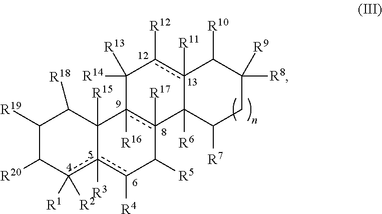

or a salt thereof, wherein:

R¹, R², R³, R⁴, R⁶, R⁸, R⁹, R¹¹, R¹², R¹³, R¹⁴, R¹⁵, R¹⁶, and R¹⁷ are independently selected from hydrogen, halogen, —OR³⁰, —SR³⁰, —OSO₃R³⁰, —OPO₃R³⁰, —N(R³¹)₂, —C(O)R³⁰, —C(O)OR³⁰, —OC(O)R³⁰, —NO₂, —CN, optionally substituted C₁-C₁₀ alkyl, optionally substituted C₂-C₁₀ alkenyl, optionally substituted C₂-C₁₀ alkynyl, optionally substituted carbocycle and optionally substituted heterocycle; R¹ taken together with R² is further selected from ═O, ═S, and ═N(R³¹); R⁸ taken together with R⁹ is further selected from ═O, ═S, and ═N(R³¹); R¹³ taken together with R¹⁴ is further selected from ═O, ═S, and ═N(R³¹); R⁹ and R¹⁰ taken together with the atoms to which they are attached may further form an optionally substituted carbocycle or optionally substituted heterocycle; and wherein R³ is absent when there is a double bond between carbons 5 and 6, R¹⁶ and R¹⁷ are absent when there is a double bond between carbons 8 and 9, R¹¹ is absent when there is a double bond between carbons 12 and 13; and R² and R³ are absent and there is a single bond between carbons 5 and 6 when there is a double bond between carbons 4 and 5;

R⁵, R⁷, R¹⁰, R¹⁸, R¹⁹ and R²⁰ are independently selected from hydrogen, halogen, —OR³⁰, —SR³⁰, —OSO₃R³⁰, —OPO₃R³⁰, N(R³¹)₂, —C(O)R³⁰, —C(O)OR³⁰, —OC(O)R³⁰, —NO₂, —CN, ═O, ═S, ═N(R³¹), optionally substituted C₁-C₁₀ alkyl, optionally substituted C₂-C₁₀ alkenyl, C₂-C₁₀ alkynyl, optionally substituted carbocycle and optionally substituted heterocycle;

each R³¹ is independently selected from hydrogen, —OR³⁰, —SR³⁰, —S(O)R³⁰, —S(O)₂R³⁰, —C(O)R³⁰, —C(O)OR³⁰, optionally substituted C₁-C₁₀ alkyl, optionally substituted C₂-C₁₀ alkenyl, C₂-C₁₀ alkynyl, optionally substituted carbocycle and optionally substituted heterocycle;

each R³⁰ is independently selected from hydrogen, optionally substituted C₁-C₆ alkyl, optionally substituted C₂-C₆ alkenyl, optionally substituted C₂-C₆ alkynyl, optionally substituted carbocycle and optionally substituted heterocycle; and

n is selected from 0 or 1.

For a compound or salt of Formula (III), a dotted line in the structure depicts an optional double bond at this position. In certain embodiments, the compound or salt of Formula (III) has a double bond between carbons 5 and 6 or carbons 8 and 9. For a compound or salt of Formula (III), if there is a double bond between carbons 4 and 5, then there is a single bond between carbons 5 and 6. In certain embodiments, substituents on a doubly-bound carbon atom may tautomerize and the tautomers are included within the scope of the disclosure. For example, when there is a double bond between carbons 5 and 6 and R⁴ is hydroxyl, the keto tautomer, i.e., R⁴ is oxo (═O) and there is a single bond between carbons 5 and 6, is included within the scope of the disclosure.

When referring to structures herein, particularly with respect to references to optional double bonds in Formula (III), a carbon number may be used. The carbon numbers may appear next to the carbon atoms to which the text refers. For example, “carbon 4” refers to the atom bearing substituents R¹ and R² as depicted in Figure (III).

When R¹, R², R³, R⁴, R⁵, R⁶, R⁷ R⁸, R⁹, R¹⁰, R¹¹, R¹², R¹³, R¹⁴, R¹⁵, R¹⁶, R¹⁷, R¹⁸, R¹⁹, R²⁰, R³⁰ and R³¹ is described as optionally substituted, the substituents may be independently selected at each occurrence from halogen, —NO₂, —CN, —OR⁴⁰, —SR⁴⁰, —N(R⁴⁰)₂, —S(═O)R⁴⁰, —S(═O)₂R⁴⁰, —S(═O)₂N(R⁴⁰)₂, —NR⁴⁰S(═O)₂R⁴⁰, —C(O)R⁴⁰, —C(O)OR⁴⁰, —OC(O)R⁴⁰, —OC(O)OR⁴⁰, —OC(O)N(R⁴⁰)₂, —NR⁴⁰C(O)R⁴⁰, —C(O)N(R⁴⁰)₂, ═O, ═S, ═N(R⁴⁰), —P(O)(OR⁴⁰)₂, —OP(O)(OR⁴⁰)₂; C₁₋₁₀ alkyl, C₂₋₁₀ alkenyl, and C₂₋₁₀ alkynyl, each of which is independently optionally substituted at each occurrence with one or more substituents selected from halogen, —NO₂, —CN, —OR⁴⁰, —SR⁴⁰, —N(R⁴⁰)₂, —S(═O)R⁴⁰, —S(═O)₂R⁴⁰, —S(═O)₂N(R⁴⁰)₂, —NR⁴⁰S(═O)₂R⁴⁰, —C(O)R⁴⁰, —C(O)OR⁴⁰, —OC(O)R⁴⁰, —OC(O)OR⁴⁰, —OC(O)N(R⁴⁰)₂, —NR⁴⁰C(O)R⁴⁰, —C(O)N(R⁴⁰)₂, ═O, ═S, ═N(R⁴⁰), —P(O)(OR⁴⁰)₂, —OP(O)(OR⁴⁰)₂, C₃₋₁₂ carbocycle and 3- to 12-membered heterocycle; and C₃₋₁₂ carbocycle and 3- to 12-membered heterocycle, wherein each C₃₋₁₂ carbocycle and 3- to 12-membered heterocycle is independently optionally substituted with one or more substituents selected from halogen, —NO₂, —CN, —OR⁴⁰, —SR⁴⁰, —N(R⁴⁰)₂, —S(═O)R⁴⁰, —S(═O)₂R⁴⁰, —S(═O)₂N(R⁴⁰)₂, —NR⁴⁰S(═O)₂R⁴⁰, —C(O)R⁴⁰, —C(O)OR⁴⁰, —OC(O)R⁴⁰, —OC(O)OR⁴⁰, —OC(O)N(R⁴⁰)₂, —NR⁴⁰C(O)R⁴⁰, —C(O)N(R⁴⁰)₂, ═O, ═S, ═N(R⁴⁰), —P(O)(OR⁴⁰)₂, —OP(O)(OR⁴⁰)₂, C₁₋₆ alkyl, C₁₋₆ haloalkyl, C₂₋₆ alkenyl, and C₂₋₆ alkynyl and wherein R⁴⁰ at each occurrence is independently selected from hydrogen; and C₁₋₆ alkyl, C₂₋₆ alkenyl, C₂₋₆ alkynyl, C₃₋₁₂ carbocycle and 3- to 12-membered heterocycle, each of which may be optionally substituted by halogen, —CN, —NO₂, —OH, ═O and —OCH₃.

When R¹, R², R³, R⁴, R⁵, R⁶, R⁷ R⁸, R⁹, R¹⁰, R¹¹, R¹², R¹³, R¹⁴, R¹⁵, R¹⁶, R¹⁷, R¹⁸, R¹⁹, R²⁰, R³⁰ and R³¹ is described as optionally substituted, the substituents may be independently selected at each occurrence from halogen, —NO₂, —CN, —OR⁴⁰, —SR⁴⁰, —N(R⁴⁰)₂, —S(═O)R⁴⁰, —S(═O)₂R⁴⁰, —S(═O)₂N(R⁴⁰)₂, —NR⁴⁰S(═O)₂R⁴⁰, —C(O)R⁴⁰, —C(O)OR⁴⁰, —OC(O)R⁴⁰, —OC(O)OR⁴⁰, —OC(O)N(R⁴⁰)₂, —NR⁴⁰C(O)R⁴⁰, —C(O)N(R⁴⁰)₂, ═O, ═S, ═N(R⁴⁰), —P(O)(OR⁴⁰)₂, —OP(O)(OR⁴⁰)₂; C₁₋₁₀ alkyl, C₂₋₁₀ alkenyl, and C₂₋₁₀ alkynyl, each of which is independently optionally substituted at each occurrence with one or more substituents selected from halogen, —NO₂, ═O, —CN, and —OR⁴⁰ and wherein R⁴⁰ at each occurrence is independently selected from hydrogen; and C₁₋₆ alkyl, C₂₋₆ alkenyl, C₂₋₆ alkynyl, C₃₋₁₂ carbocycle and 3- to 12-membered heterocycle, each of which may be optionally substituted by halogen, —CN, —NO₂, —OH, ═O and —OCH₃.

In certain embodiments, n is 0 for a compound or salt of Formula (III). In certain embodiments, n is 1 for a compound or salt of Formula (III).

In certain embodiments, a compound of Formula (III) is represented by any of the following Formulas (IVA), (IVB), (IVC), (IVD), (IVE), (IVF), (IVG) and (IVH):

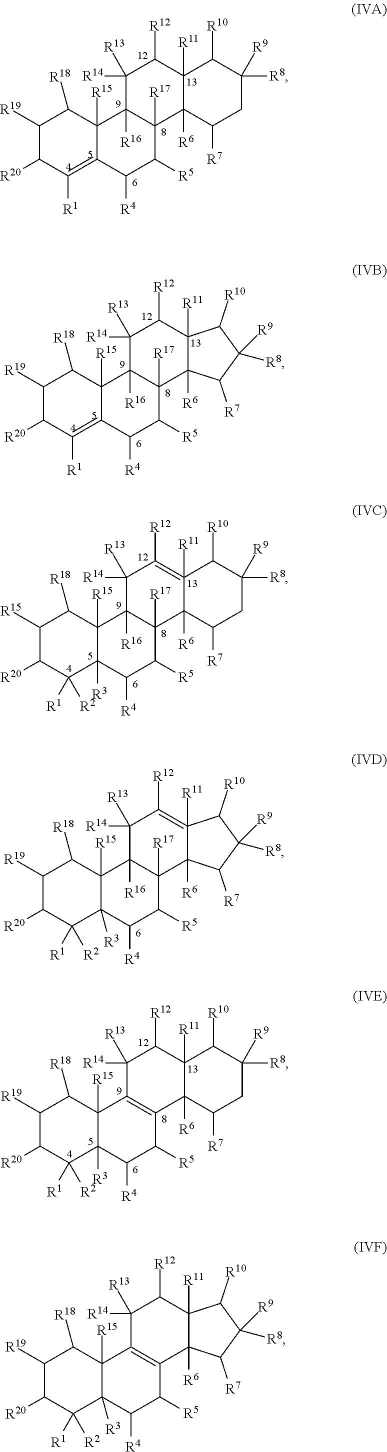

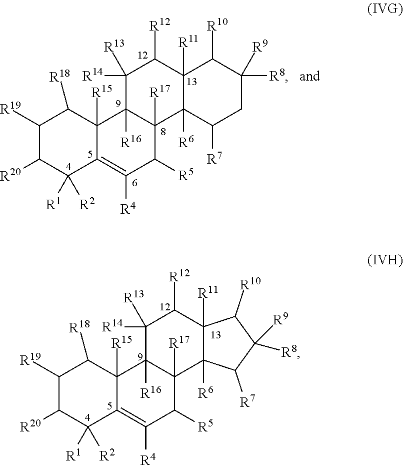

or a salt of any one thereof.

For a compound or salt of Formula (III), (IVA), (IVB), (IVC), (IVD), (IVE), (IVF), (IVG) and (IVH), R¹, R², R³, R⁴, R⁶, R⁸, R⁹, R¹¹, R¹², R¹³, R¹⁴, R¹⁵, R¹⁶, and R¹⁷ may be independently selected from hydrogen, halogen, —OR³⁰, —SR³⁰, —OSO₃R³⁰, —OPO₃R³⁰, N(R³¹)₂, —C(O)R³⁰, —C(O)OR³⁰, —OC(O)R³⁰, —NO₂, —CN, and optionally substituted C₁-C₁₀ alkyl. R¹, R², R³, R⁴, R⁶, R⁸, R⁹, R¹¹, R¹², R¹³, R¹⁴, R¹⁵, R¹⁶, and R¹⁷ may be independently selected from hydrogen, halogen, —OR³⁰, —NO₂, —CN, and optionally substituted C₁-C₁₀ alkyl. For a compound or salt of Formula (III), R⁵, R⁷, R¹⁰, R¹⁸, R¹⁹ and R²⁰ may be independently selected from hydrogen, halogen, —OR³⁰, —SR³⁰, —OSO₃R³⁰, —OPO₃R³⁰, —N(R³¹)₂, —C(O)R³⁰, —C(O)OR³⁰, —OC(O)R³⁰, —NO₂, —CN, ═O, ═S, ═N(R³¹), optionally substituted C₁-C₁₀ alkyl, optionally substituted C₂-C₁₀ alkenyl, and optionally substituted C₂-C₁₀ alkynyl.

For a compound or salt of Formula (III), (IVA), (IVB), (IVC), (IVD), (IVE), (IVF), (IVG) and (IVH), R¹ and R² may be independently selected from hydrogen, halogen, —OR³⁰ and optionally substituted C₁-C₁₀ alkyl, or R¹ taken together with R² is selected from ═O, ═S, and ═N(R³¹). In certain embodiments, R¹ and R² are independently selected from hydrogen, fluoro, chloro, bromo, iodo, methyl, or ethyl. In certain embodiments, R¹ and R² are each hydrogen. In certain embodiments, R¹ and R² are each methyl.

For a compound or salt of Formula (III), (IVA), (IVB), (IVC), (IVD), (IVE), (IVF), (IVG) and (IVH), R³ may be selected from hydrogen, —OR³⁰ and optionally substituted C₁-C₁₀ alkyl and there is a single bond between carbons 4 and 5 and a single bond between carbons 5 and 6. In certain embodiments, R³ is selected from hydrogen and methyl. In particular embodiments, R³ is hydrogen and there is a single bond between carbons 4 and 5 and a single bond between carbons 5 and 6.

For a compound or salt of Formula (III), (IVA), (IVB), (IVC), (IVD), (IVE), (IVF), (IVG) and (IVH), R³ is absent when there is a double bond between carbons 5 and 6. For a compound or salt of Formula (III), R² and R³ are absent when there is a double bond between carbons 4 and 5.

For a compound or salt of Formula (III), (IVA), (IVB), (IVC), (IVD), (IVE), (IVF), (IVG) and (IVH), R²⁰ may be selected from hydrogen, halogen, —OR³⁰, —SR³⁰, —OSO₃R³⁰, —OPO₃R³⁰, —N(R³¹)₂, —C(O)R³⁰, —C(O)OR³⁰, —OC(O)R³⁰, —NO₂, —CN, ═O, ═S, ═N(R³¹), optionally substituted C₁-C₁₀ alkyl. R²⁰ may be selected from hydrogen, halogen, —OR³⁰, —SR³⁰, —OSO₃R³⁰, —OPO₃R³⁰, —N(R³¹)₂, —C(O)R³⁰, —C(O)OR³⁰, —OC(O)R³⁰, —NO₂, —CN, ═O, ═S, and ═N(R³¹). In certain embodiments, R²⁰ is selected from —OR³⁰, —OSO₃R³⁰, —OPO₃R³⁰, —C(O)R³⁰, —C(O)OR³⁰, —OC(O)R³⁰, ═O, and ═S. In preferred embodiments, R²⁰ is selected from —OR³⁰, such as hydroxyl, and ═O.

For a compound or salt of Formula (III), (IVA), (IVB), (IVC), (IVD), (IVE), (IVF), (IVG) and (IVH), R⁴ may be selected from hydrogen, halogen, —OR³⁰ and optionally substituted C₁-C₁₀ alkyl. R⁴ may be selected from hydrogen and —OR³⁰, such as hydroxyl. In preferred embodiments, R⁴ is hydrogen.

For a compound or salt of Formula (III), (IVA), (IVB), (IVC), (IVD), (IVE), (IVF), (IVG) and (IVH), R⁵ may be selected from hydrogen, halogen, ═O, —OR³⁰ and optionally substituted C₁-C₁₀ alkyl. In certain embodiments, R⁵ is selected from hydrogen, halogen and C₁-C₁₀ alkyl. In preferred embodiments, R⁵ is hydrogen.

For a compound or salt of Formula (III), (IVA), (IVB), (IVC), (IVD), (IVE), (IVF), (IVG) and (IVH), R⁶ may be selected from hydrogen, halogen, —OR³⁰ and optionally substituted C₁-C₁₀ alkyl. R⁶ may be selected from hydrogen, halogen and C₁-C₁₀ alkyl. In certain embodiments, R⁶ is hydrogen. In certain embodiments, R⁶ is methyl.

For a compound or salt of Formula (III), (IVA), (IVB), (IVC), (IVD), (IVE), (IVF), (IVG) and (IVH), R⁷ may be selected from hydrogen, halogen, —OR³⁰, ═O, and optionally substituted C₁-C₁₀ alkyl. R⁷ may be selected from hydrogen, halogen and C₁-C₁₀ alkyl. In certain embodiments, R⁷ is hydrogen.

For a compound or salt of Formula (III), (IVA), (IVB), (IVC), (IVD), (IVE), (IVF), (IVG) and (IVH), R⁸ and R⁹ may be independently selected from hydrogen, halogen, —OR³⁰ and optionally substituted C₁-C₁₀ alkyl. R⁸ and R⁹ may be independently selected from hydrogen, halogen, and C₁-C₁₀ alkyl.

For a compound or salt of Formula (III), (IVA), (IVB), (IVC), (IVD), (IVE), (IVF), (IVG) and (IVH), R⁹ and R¹⁰ taken together with the atoms to which they are attached may form an optionally substituted carbocycle or optionally substituted heterocycle. R⁹ and R¹⁰ taken together with the atoms to which they are attached may form an optionally substituted carbocycle, such as an optionally substituted 5- or 6-membered carbocycle.

For a compound or salt of Formula (III), (IVA), (IVB), (IVC), (IVD), (IVE), (IVF), (IVG) and (IVH), R¹⁰ may be selected from —OR³⁰, ═O, ═S, ═N(R³¹), optionally substituted C₁-C₁₀ alkyl, optionally substituted C₂-C₁₀ alkenyl, and optionally substituted C₂-C₁₀ alkynyl. R¹⁰ may be selected from —OR³⁰ or ═O. R¹⁰ may be optionally substituted C₁-C₁₀ alkyl or optionally substituted C₂-C₁₀ alkenyl. In certain embodiments, R¹⁰ is substituted with one or more substituents independently selected from: halogen, —OR³⁰, —SR³⁰, —OSO₃R³⁰, —OPO₃R³⁰, —N(R³¹)₂, —C(O)R³⁰, —C(O)OR³⁰, —OC(O)R³⁰, —NO₂, —CN, ═O, ═S, and ═N(R³¹). R¹⁰ may be substituted with one or more substituents independently selected from: halogen, —OR³⁰, —SR³⁰, —N(R³¹)₂, —C(O)R³⁰, —C(O)OR³⁰, —OC(O)R³⁰, —NO₂, —CN, ═O, ═S, and ═N(R³¹). In certain embodiments, R¹⁰ is C₁-C₁₀ alkyl substituted with one or more substituents selected from halogen and —OR³⁰. In certain embodiments, R¹⁰ is C₈ alkyl substituted with one or more substituents selected from halogen and —OR³⁰. In certain embodiments, R¹⁰ is C₈ alkyl substituted with —OR³⁰, e.g., hydroxyl. In certain embodiments, R¹⁰ is C₂-C₁₀ alkenyl optionally substituted with one or more substituents selected from halogen and —OR³⁰ e.g., hydroxyl.

For a compound or salt of Formula (III), (IVA), (IVB), (IVC), (IVD), (IVE), (IVF), (IVG) and (IVH), R¹¹ may be selected from hydrogen, —OR³⁰ and optionally substituted C₁-C₁₀ alkyl. In certain embodiments, R¹¹ is hydrogen or C₁-C₁₀ alkyl. In certain embodiments, R¹¹ is methyl. In certain embodiments, R¹¹ is absent and there is a double bond between carbons 12 and 13.

For a compound or salt of Formula (III), (IVA), (IVB), (IVC), (IVD), (IVE), (IVF), (IVG) and (IVH), R¹² may be selected from hydrogen, halogen, —OR³⁰ and optionally substituted C₁-C₁₀ alkyl. R¹² may be selected from hydrogen and —OR³⁰. In certain embodiments, R¹² is hydrogen.

For a compound or salt of Formula (III), (IVA), (IVB), (IVC), (IVD), (IVE), (IVF), (IVG) and (IVH), R¹³ and R¹⁴ may be independently selected from hydrogen, halogen, —OR³⁰ and optionally substituted C₁-C₁₀ alkyl, or R¹³ taken together with R¹⁴ is selected from ═O, ═S, and ═N(R³¹). In certain embodiments, R¹³ and R¹⁴ are independently selected from hydrogen, halogen, —OR³⁰, and optionally substituted C₁-C₁₀ alkyl. In certain embodiments, R¹³ and R¹⁴ are each hydrogen. In certain embodiments, R¹³ taken together with R¹⁴ is ═O.

For a compound or salt of Formula (III), (IVA), (IVB), (IVC), (IVD), (IVE), (IVF), (IVG) and (IVH), R¹⁵ is selected from hydrogen and optionally substituted C₁-C₁₀ alkyl. In certain embodiments, R¹⁵ is methyl.

For a compound or salt of Formula (III), (IVA), (IVB), (IVC), (IVD), (IVE), (IVF), (IVG) and (IVH), R¹⁶ and R¹⁷ may each be hydrogen. In certain embodiments, R¹⁶ and R¹⁷ are both absent and there is a double bond between carbons 8 and 9.

For a compound or salt of Formula (III), (IVA), (IVB), (IVC), (IVD), (IVE), (IVF), (IVG) and (IVH), R¹⁸ and R¹⁹ may be independently selected from hydrogen, halogen, —OR³⁰, ═O, and optionally substituted C₁-C₁₀ alkyl. In certain embodiments, R¹⁸ and R¹⁹ are each hydrogen.

For a compound or salt of Formula (III), the compound of Formula (III) may be represented by Formula (IIIA):

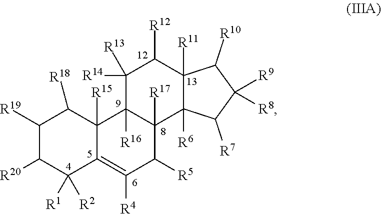

or a salt thereof.

For a compound or salt of Formula (III), the compound of Formula (IIIA) may be represented by the Formula (IIIB).

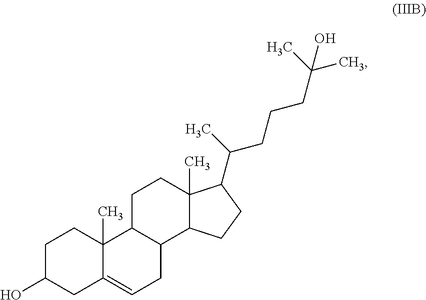 [SMILES: CC(CCCC(C)(C)O)C1CCC2C3CC=C4CC(O)CCC4(C)C3CCC12C]

or a salt thereof.

For a compound or salt of Formula (III), the compound of Formula (IIIA) may be represented by the Formula (IIIC):

or a salt thereof.

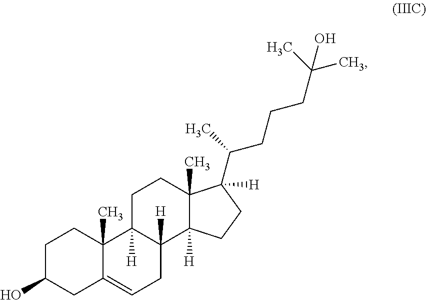

For a compound or salt of Formula (III), the compound of Formula (III) may be represented by Formula (IIID):

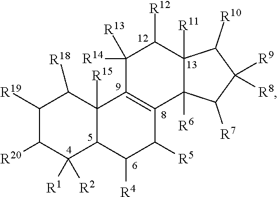

or a salt thereof. In certain embodiments, the compound or salt of Formula (III) is lanosterol or a salt thereof. In certain embodiments, the compound or salt of Formula (III) is not lanosterol.

In certain embodiments, the compound or salt of Formula (III) is not cholesterol. In certain embodiments, the compound or salt of Formula (III) is not 25-hydroxycholesterol. In certain embodiments, the compound of Formula (III) is not lanosterol.

The disclosure provides a compound of formula I:

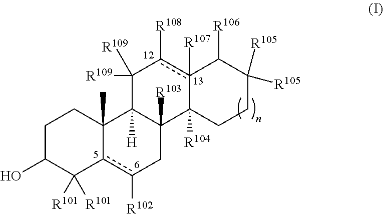

wherein:

each R¹⁰¹ are H or each R¹⁰¹ are Me;

R¹⁰² is H or OH;

the dashed line between carbons 5 and 6 indicates an optional double bond;

R¹⁰³ is H or Me;

R¹⁰⁴ is H or Me;

n is 0 or 1;

(a) R¹⁰⁶ is

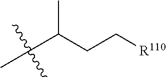

and each R¹⁰⁵ is independently H or Me or (b) R¹⁰⁶ and one R¹⁰⁵ taken together form an optionally substituted 6-membered ring and the other R¹⁰⁵ is Me;

the dashed line between carbons 12 and 13 is an optional double bond, with the proviso that R¹⁰⁷ is not present when the double bond between carbons 12 and 13 is present, and R¹⁰⁷ is H or Me when the double bond between carbons 12 and 13 is not present;

R¹⁰⁸ is H or OH;

both R¹⁰⁹ together form an oxo (═O) or both R¹⁰⁹ are hydrogen; and

R¹¹⁰ is CO₂H or linear or branched C₁-C₆ alkyl;

or a prodrug or pharmaceutically acceptable salt thereof.

In some embodiments, the compound of formula I has a structure of formula IA or formula IB:

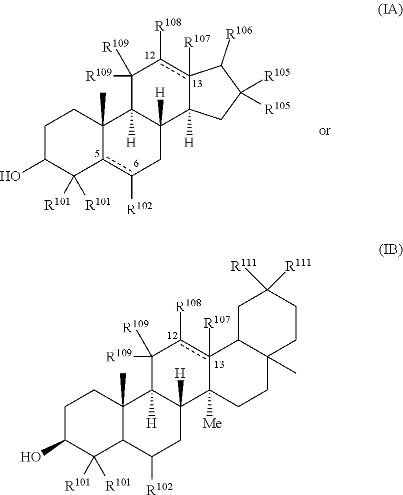

wherein each R¹¹¹ is independently alkyl, CO₂H, or CO₂ alkyl.

In some embodiments, the compound has a structure of formula II:

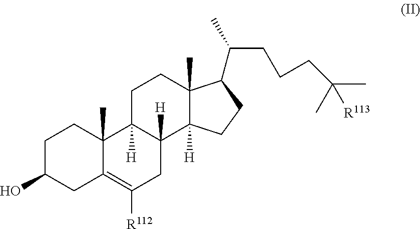

wherein R¹¹² is H or OH and R¹¹³ is H or OH.

In some embodiments, the compound is 5-cholesten-3b,25-diol.

In some embodiments, the compound is 5a-cholestan-3b-ol-6-one.

In some embodiments, the compound is of the formula:

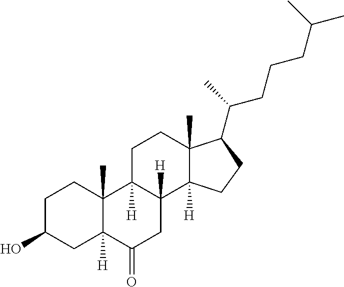

In some embodiments, the compound is of the formula:

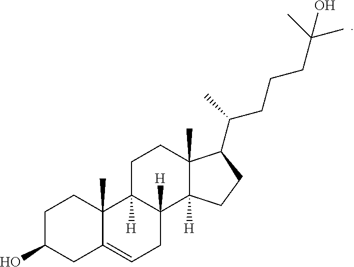

In certain embodiments, the compound of Formula (I) or (II) is not cholesterol. In certain embodiments, the compound of Formula (I) or (II) is not lithocholic acid.

The invention provides salts of any one of Formulas (I), (IA), (IB), (II), (III), (IIIA), (IIIB), (IIIC), (IIID), (IVA), (IVB), (IVC), (IVD), (IVE), (IVF), (IVG) and (IVH). Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt.

Metal salts can arise from the addition of an inorganic base to a compound of the invention. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the invention. In some embodiments, the organic amine is triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrrazole, pipyrrazole, imidazole, pyrazine, or pipyrazine.

In some embodiments, an ammonium salt is a triethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrrazole salt, an imidazole salt, or a pyrazine salt.

Acid addition salts can arise from the addition of an acid to a compound of the invention. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisinic acid, gluconic acid, glucaronic acid, saccaric acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, or maleic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisinate salt, a gluconate salt, a glucaronate salt, a saccarate salt, a formate salt, a benzoate salt, a glutamate salt, a pantothenate salt, an acetate salt, a propionate salt, a butyrate salt, a fumarate salt, a succinate salt, a methanesulfonate (mesylate) salt, an ethanesulfonate salt, a benzenesulfonate salt, a p-toluenesulfonate salt, a citrate salt, an oxalate salt, or a maleate salt.

The compounds of any one of Formulas (I), (IA), (IB), (II), (III), (IIIA), (IIIB), (IIIC), (IIID), (IVA), (IVB), (IVC), (IVD), (IVE), (IVF), (IVG) and (IVH) may in some cases exist as diastereomers, enantiomers, or other stereoisomeric forms. The compounds and salts presented herein include all diastereomeric, enantiomeric, and epimeric forms as well as the appropriate mixtures thereof. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis.

The methods and formulations described herein include the use of amorphous forms as well as crystalline forms (also known as polymorphs). Active metabolites of compounds or salts of any one of Formulas (I), (IA), (IB), (II), (III), (IIIA), (IIIB), (IIIC), (IIID), (IVA), (IVB), (IVC), (IVD), (IVE), (IVF), (IVG) and (IVH) having the same type of activity are included in the scope of the present disclosure. In addition, the compounds described herein can exist in unsolvated as well as solvated forms with pharmaceutically acceptable solvents such as water, ethanol, and the like. The solvated forms of the compounds and salts presented herein are also considered to be disclosed herein.

In certain embodiments, compounds or salts of the compounds of any one of Formulas (I), (IA), (IB), (II), (III), (IIIA), (IIIB), (IIIC), (IIID), (IVA), (IVB), (IVC), (IVD), (IVE), (IVF), (IVG) and (IVH) may be prodrugs, e.g., wherein a carboxylic acid present in the parent compound is presented as an ester. The term “prodrug” is intended to encompass compounds which, under physiologic conditions, are converted into pharmaceutical agents, i.e., parent compound, of the present disclosure. One method for making a prodrug is to include one or more selected moieties which are hydrolyzed under physiologic conditions to reveal the desired molecule. In certain embodiments, the prodrug is converted by an enzymatic activity of the host animal such as enzymatic activity in specific target cells in the host animal. For example, esters or carbonates (e.g., esters or carbonates of alcohols or carboxylic acids) are preferred prodrugs of the present disclosure.

Prodrugs are often useful because, in some situations, they may be easier to administer than the parent drug. They may, for instance, be bioavailable by oral administration whereas the parent is not. Prodrugs may help enhance the cell permeability of a compound relative to the parent drug. For example, the prodrug may have improved cell permeability over the parent compound. The prodrug may also have improved solubility in pharmaceutical formulations over the parent drug. In some embodiments, the design of a prodrug increases the lipophilicity of the pharmaceutical agent. In some embodiments, the design of a prodrug increases the effective water solubility. See, e.g., Fedorak et al., Am. J. Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A. C. S. Symposium Series; and Edward B. Roche, Bioreversible Carriers in Drug Design, American Pharmaceutical Association and Pergamon Press, 1987, all incorporated herein for such disclosure).

According to another embodiment, the present disclosure provides methods of producing the above-defined compounds. The compounds may be synthesized using conventional techniques. Advantageously, these compounds are conveniently synthesized from readily available starting materials. Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

### Pharmaceutical Formulations

Provided herein, in certain embodiments, are compositions comprising a therapeutically effective amount of any compound or salt of any one of Formulas (I), (IA), (B), (II), (III), (IIIA), (IIIB), (IIIC), (IIID), (IVA), (IVB), (IVC), (IVD), (IVE), (IVF), (IVG) and (IVH) (also referred to herein as “the pharmaceutical agent”). In certain embodiments, a pharmaceutical formulation may be used in any of the methods described herein.

In certain embodiments, a compound of any one of Formulas (I), (IA), (IB), (II), (III), (IIIA), (IIIB), (IIIC), (IIID), (IVA), (IVB), (IVC), (IVD), (IVE), (IVF), (IVG) and (IVH) is used for the treatment of an ophthalmic disorder such as cataracts or presbyopia. A formulation administered to the eye may be administered by injection, for example, by intravitreal or intracameral injection. A formulation administered to the eye may be administered topically, for example, with an ointment, cream, or eye drop.

In certain embodiments, compounds or salts of the disclosure may exhibit low aqueous solubility. For example, a compound or salt of any one of Formulas (I), (IA), (IB), (II), (III), (IIIA), (IIIB), (IIIC), (IIID), (IVA), (IVB), (IVC), (IVD), (IVE), (IVF), (IVG) and (IVH) may have an aqueous solubility of slightly soluble (100 to 1000 approximate volume mL of solvent need to dissolve 1 g of solute), very slightly soluble (1000 to 10,000 approximate volume mL of solvent need to dissolve 1 g of solute), or practically insoluble (greater than 10,000 approximate volume (mL) needed to dissolve 1 g of solute).

In certain embodiments, the compounds or salts of the disclosure with low aqueous solubility may preferentially be formulated as aqueous suspensions, such as microparticle or nanoparticle aqueous suspensions. In certain embodiments, aqueous suspensions of compounds or salts described herein permit the formulation of a suitable amount of a compound or salt in a small amount of liquid acceptable for administration by injection into an eye wherein the suitable amount of the compound or salt is only partially or not fully soluble in the small amount of liquid.

In other embodiments, the compounds or salts of the disclosure with low aqueous solubility may preferentially be formulated with an agent that enhances aqueous solubility. For example, in certain embodiments, the formulations of the disclosure are aqueous formulations for topical administration, wherein the aqueous formulation comprises a solubilizing agent, e.g., a β-cyclodextrin, to enhance solubility of a compound of salt of the disclosure.

In certain embodiments, formulations of the disclosure comprise a compound or salt of any one of Formulas (I), (IA), (IB), (II), (III), (IIIA), (IIIB), (IIIC), (IIID), (IVA), (IVB), (IVC), (IVD), (IVE), (IVF), (IVG) and (IVH), wherein the compound or salt is largely free of impurities, such as at least about 80 wt % pure, at least about 81% pure, at least about 82% pure, at least about 83% pure, at least about 84% pure, at least about 85% pure, at least about 86% pure, at least about 87% pure, at least about 88% pure, at least about 89% pure, at least about 90% pure, at least about 91% pure, at least about 92% pure, at least about 93% pure, at least about 94% pure, at least about 95% pure, at least about 96% pure, at least about 97% pure, at least about 98% pure, at least about 99% pure, at least about 99.1% pure, at least about 99.2% pure, at least about 99.3% pure, at least about 99.4% pure, at least about 99.5% pure, at least about 99.6% pure, at least about 99.7% pure, at least about 99.8% pure, or at least about 99.9% pure. In certain embodiments, formulations of the disclosure comprise a compound or salt of any one of Formulas (I), (IA), (IB), (II), (III), (IIIA), (IIIB), (IIIC), (IIID), (IVA), (IVB), (IVC), (IVD), (IVE), (IVF), (IVG) and (IVH), wherein the compound or salt is about 70% to about 99.99%, about 80% to about 99.9%, about 85% to about 99%, about 90% to about 99%, about 95% to about 99%, about 97% to about 99%, about 98% to about 99%, about 98% to about 99.9%, about 99% to about 99.99%, about 99.5% to about 99.99%, about 99.6% to about 99.99%, about 99.8 to about 99.99%, or about 99.9% to about 99.99% free of impurities.

In certain embodiments, formulations of the compounds and salts described herein, may be aqueous suspensions of the compound or salt of any one of Formulas (I), (IA), (IB), (II), (III), (IIIA), (IIIB), (IIIC), (IIID), (IVA), (IVB), (IVC), (IVD), (IVE), (IVF), (IVG) and (IVH). The compound or salt may be in the form of particles, e.g., microparticles or nanoparticles, in an oily or aqueous medium. In certain embodiments, a formulation for injection into an eye or for topical administration to the eye is in the form of an aqueous suspension. In preferred embodiments, a formulation for injection into an eye is in the form of an aqueous suspension.

Particles, e.g., microparticles or nanoparticles, of the disclosure may be formed through methods such as milling using equipment such as ball mills, fluid energy (jet) mills, cutter mills, hammer mills, vibration mills, and pin mills. In particular embodiments, the particles of the disclosure are prepared with a ball mill such as a Retsch® ball mill (www.retsch.com). Particles of the formulations described herein may be prepared with a fluid energy mill such as a Fluid Energy jet mill (www.fluidenergype.com).

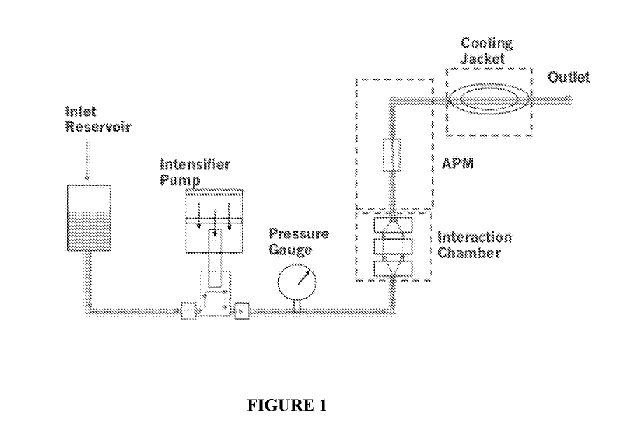

Microfluidization may be used to achieve uniform particle sizes and particle size reduction. A microfluidics system is depicted in FIG. 1. To perform particle size reduction, the product solution enters the microfluidics system through the inlet reservoir. The product solution is then powered by a high-pressure pump into auxiliary processing module (APM) and then to the interaction chamber. The product solution is then effectively cooled, if required, and collected in the outlet reservoir.

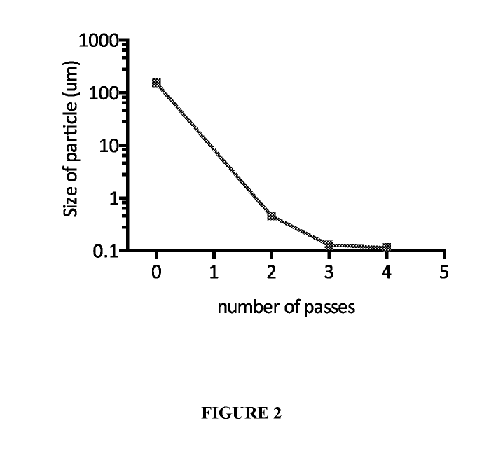

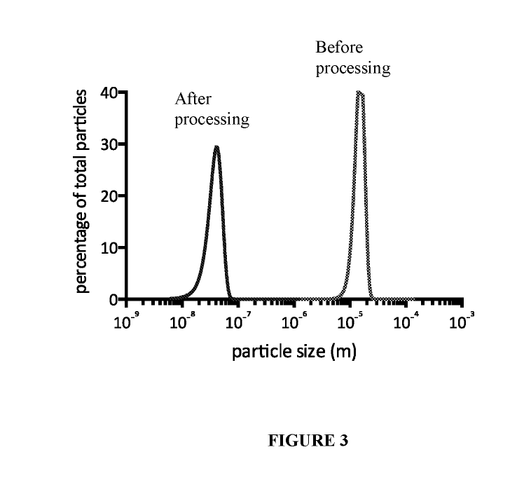

FIG. 2 shows that as the number of passes of the product solution through the microfluidics system increases, the size of the particles decreases. FIG. 3 shows that after processing of a product solution through a microfluidics chamber, the majority of the particles are found at smaller particle sizes.

In certain embodiments, particles of the formulations described herein have an average diameter from about 1 nm to about 10 μm, about 1 nm to about 10 μm, about 1 nm to about 5 μm, about 1 nm to about 2 μm, about 1 nm to about 1 μm, about 1 nm to about 900 nm, about 1 nm to about 800 nm, about 1 nm to about 700, about 1 nm to about 600 nm, about 1 nm to about 500 nm, about 1 nm to about 400 nm, about 1 nm to about 300 nm, about 1 nm to about 200 nm, or even from about 1 nm to about 100 nm. In certain embodiments, the average diameter is the average largest diameter or the average equivalent diameter.

In certain embodiments, greater than 80% of the particles, such as greater than 90% or greater than 95% of the particles in the formulation have an average largest particle diameter of from about 1 nm to about 10 μm, about 1 nm to about 10 μm, about 1 nm to about 5 μm, about 1 nm to about 2 μm, about 1 nm to about 1 μm, about 1 nm to about 900 nm, about 1 nm to about 800 nm, about 1 nm to about 700, about 1 nm to about 600 nm, about 1 nm to about 500 nm, about 1 nm to about 400 nm, about 1 nm to about 300 nm, about 1 nm to about 200 nm, or even from about 1 nm to about 100 nm. In certain embodiments, the average diameter is the average largest diameter or the average equivalent diameter.

In certain embodiments, particles of the formulations described herein have an average diameter from about 100 nm to about 10 μm, about 100 nm to about 10 μm, about 100 nm to about 5 μm, about 100 nm to about 2 μm, about 100 nm to about 1 μm, about 100 nm to about 900 nm, about 100 nm to about 800 nm, about 100 nm to about 700, about 100 nm to about 600 nm, about 200 nm to about 500 nm, about 250 nm to about 600 nm, about 300 nm to about 600 nm, about 350 nm to about 700 nm, about 450 nm to about 550 nm, about 475 nm to about 525 nm, or from about 400 nm to about 700 nm. In certain embodiments, the average diameter is the average largest diameter or the average equivalent diameter.

In certain embodiments, greater than 80% of the particles, such as greater than 90% or greater than 95% of the particles in the formulation have an average diameter from about 100 nm to about 10 μm, about 100 nm to about 10 μm, about 100 nm to about 5 μm, about 100 nm to about 2 μm, about 100 nm to about 1 μm, about 100 nm to about 900 nm, about 100 nm to about 800 nm, about 100 nm to about 700, about 100 nm to about 600 nm, about 200 nm to about 500 nm, about 250 nm to about 600 nm, about 300 nm to about 600 nm, about 350 nm to about 700 nm, about 450 nm to about 550 nm, about 475 nm to about 525 nm, or from about 400 nm to about 700 nm. In certain embodiments, the average diameter is the average largest diameter or the average equivalent diameter.

Measuring particle size can be done using a variety of techniques, including for example, light microscopy, scanning electron microscopy, or atomic force microscopy. Selecting a method for particle size measurement depends on the range needed, solubility, crystal structure, toxicity and flowability. Particle sizing methods can be grouped into three areas: (1) ensemble methods, where all particles in the sample are measured simultaneously, e.g., laser diffraction (LALLS—low angle laser light scattering) and dynamic light scattering (QELS—quasi-elastic light scattering); (2) counting methods, where individual particles are measured and divided into bins according to their size, e.g., electrozone counters (Coulter) and different types of microscopy; and (3) separation methods, where an outside process is used to separate particles according to size, e.g., sedimentation field-flow fractionation, and differential sedimentation using a disc centrifuge.

In certain embodiments, the particle measurement data is presented as a volume distribution or a weight distribution as a function of particle diameter. Distribution may be reported as the dispersity: D_{M}=M_{w}/Mₙ, where M_{w} is the mass-average molar mass (or molecular weight) and Mₙ is the number-average molar mass (or molecular weight). Pure Appl. Chem., 2009, 81(2), 351-353. Particle size distribution may be described in the format: D(50), which represents the average equivalent diameter where 50 mass % have larger equivalent diameter and 50 mass % have smaller equivalent diameter and the “equivalent diameter” refers to the particle diameter if the particle was spherical.

The amount of the compound or salt in a pharmaceutical formulation of the invention can be measured as a percentage of mass per volume. In certain embodiments, a formulation such as an aqueous suspension of the disclosure, comprises from about 0.05 wt % to about 10 wt % of the compound or salt of any one of Formulas (I), (IA), (IB), (II), (III), (IIIA), (IIIB), (IIIC), (IIID), (IVA), (IVB), (IVC), (IVD), (IVE), (IVF), (IVG) and (IVH). In certain embodiments, the formulation comprises from about 0.1 wt % to about 5 wt %, about 0.1 wt % to about 4 wt %, about 0.1 wt % to about 3 wt %, about 0.1 wt % to about 2 wt %, about 0.2 wt % to about 5 wt %, about 0.2 wt % to about 4 wt %, 0.2 wt % to about 3 wt %, about 0.3 wt % to about 5 wt %, about 0.3 wt % to about 4 wt %, or about 0.4 wt % to about 4 wt %, of a compound or salt described herein. In certain embodiments, the formulation comprises from about 1 wt % to about 5 wt %, about 1.5 wt % to about 5 wt %, about 1.5 wt % to about 4.5 wt %, about 2 wt % to about 4 wt %, about 2.5 wt % to about 3.5 wt %, or about 2.8 wt % to about 3.2 wt % of a compound or salt described herein.

In certain embodiments, a formulation such as an aqueous suspension of the disclosure, comprises about 0.01 wt %, about 0.02 wt %, about 0.03 wt %, about 0.04 wt %, about 0.05 wt %, about 0.06 wt %, about 0.07 wt %, about 0.08 wt %, about 0.09 wt %, about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1 wt %, about 1.1 wt %, about 1.2 wt %, about 1.3 wt %, about 1.4 wt %, about 1.5 wt %, about 1.6 wt %, about 1.7 wt %, about 1.8 wt %, about 1.9 wt %, about 2 wt %, about 2.1 wt %, about 2.2 wt %, about 2.3 wt %, about 2.4 wt %, about 2.5 wt %, about 2.6 wt %, about 2.7 wt %, about 2.8 wt %, about 2.9 wt %, about 3 wt %, about 3.1 wt %, about 3.2 wt %, about 3.3 wt %, about 3.4 wt %, about 3.5 wt %, about 3.6 wt %, about 3.7 wt %, about 3.8 wt %, about 3.9 wt %, about 4 wt %, about 4.1 wt %, about 4.2 wt %, about 4.3 wt %, about 4.4 wt %, about 4.5 wt %, about 5 wt %, about 6 wt %, about 7 wt %, about 8 wt %, about 9 wt %, or about 10 wt % of a compound or salt described herein.

A compound or salt described herein can be present in a formulation of the invention at a concentration of, for example, about 500 nM, about 600 nM, about 700 nM, about 800 nM, about 900 nM, about 1 μM, about 2 μM, about 3 μM, about 4 μM, about 5 μM, about 6 μM, about 7 μM, about 8 μM, about 9 μM, about 10 μM, about 20 μM, about 30 μM, about 40 μM, about 50 μM, about 60 μM, about 70 μM, about 80 μM, about 90 μM, about 100 μM, about 150 μM, about 200 μM, about 250 μM, about 300 μM, about 350 μM, about 400 μM, about 450 μM, about 500 μM, about 550 μM, about 600 μM, about 650 μM, about 700 μM, about 750 μM, about 800 μM, about 850 μM, about 900 μM, about 1 mM, about 5 mM, about 10 mM, about 15 mM, about 20 mM, about 25 mM, about 30 mM, about 35 mM, about 40 mM, about 45 mM, about 50 mM, about 55 mM, about 60 mM, about 65 mM, about 70 mM, about 75 mM, about 80 mM, about 85 mM, about 90 mM, about 95 mM, or about 100 mM. The compound described herein may be present in a composition within a range of concentrations, the range being defined by an upper and lower value selected from any of the preceding concentrations. For example, the compound or salt of the disclosure may be present in the formulation at a concentration of from about 1 nM to about 100 mM, about 10 nM to about 10 mM, about 100 nM to about 1 mM, about 500 nM to about 1 mM, about 1 mM to about 50 mM, about 10 mM to about 40 mM, about 20 mM to about 35 mM, or about 20 mM to about 30 mM.

In certain embodiments, an aqueous formulation of the disclosure comprises at least 90 wt % water, such as at least 91 wt %, at least 92 wt %, at least 93 wt %, at least 94 wt %, at least 95 wt %, at least 96 wt %, at least 97 wt %, at least 98 wt %, or even at least 99 wt % of water.

In certain embodiments, the pharmaceutical formulations can be in a form suitable for parenteral injection as a sterile suspension, solution, or emulsion in oily or aqueous vehicles, and can contain formulation agents such as suspending, stabilizing, and/or dispersing agents. Pharmaceutical formulations for parenteral administration include, for example, aqueous solutions of the active compounds in water-soluble form. Suspensions of the active compounds can be prepared, for example, as oily injection suspensions. Suitable lipophilic solvents or vehicles include fatty oils such as sesame oil, or synthetic fatty acid esters, such as ethyl oleate, isopropyl palmitate, or medium chain triglycerides, or liposomes. In preferred embodiments, a formulation for parenteral administration is an aqueous suspension.

In certain embodiments, a formulation, such as an aqueous suspension, of the compounds and salts of any one of Formulas (I), (IA), (IB), (II), (III), (IIIA), (IIIB), (IIIC), (IIID), (IVA), (IVB), (IVC), (IVD), (IVE), (IVF), (IVG) and (IVH) comprises an agent which increases the viscosity of the suspension, such as carboxymethyl cellulose (CMC), hydroxyethyl cellulose, polyethylene glycol (PEG), hydroxypropyl methyl cellulose (HPMC), sorbitol, gellan gum (high or low acyl), xanthan gum, dextran, guar gum, locust bean gum, sodium alginate, agar, gelatin, chitosan, pectin, alginates, xyloglucan, polyvinyl alcohol, polyvinyl pyrrolidone, carrageenan and combinations thereof. In certain embodiments, the agent that increases viscosity of the formulation is an in situ gel-forming agent wherein gel formation is triggered by temperature, pH, or ion concentration. Examples of in situ gel forming agents include poly(lactic-co-glycolic acid) (PLGA), poloxamers, xyloglucans, and poly(N-isopropyl acrylamide) polymers, alginates, gellan gum (low or high acyl), cellulose acetate or cellulose phthalate, and xanthan gum. In certain embodiments, a formulation, such as an aqueous suspension of the disclosure comprises from about 0.05 wt % to about 1 wt %, about 0.1 to about 0.8 wt %, about 0.1 wt % t to about 0.7 wt %, about 0.1 wt % to about 0.6 wt %, about 0.1 wt % to about 0.5 wt %, about 0.1 wt % to about 0.4 wt %, or even 0.1 wt % to about 0.3 wt %. In certain embodiments, an aqueous suspension for injection, e.g., intravitreal injection, comprises an agent which increases viscosity, such as gellan gum. In certain embodiments, an aqueous suspension for topical administration comprises an agent which increases viscosity, such as gellan gum.

In some embodiments, the addition of an excipient can change the viscosity of a pharmaceutical formulation of the invention. In some embodiments the use of an excipient can increase or decrease the viscosity of a fluid by at least 0.001 Pascal-second (Pa·s), at least 0.0009 Pa·s, at least 0.0008 Pa·s, at least 0.0007 Pa·s, at least 0.0006 Pa·s, at least 0.0005 Pa·s, at least 0.0004 Pa·s, at least 0.0003 Pa·s, at least 0.0002 Pa·s, at least 0.0001 Pa·s, at least 0.00005 Pa·s, or at least 0.00001 Pa·s. In some embodiments the use of an excipient can increase the viscosity of a fluid by about 0.00001 Pa·s and 0.01 Pa·s, between 0.00005 Pa·s and 0.005 Pa·s, between 0.0001 Pa·s and 0.001 Pa·s, between 0.0002 Pa·s and 0.001 Pa·s, between 0.0005 Pa·s and 0.0009 Pa·s, or between 0.0006 Pa·s and 0.0008 Pa·s. In some embodiments the use of an excipient can increase or decrease the viscosity of a fluid by at least 0.001 Pascal-second (Pa·s), at least 0.002 Pa·s, at least 0.004 Pa·s, at least 0.006 Pa·s, at least 0.008 Pa·s, at least 0.01 Pa·s, at least 0.012 Pa·s, at least 0.014 Pa·s, at least 0.016 Pa·s, at least 0.018 Pa·s, at least 0.02 Pa·s, at least 0.022 Pa·s, at least about 0.024 Pa·s, at least about 0.026 Pa·s, at least about 0.028 Pa·s, or at least about 0.03 Pa·s.

In certain embodiments, a formulation of the disclosure, such as a formulation for administration by injection or topical administration to the eye, has a viscosity of about 0.001 Pa·s to about 0.05 Pa·s, about 0.001 Pa·s to about 0.03 Pa·s, about 0.001 Pa·s to about 0.02 Pa·s, about 0.001 Pa·s to about 0.01 Pa·s, about 0.005 Pa·s to about 0.030 Pa·s, about 0.01 Pa·s to about 0.03 Pa·s, about 0.015 Pa·s to about 0.025 Pa·s.

In some embodiments, the addition of an excipient to a pharmaceutical formulation of the invention can increase or decrease the viscosity of the composition by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%. In some embodiments, the addition of an excipient to a pharmaceutical formulation of the invention can increase or decrease the viscosity of the composition by no greater than 5%, no greater than 10%, no greater than 15%, no greater than 20%, no greater than 25%, no greater than 30%, no greater than 35%, no greater than 40%, no greater than 45%, no greater than 50%, no greater than 55%, no greater than 60%, no greater than 65%, no greater than 70%, no greater than 75%, no greater than 80%, no greater than 85%, no greater than 90%, no greater than 95%, or no greater than 99%. Examples of ranges which the viscosity change falls within can be created by combining any two of the preceding percentages. For example the addition of an excipient can increase or decrease the viscosity of the composition by 5% to 99%, by 10% to 95%, by 20% to 70% or by 35% to 55%.

In certain embodiments, a formulation, such as an aqueous suspension, of the compounds and salts of any one of Formulas (I), (IA), (IB), (II), (III), (IIIA), (IIIB), (IIIC), (IIID), (IVA), (IVB), (IVC), (IVD), (IVE), (IVF), (IVG) and (IVH) comprises an agent for adjusting the pH of the formulation. In certain embodiments, the agent for adjusting the pH could be an acid, e.g., hydrochloric acid or boric acid, or a base, e.g., sodium hydroxide or potassium hydroxide. In certain embodiments, the agent for adjusting the pH is an acid such as boric acid. The formulation may comprise about 0.05 wt % to about 5 wt %, about 0.1% to about 4%, about 0.1% to about 3 wt %, about 0.1 wt % to about 2 wt %, or about 0.1 wt % to about 1 wt % of an agent for adjusting the pH.

Formulations of the disclosure can be formulated at any suitable pH. In certain embodiments, the pH of the formulation is about 4, about 4.05, about 4.1, about 4.15, about 4.2, about 4.25, about 4.3, about 4.35, about 4.4, about 4.45, about 4.5, about 4.55, about 4.6, about 4.65, about 4.7, about 4.75, about 4.8, about 4.85, about 4.9, about 4.95, about 5, about 5.1, about 5.2, about 5.3, about 5.4, about 5.5, about 5.6, about 5.7, about 5.8, about 5.9, about 6, about 6.1, about 6.2, about 6.3, about 6.4, about 6.5, about 6.6, about 6.7, about 6.8, about 6.9, about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, or about 9 pH units. In certain embodiments, the pH of the formulation is from about 4 to about 10, about 5 to about 9, about 6 to about 8, about 6.5 to about 8, about 7 to about 8, about 7.2 to about 8, about 7.2 to about 7.8, about 7.3 to about 7.5, or about 7.35 to about 7.45. In some embodiments the pH of the formulation is about 7.4.

In certain embodiments, a formulation, such as an aqueous suspension, of the compounds and salts of any one of Formulas (I), (IA), (IB), (II), (III), (IIIA), (IIIB), (IIIC), (IIID), (IVA), (IVB), (IVC), (IVD), (IVE), (IVF), (IVG) and (IVH) comprises a preservative agent. Examples of preservative agents include benzalkonium chloride, ethylenediaminetetraacetic acid (EDTA), chlorobutanol, phenylmercuric acetate, phenylmercuric nitrate, chlorhexidine acetate, thimerosal, and benzethonium chloride. In certain embodiments, a formulation of the disclosure comprises from about 0.001 wt % to about 1 wt %, about 0.001 wt % to about 0.5 wt %, about 0.001 wt % to about 0.1 wt %, about 0.001 wt % to about 0.05 wt %, about 0.001 wt % to about 0.01 wt %, about 0.001 wt % to about 0.005 wt % of a preservative agent. In certain embodiments, the formulation of the disclosure comprises benzalkonium chloride from about 0.0001 wt % to about 0.1 wt %, about 0.005 wt % to about 0.1 wt %, about 0.005 wt % to about 0.05 wt %, such as about 0.01 wt %.

In certain embodiments, a formulation of the disclosure does not include a preservative. In certain embodiments, an injectable formulation such as an intravitreal formulation does not include a preservative.

In certain embodiments, a formulation, such as an aqueous suspension, of the compounds and salts of any one of Formulas (I), (IA), (IB), (II), (III), (IIIA), (IIIB), (IIIC), (IIID), (IVA), (IVB), (IVC), (IVD), (IVE), (IVF), (IVG) and (IVH) further comprises one or more physiologically acceptable carriers including excipients and auxiliaries which facilitate processing of the pharmaceutical agent into preparations which are used pharmaceutically. Proper formulation is dependent upon the route of administration chosen. A summary of pharmaceutical formulations is found, for example, in Remington: The Science and Practice of Pharmacy, Nineteenth Ed (Easton, Pa., Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999).

In certain embodiments, a formulation, such as an aqueous suspension, of the compounds and salts of any one of Formulas (I), (IA), (IB), (II), (III), (IIIA), (IIIB), (IIIC), (IIID), (IVA), (IVB), (IVC), (IVD), (IVE), (IVF), (IVG) and (IVH) further comprises an agent for adjusting the osmolarity of the formulation, e.g., mannitol, sodium chloride, sodium sulfate, dextrose, potassium chloride, glycerin, propylene glycol, calcium chloride, and magnesium chloride. In certain embodiments, the formulation comprises from about 0.1 wt % to about 10 wt %, about 0.5 wt % to about 8 wt %, about 1 wt % to about 5 wt %, about 1 wt % to about 4 wt %, or about 1 wt % to about 3 wt % of an agent for adjusting the osmolarity of the formulation.

In certain embodiments, the formulation of the disclosure has an osmolarity from about 10 mOsm to about 1000 mOsm, about 100 mOsm to about 700 mOsm, about 200 mOsm to about 400 mOsm, about 250 mOsm to about 350 mOsm or even about 290 mOsm to about 310 mOsm.

In certain embodiments, a formulation, such as an aqueous suspension, of the compounds and salts of any one of Formulas (I), (IA), (IB), (II), (III), (IIIA), (IIIB), (IIIC), (IIID), (IVA), (IVB), (IVC), (IVD), (IVE), (IVF), (IVG) and (IVH) further comprises a buffering agent, such as tromethamine, potassium phosphate, sodium phosphate, saline sodium citrate buffer (SSC), acetate, saline, physiological saline, phosphate buffer saline (PBS), 4-2-hydroxyethyl-1-piperazineethanesulfonic acid buffer (HEPES), 3-(N-morpholino)propanesulfonic acid buffer (MOPS), and piperazine-N,N′-bis(2-ethanesulfonic acid) buffer (PIPES), sodium acetate-boric acid stock solution, boric acid-sodium carbonate with sodium chloride solution, boric acid-sodium borate buffer, sodium and potassium phosphate buffers, boric acid-sodium carbonate with potassium chloride, or combinations thereof. In certain embodiments, the formulation comprises from about 0.05 wt % to about 5 wt %, about 0.1 wt % to about 4 wt %, about 0.1 wt % to about 3 wt %, about 0.1 wt % to about 2 wt %, or about 0.1 wt % to about 1 wt % of an agent for buffering the formulation, e.g., tromethamine.

In certain embodiments, a formulation, such as an aqueous suspension, of the compounds and salts of any one of Formulas (I), (IA), (IB), (II), (III), (IIIA), (IIIB), (IIIC), (IIID), (IVA), (IVB), (IVC), (IVD), (IVE), (IVF), (IVG) and (IVH) further comprises a dispersion agent. Examples of dispersion agents include surfactants such as sorbitan ether esters of oleic acid, polysorbate-80, and polysorbate-20, cationic surfactants, and anionic surfactants. In preferred embodiments, the formulation comprises a dispersion agent that is a nonionic surfactant. In certain embodiments, the formulation comprises from about 0.01 wt % to about 1 wt %, about 0.02 wt % to about 1 wt %, about 0.02 wt % to about 0.8 wt %, about 0.5 wt % to about 0.5 wt %, or about 0.05 wt % to about 0.3 wt % of a dispersion agent, e.g., polysorbate-80.

In certain embodiments, a formulation, such as an aqueous solution, of the compounds and salts of any one of Formulas (I), (IA), (IB), (II), (III), (IIIA), (IIIB), (IIIC), (IIID), (IVA), (IVB), (IVC), (IVD), (IVE), (IVF), (IVG) and (IVH), further comprises a solubilizing agent. In certain embodiments, the compound or salt of the disclosure exhibits low aqueous solubility and the addition of a solubilizing agent enhances the solubility of the compound or salt. Solubilizing agents of the disclosure include, for example, host molecules of inclusions complexes such as cyclodextrins. In some embodiments β-cyclodextrins are preferred. An example of a suitable β-cyclodextrin includes, for example, (2-hydroxylpropyl)-β-cyclodextrin. In certain embodiments, the formulation comprises from about 2 wt % to about 15 wt % of a solubilizing agent, about 3 wt % to about 12 wt %, about 4 wt % to about 10 wt %, about 5 wt % to about 10 wt %, or about 6 wt % to about 10 wt % of a solubilizing agent, e.g., a cyclodextrin. In certain embodiments, the formulation is an aqueous solution comprising a solubilizing agent, such as a β-cyclodextrin. In preferred embodiments, a formulation for topical administration to the eye comprises a solubilizing agent such as a cyclodextrin.

In certain embodiments, the formulations of the disclosure may include one or more additional excipients described below. The amount of the excipient in a pharmaceutical formulation of the disclosure can be about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 6%, about 7%, about 8%, about 9%, about 10%, about 15%, about 20%, about 25%, about 30%, about 35%, about 40%, about 45%, about 50%, about 60%, about 70%, about 80%, about 90%, about 100%, about 200%, about 300%, about 400%, about 500%, about 600%, about 700%, about 800%, about 900%, or about 1000% by mass of the compound in the pharmaceutical formulation. The amount of the excipient in a pharmaceutical formulation of the disclosure can be between 0.01% and 1000%, between 0.02% and 500%, between 0.1% and 100%, between 1% and 50%, between 0.01% and 1%, between 1% and 10%, between 10% and 100%, between 50% and 150%, between 100% and 500%, or between 500% and 1000% by mass of the compound of the invention in the pharmaceutical formulation.

The amount of the excipient in a pharmaceutical formulation of the invention can be about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 6%, about 7%, about 8%, about 9%, about 10%, about 15%, about 20%, about 25%, about 30%, about 35%, about 40%, about 45%, about 50%, about 55% about 60%, about 65%, about 70%, about 75%, about 80%, about 85%, about 90%, about 95%, about 99%, or about 100% by mass or by volume of the unit dosage form. The amount of the excipient in a pharmaceutical formulation of the invention can be between 0.01% and 1000%, between 0.02% and 500%, between 0.1% and 100%, between 1% and 50%, between 0.01% and 1%, between 1% and 10%, between 10% and 100%, between 50% and 150%, between 100% and 500%, or between 500% and 1000% by mass or by volume of the unit dosage form.

The ratio of a compound of the invention to an excipient in a pharmaceutical formulation of the invention can be about 100:about 1, about 95:about 1, about 90:about 1, about 85:about 1, about 80:about 1, about 75:about 1, about 70:about 1, about 65:about 1, about 60:about 1, about 55:about 1, about 50:about 1, about 45:about 1, about 40:about 1, about 35:about 1 about 30:about 1, about 25:about 1, about 20:about 1, about 15:about 1, about 10:about 1, about 9:about 1, about 8:about 1, about 7:about 1, about 6:about 1, about 5:about 1, about 4:about 1, about 3:about 1, about 2:about 1, about 1:about 1, about 1:about 2, about 1:about 3, about 1:about 4, about 1:about 5, about 1:about 6, about 1:about 7, about 1:about 8, about 1:about 9, or about 1:about 10. The ratio of a compound of the invention to an excipient in a pharmaceutical formulation of the invention can be within the range of between about 100:about 1 and about 1 to about 10, between about 10:about 1 and about 1:about 1, between about 5:about 1 and about 2:about 1.

Pharmaceutically acceptable carriers are well known in the art and include, for example, aqueous solutions such as water or physiologically buffered saline or other solvents or vehicles such as glycols, glycerol, oils such as olive oil, or injectable organic esters. In a preferred embodiment, when such pharmaceutical formulations are for human administration, particularly for invasive routes of administration, e.g., routes, such as injection or implantation, that circumvent transport or diffusion through an epithelial barrier, the aqueous solution is pyrogen-free, or substantially pyrogen-free. The excipients can be chosen, for example, to effect delayed release of an agent or to selectively target one or more cells, tissues or organs. The pharmaceutical formulation can be in dosage unit form such as tablet, capsule, granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

Methods for the preparation of compositions comprising the compounds described herein can include formulating the compounds with one or more inert, pharmaceutically-acceptable excipients. Liquid compositions include, for example, solutions in which a compound is dissolved, emulsions comprising a compound, or a solution containing liposomes, micelles, or nanoparticles comprising a compound as disclosed herein. These compositions can also contain minor amounts of nontoxic, auxiliary substances, such as wetting or emulsifying agents, pH buffering agents, and other pharmaceutically-acceptable additives.

Non-limiting examples of dosage forms suitable for use in the disclosure include liquid, elixir, nanosuspension, microsuspension, aqueous or oily suspensions, drops, syrups, and any combination thereof. Non-limiting examples of pharmaceutically-acceptable excipients suitable for use in the disclosure include granulating agents, binding agents, lubricating agents, disintegrating agents, anti-adherents, anti-static agents, surfactants, anti-oxidants, coloring agents, flavouring agents, plasticizers, preservatives, suspending agents, emulsifying agents, plant cellulosic material and spheronization agents, and any combination thereof. Non-limiting examples of types of formulations that can be used in a method of the invention include an aqueous solution, an ointment, an aqueous suspension, and an oil in water emulsion.

Pharmaceutical formulations suitable for oral administration can be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient can be presented as a bolus, electuary or paste.

Pharmaceutical formulations which can be used orally include tablets, push-fit capsules made of gelatin, as well as soft, sealed capsules made of gelatin and a plasticizer, such as glycerol or sorbitol. Tablets can be made by compression or molding, optionally with one or more accessory ingredients. Compressed tablets can be prepared by compressing in a suitable machine the active ingredient in a free-flowing form such as a powder or granules, optionally mixed with binders, inert diluents, or lubricating, surface active or dispersing agents. Molded tablets can be made by molding in a suitable machine a mixture of the powdered compound moistened with an inert liquid diluent. In some embodiments, the tablets are coated or scored and are formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or Dragee coatings for identification or to characterize different combinations of active compound doses.

Formulations for injection can be presented in unit dosage form, for example, in ampoules or in multi-dose containers, with an added preservative. The compositions can take such forms as suspensions, solutions or emulsions in oily or aqueous vehicles, and can contain formulatory agents such as suspending, stabilizing and/or dispersing agents. The compositions can be presented in unit-dose or multi-dose containers, for example sealed ampoules and vials, and can be stored in powder form or in a freeze-dried (lyophilized) condition requiring only the addition of the sterile liquid carrier, for example, saline or sterile pyrogen-free water, immediately prior to use. Extemporaneous injection solutions and suspensions can be prepared from sterile powders, granules and tablets of the kind previously described.

Pharmaceutical formulations for parenteral administration include aqueous and non-aqueous (oily) sterile injection solutions of the active compounds which can contain antioxidants, buffers, bacteriostats and solutes which render the formulation isotonic with the blood of the intended recipient; and aqueous and non-aqueous sterile suspensions which can include suspending agents and thickening agents. Suitable lipophilic solvents or vehicles include fatty oils such as sesame oil, or synthetic fatty acid esters, such as ethyl oleate or triglycerides, or liposomes.

Pharmaceutical formulations can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example, as an emulsion in an acceptable oil) or ion exchange resins, or as sparingly soluble derivatives, for example, as a sparingly soluble salt.

For buccal or sublingual administration, the compositions can take the form of tablets, lozenges, pastilles, or gels formulated in conventional manner. Such compositions can comprise the active ingredient in a flavored basis such as sucrose and acacia or tragacanth.

Pharmaceutical formulations can also be formulated in rectal compositions such as suppositories or retention enemas, e.g., containing conventional suppository bases such as cocoa butter, polyethylene glycol, or other glycerides.

Pharmaceutical formulations can be administered topically, that is by non-systemic administration. This includes the application of a compound of the present invention externally to the epidermis or the buccal cavity and the instillation of such a compound into the ear, eye and nose, such that the compound does not significantly enter the blood stream.

In contrast, systemic administration refers to oral, intravenous, intraperitoneal and intramuscular administration.

Pharmaceutical formulations suitable for topical administration include liquid or semi-liquid preparations suitable for penetration through the skin to the site of inflammation such as gels, liniments, lotions, creams, ointments or pastes, and drops suitable for administration to the eye, ear or nose.

Pharmaceutical formulations for administration by inhalation are conveniently delivered from an insufflator, nebulizer pressurized packs or other convenient means of delivering an aerosol spray. Pressurized packs can comprise a suitable propellant such as dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Alternatively, for administration by inhalation or insufflation, pharmaceutical preparations can take the form of a dry powder composition, for example a powder mix of the compound and a suitable powder base such as lactose or starch. The powder composition can be presented in unit dosage form, in for example, capsules, cartridges, gelatin or blister packs from which the powder can be administered with the aid of an inhalator or insufflator.

In some embodiments, the pharmaceutical formulation provided herein comprises a sugar as an excipient. Non-limiting examples of sugars include trehalose, sucrose, glucose, lactose, galactose, glyceraldehyde, fructose, dextrose, maltose, xylose, mannose, maltodextrin, starch, cellulose, lactulose, cellobiose, mannobiose, and combinations thereof.

In some embodiments, a pharmaceutical formulation of the invention comprises a source of divalent metal ions as an excipient. A metal can be in elemental form, a metal atom, or a metal ion. Non-limiting examples of metals include transition metals, main group metals, and metals of Group 1, Group 2, Group 3, Group 4, Group 5, Group 6, Group 7, Group 8, Group 9, Group 10, Group 11, Group 12, Group 13, Group 14, and Group 15 of the Periodic Table. Non-limiting examples of metals include lithium, sodium, potassium, cesium, magnesium, calcium, strontium, scandium, titanium, vanadium, chromium, manganese, iron, cobalt, nickel, copper, zinc, yttrium, zirconium, niobium, molybdenum, palladium, silver, cadmium, tungsten, rhenium, osmium, iridium, platinum, gold, mercury, cerium, and samarium.

In some embodiments, the pharmaceutical formulation provided herein comprises an alcohol as an excipient. Non-limiting examples of alcohols include ethanol, propylene glycol, glycerol, polyethylene glycol, chlorobutanol, isopropanol, xylitol, sorbitol, maltitol, erythritol, threitol, arabitol, ribitol, mannitol, galactilol, fucitol, lactitol, and combinations thereof.

Pharmaceutical preparations can be formulated with polyethylene glycol (PEG). PEGs with molecular weights ranging from about 300 g/mol to about 10,000,000 g/mol can be used. Non-limiting examples of PEGs include PEG 200, PEG 300, PEG 400, PEG 540, PEG 550, PEG 600, PEG 1000, PEG 1450, PEG 1500, PEG 2000, PEG 3000, PEG 3350, PEG 4000, PEG 4600, PEG 6000, PEG 8000, PEG 10,000, and PEG 20,000.

Further excipients that can be used in a composition of the invention include, for example, benzalkonium chloride, benzethonium chloride, benzyl alcohol, butylated hydroxyanisole, butylated hydroxytoluene, dehydroacetic acid, ethylenediamine, ethyl vanillin, glycerin, hypophosphorous acid, phenol, phenylethyl alcohol, phenylmercuric nitrate, potassium benzoate, potassium metabisulfite, potassium sorbate, sodium bisulfite, sodium metabisulfite, sorbic acid, thimerasol, acetic acid, aluminum monostearate, boric acid, calcium hydroxide, calcium stearate, calcium sulfate, calcium tetrachloride, cellulose acetate pthalate, microcrystalline celluose, chloroform, citric acid, edetic acid, and ethylcellulose.

In some embodiments, the pharmaceutical formulation provided herein comprises an aprotic solvent as an excipient. Non-limiting examples of aprotic solvents include perfluorohexane, α,α,α-trifluorotoluene, pentane, hexane, cyclohexane, methylcyclohexane, decalin, dioxane, carbon tetrachloride, freon-11, benzene, toluene, carbon disulfide, diisopropyl ether, diethyl ether, t-butyl methyl ether, ethyl acetate, 1,2-dimethoxyethane, 2-methoxyethyl ether, tetrahydrofuran, methylene chloride, pyridine, 2-butanone, acetone, N-methylpyrrolidinone, nitromethane, dimethylformamide, acetonitrile, sulfolane, dimethyl sulfoxide, and propylene carbonate.

Compositions of the invention can be packaged as a kit. In some embodiments, a kit includes written instructions on the administration or use of the composition. The written material can be, for example, a label. The written material can suggest conditions methods of administration. The instructions provide the subject and the supervising physician with the best guidance for achieving the optimal clinical outcome from the administration of the therapy. In some embodiments, the label can be approved by a regulatory agency, for example the U.S. Food and Drug Administration (FDA), the European Medicines Agency (EMA), or other regulatory agencies.

### Dosing

The compositions and methods of the present disclosure may be utilized to treat an individual in need thereof. In certain embodiments, the individual is a mammal such as a human, or a non-human mammal. When administered to an animal, such as a human, the composition or the pharmaceutical agent, is preferably administered as a pharmaceutical formulation comprising, for example, a compound or salt of any one of Formulas (I), (IA), (IB), (II), (III), (IIIA), (IIIB), (IIIC), (IIID), (IVA), (IVB), (IVC), (IVD), (IVE), (IVF), (IVG) and (IVH) and a pharmaceutically acceptable carrier or excipient.

In practicing the methods of treatment or use provided herein, therapeutically-effective amounts of the compounds or salts described herein are administered in pharmaceutical formulations to a subject having a disease or condition to be treated. A therapeutically-effective amount can vary widely depending on the severity of the disease, the age and relative health of the subject, the potency of the compounds used, and other factors. Subjects can be, for example, human subjects such as elderly adults, adults, adolescents, pre-adolescents, children, toddlers, infants, or neonates. A subject can be a patient. Subjects can be non-human animals, for example, chimpanzees, apes, monkeys, cattle, horses, sheep, goats, swine, rabbits, dogs, and cats, rats, mice and guinea pigs.

In certain embodiments, formulations of the disclosure are used to treat ophthalmic diseases through administration to an eye of a subject. The formulations can be delivered to the eye topically through cream, ointment or liquid drop formulation. The formulation can be delivered to the eye through injection. Injectable solutions can be delivered directly into the anterior chamber, sclera, vitreous humor, cornea, crystalline lens, or surrounding tissue. The compositions can also be delivered as an intraocular perfusate.

Additionally, the compounds described herein can be administered to the eye through an implant or contact lens. The contact lens can be pretreated with the compounds described herein. The contact lens can also be provided in a kit containing components to prepare a coated lens, which can be provided as a dry powder formulation to be reconstituted or as a concentrated or ready to use solution. The compositions for these kits can be for single- or multi-use.

The compounds provided herein can also be administered to the eye through an ophthalmic rod or through an intraocular lens-hydrogel assembly.

In certain embodiments, a formulation such as an aqueous suspension of the disclosure, is administered by injection, e.g., intravitreal injection, into the eye. A dosage for a formulation of the compounds and salts described herein administered by intravitreal injection into an eye is preferably administered in a low total volume, such as less than about 200 μL, less than about 180 μL, less than about 160 μL, less than about 140 μL, less than about 120 μL, less than about 110 μL, less than about 100 μL, less than about 90 μL, less than about 80 μL, less than about 70 μL, less than about 60 μL or even less than about less than about 50 μL. In certain embodiments, a formulation of the disclosure administered by injection into the eye has a total volume of from about 50 μL to about 150 μL, from about 60 L to about 120 μL, such as a volume of about 100 μL. As understood to the person of skill in the art, adjustments to the volume may be made based upon factors such as the species of the subject, age of subject, etc.

A dosage for a formulation of the compounds and salts described herein administered by intracameral injection into an eye is preferably administered in a low total volume, such as less than about 40 μL, less than about 35 μL, less than about 30 μL, less than about 25 μL, less than about 20 μL, less than about 15 μL, less than about 10 μL, or less than about 5 μL. In certain embodiments, a formulation of the disclosure administered by intracameral injection into the eye has a total volume of from about 5 μL to about 50 μL, from about 5 μL to about 25 μL, such as a volume of about 5 μL to about 15 μL.

A dosage for a formulation of the compounds and salts described herein administered topically to an eye is preferably administered in an amount from about 5 μL to about 80 μL, about 5 μL to about 75 μL, about 5 μL to about 70 μL, about 5 μL to about 65 μL, about 5 μL to about 60 μL, about 5 μL to about 55 μL, about 5 μL to about 50 μL, about 10 μL to about 60 μL, or about 10 μL to about 50 μL.

In certain embodiments, a formulation of the disclosure is administered in a dose of about 5 μL, about 10 μL, about 15 μL, about 20 μL, about 25 μL, about 30 μL, about 35 μL, about 40 μL, about 45 μL, about 50 μL, about 60 μL, about 70 μL, about 80 μL, about 90 μL, about 100 μL, about 110 μL, about 120 μL, about 130 μL, about 140 μL, about 150 μL, about 160 μL, about 170 μL, about 180 μL, about 190 μL, about 200 μL, about 250 μL, about 300 μL, about 350 μL, about 400 μL, about 450 μL, about 500 μL, about 600 μL, about 700 μL, about 800 μL, about 900 μL, or about 1 mL.

In certain embodiments, formulation of the disclosure is administered in a dose comprising a compound or salt described herein in an amount of about 25 μg, about 30 μg, about 35 μg, about 40 μg, about 45 μg, about 50 μg, about 60 μg, about 70 μg, about 80 μg, about 90 μg, about 100 μg, about 125 μg, about 150 μg, about 175 μg, about 200 μg, about 250 μg, about 300 μg, about 350 μg, about 400 μg, about 450 μg, about 500 μg, about 600 μg, about 700 μg, about 800 μg, about 900 μg, about 1 mg, about 2 mg, about 3 mg, about 4 mg, about 5 mg, about 6 mg, about 7 mg, about 8 mg, about 9 mg, about 10 mg, or about 20 mg. In certain embodiments, a dose of a formulation described herein comprises a compound or salt of the disclosure in an amount of about 1 mg to about 10 mg, about 10 ug to about 10 mg, about 100 ug to about 5 mg, about 50 ug to about 5 mg, about 500 ug to about 4 mg, or about 1 mg to about 4 mg. In certain embodiments, an intravitreal dose comprises from about 0.1 mg to about 5 mg, or about 0.5 mg to about 3 mg of a compound or salt described herein.

Pharmaceutical formulations of the disclosure can be administered either acutely or chronically. Pharmaceutical formulations of the invention can be administered as a single treatment or as a course of treatment. Treatments can be applied once per day, twice per day (b.i.d.), three times per day, in the morning, in the evening, before sleeping, or continuously throughout the day. Treatments can be applied every day, every other day, every three days, twice weekly, once weekly, every other week, monthly, every six weeks, every other month, every three months, every six months, annually, every other year, every 5 years, or as required.

In certain embodiments, pharmaceutical formulations of the disclosure administered by intravitreal injection, may be administered once a week, once every other week, once a month, once every six weeks, once every other month, once every three months, once every six months, once annually, once every other year, once every 5 years, or as required.

In certain embodiments, pharmaceutical formulations of the disclosure administered topically to an eye, may be administered once, twice or three times daily, wherein the treatment may be administered daily for up to two weeks, up to six weeks, up to eight weeks, up to three months, up to six months or even up to a year.

In certain embodiments wherein a patient's status does improve, the dose of drug being administered is temporarily reduced or temporarily suspended for a certain length of time. In certain embodiments, the patient will have a drug holiday wherein the patient does not receive the drug or receives a reduced amount of the drug for a period of time. A drug holiday can be, for example, between 2 days and 1 year, including by way of example only, 2 days, 3 days, 4 days, 5 days, 6 days, 7 days, 10 days, 12 days, 15 days, 20 days, 28 days, or more than 28 days. A drug holiday may be for about 1 month, about 2 months, about 3 months, about 4 months, about 5 months, about 6 months, about 7 months, about 8 months, about 9 months, about 10 months, about 11 months or about 12 months. The dose reduction during a drug holiday can be, for example, by 10%-100% of the original administered dose, including by way of example only 10%, 15%, 20%, 25%, 30%, 35%, 40%, 45%, 50%, 55%, 60%, 65%, 70%, 75%, 80%, 85%, 90%, 95%, and 100%. For further examples the dose reduction can be between 10% and 100%, between 20% and 80%, between 30% and 70%, between 50% and 90%, between 80% and 100% or between 90% and 100%.

Once improvement of the patient's conditions has occurred, a maintenance dose can be administered if necessary. Subsequently, the dosage or the frequency of administration, or both, can be reduced, as a function of the symptoms, to a level at which the improved disease, disorder or condition can be retained.

Additional methods for administering the formulations described herein include, for example, limited to delivery via enteral routes including oral, gastric or duodenal feeding tube, rectal suppository, rectal enema, parenteral routes, injection, infusion, intraarterial, intracardiac, intradermal, intraduodenal, intramedullary, intramuscular, intraosseous, intraperitoneal, intrathecal, intravascular, intravenous, intravitreal, intracameral, epidural, subcutaneous, inhalational, transdermal, transmucosal, sublingual, buccal, topical, epicutaneous, dermal, enemaear drops, intranasal, and vaginal administration. The compounds described herein can be administered locally to the area in need of treatment, by for example, local infusion during surgery, topical application such as creams or ointments, injection, catheter, or implant. The administration can also be by direct injection at the site of a diseased tissue or organ.

A dose can be modulated to achieve a desired pharmacokinetic or pharmacodynamics profile, such as a desired or effective ocular tissue profile, as described herein.

Pharmacokinetic and pharmacodynamic data can be obtained by various experimental techniques. Appropriate pharmacokinetic and pharmacodynamic profile components describing a particular composition can vary due to variations in drug metabolism in human subjects. Pharmacokinetic and pharmacodynamic profiles can be based on the determination of the mean parameters of a group of subjects. The group of subjects includes any reasonable number of subjects suitable for determining a representative mean, for example, 5 subjects, 10 subjects, 15 subjects, 20 subjects, 25 subjects, 30 subjects, 35 subjects, or more. The mean is determined, for example, by calculating the average of all subject's measurements for each parameter measured. A dose can be modulated to achieve a desired pharmacokinetic or pharmacodynamics profile, such as a desired or effective ocular tissue, as described herein.

The pharmacodynamic parameters can be any parameters suitable for describing compositions of the invention. For example, the pharmacodynamic profile can be obtained at a time after dosing of, for example, about 1 hour, about 1.5 hours, about 2 hours, about 2.5 hours, about 3 hours, about 3.5 hours, about 4 hours, about 4.5 hours, about 5 hours, about 5.5 hours, about 6 hours, about 6.5 hours, about 7 hours, about 7.5 hours, about 8 hours, about 8.5 hours, about 9 hours, about 9.5 hours, about 10 hours, about 10.5 hours, about 11 hours, about 11.5 hours, about 12 hours, about 12.5 hours, about 13 hours, about 13.5 hours, about 14 hours, about 14.5 hours, about 15 hours, about 15.5 hours, about 16 hours, about 16.5 hours, about 17 hours, about 17.5 hours, about 18 hours, about 18.5 hours, about 19 hours, about 19.5 hours, about 20 hours, about 20.5 hours, about 21 hours, about 21.5 hours, about 22 hours, about 22.5 hours, about 23 hours, about 23.5 hours, about 24 hours, about 25 hours, about 26 hours, about 27 hours, about 28 hours, about 29 hours, about 30 hours, about 36 hours, about 42 hours, about 48 hours, about 54 hours, about 60 hours, about one week, about two weeks, about three weeks, or about one month. A range of after dosing times for determining the pharmacodynamic profile can be defined by selecting any two values from the preceding list as the maximum and minimum after dosing times. For example a pharmacodynamic profile can be determined between one minute and one month after dosing, between one hour and one week after dosing, or between 12 hours and 1 day after dosing.

The pharmacokinetic parameters can be any parameters suitable for describing a compound. The exposure of the compound in various tissues of the eye can be, for example, about 1 nM, about 2 nM, about 3 nM, about 4 nM, about 5 nM, about 6 nM, about 7 nM, about 8 nM, about 9 nM, about 10 nM, about 15 nM, about 20 nM, about 25 nM, about 30 nM, about 35 nM, about 40 nM, about 45 nM, about 50 nM, about 55 nM, about 60 nM, about 65 nM, about 70 nM, about 75 nM, about 80 nM, about 85 nM, about 90 nM, about 95 nM, about 100 nM, about 110 nM, about 120 nM, about 130 nM, about 140 nM, about 150 nM, about 160 nM, about 170 nM, about 180 nM, about 190 nM, about 200 nM, about 250 nM, about 300 nM, about 350 nM, about 350 nM, about 400 nM, about 450 nM, about 500 nM, about 550 nM, about 600 nM, about 650 nM, about 700 nM, about 750 nM, about 800 nM, about 850 nM, about 900 nM, about 950 nM, about 1 μM, about 2 μM, about 3 μM, about 4 μM, about 5 μM, about 6 μM, about 7 μM, about 8 μM, about 9 μM, about 10 μM, about 15 μM, about 20 μM, about 25 μM, about 30 μM, about 35 μM, about 40 μM, about 45 μM, about 50 μM, about 55 μM, about 60 μM, about 70 μM, about 80 μM, about 90 μM, or about 100 μM. A range of exposures of the compound in the tissues of the eye can be defined by selecting any two values from the preceding list. For example the exposure in the tissue may be between 1 nM and 100 uM, between 10 nM and 10 uM, between 100 nM and 1 uM or between 1 uM and 10 uM.

Bioanalytical methods can be used to measure the effective concentration of administered compound. The level of compound can be measured in the plasma or in the eye. Within the eye local concentrations of the compound can be measured in the lens, the cornea, the retina, the ciliary body, the ciliary processes, the vitreous humor or the aqueous humor. The concentration of the compound can be measured after a single treatment, either topical or injected. The concentration of the compound can be measured after administration b.i.d. for 2 days, for 3 days, for 4 days, for 5 days, for 6 days, for 7 days, for 8 days, for 9 days, for 10 days, for 12 days, for 14 days, for 3 weeks, for 4 weeks, for 2 months, or for 3 months. The concentration of the compound can be measured after b.i.d. administration for 3 to 5 days, for 3 to 7 days, for 7 to 14 days, for 7 to 21 days, for 14 to 21 days, for 2 to 5 weeks, for 1 to 2 months, for 1 to 4 months, for 1 to 6 months, for 6 to 12 months or after b.i.d. administration for more than 12 months. The concentration of the compound can be measured after b.i.d. administration for more than 3 days, for more than 1 week, for more than 2 weeks, for more than 1 month, or for more than 3 months.

The concentration of the compound can be measured after once daily administration for 2 days, for 3 days, for 4 days, for 5 days, for 6 days, for 7 days, for 8 days, for 9 days, for 10 days, for 12 days, for 14 days, for 3 weeks, for 4 weeks, for 2 months, or for 3 months. The concentration of the compound can be measured after once daily administration for 3 to 5 days, for 3 to 7 days, for 7 to 14 days, for 7 to 21 days, for 14 to 21 days, for 2 to 5 weeks, for 1 to 2 months, for 1 to 4 months, for 1 to 6 months, for 6 to 12 months or after once daily administration for more than 12 months. The concentration of the compound can be measured after once daily administration for more than 3 days, for more than 1 week, for more than 2 weeks, for more than 1 month, or for more than 3 months.

The compounds of this disclosure may be administered to achieve a ciliary process concentration of 1 nM to 1000 nM, 15 nM to 400 nM, 50 nM to 200 nM, 100 nM to 150 nM, 10 nM to 50 nM, or 10 nM to 20 nM. The compounds of this disclosure may be administered to achieve a corneal concentration of 10 nM to 500,000 nM, 100 nM to 100,000 nM, 200 nM to 50,000 nM, 200 nM to 20,000 nM, 1000 nM to 10,000 nM, 200 nM to 800 nM, 400 nM to 800 nM, 200 nM to 20,000 nM, or 200 nM to 15,000 nM. The compounds of this disclosure may be administered to achieve a retinal concentration of 10 nM to 100,000 nM, 50 nM to 50,000 nM, 100 nM to 50,000 nM, 500 nM to 50,000 nM, 500 nM to 40,000 nM, 500 nM to 30,000 nM, 100 nM to 10,000 nM, 200 nM to 5,000 nM, 200 nM to 3,000 nM, 200 nM to 2,000 nM, 10 nM to 100 nM, or 10 nM to 50 nM. The compounds of this disclosure may be administered to achieve a lens concentration of about 1 nM to about 100 uM, about 10 nM to about 50 uM, about 100 nM to about 20 uM, about 500 nM to about 10 uM, about 2 uM to about 10 uM, or about 5 uM to about 10 uM. The compounds of this disclosure may be administered to achieve a vitreous humor concentration of about 1 nM to about 100 uM, about 10 nM to about 50 uM, about 100 nM to about 20 uM, about 500 nM to about 10 uM, about 1 uM to about 10 uM, or about 1 uM to about 5 uM. The compounds of this disclosure may be administered to achieve an iris concentration of about 1 nM to about 100 uM, about 10 nM to about 50 uM, about 100 nM to about 10 uM, about 500 nM to about 5 uM, about 700 nM to about 3 uM, or about 1 uM to about 2 uM.

The Tₘₐₓ of a compound described herein can be, for example, not greater than about 0.1 hours, about 0.2 hours, about 0.3 hours, about 0.4 hours, about 0.5 hours, not greater than about 1 hours, not greater than about 1.5 hours, not greater than about 2 hours, not greater than about 2.5 hours, not greater than about 3 hours, not greater than about 3.5 hours, not greater than about 4 hours, not greater than about 4.5 hours, not greater than about 5 hours, or any other Tₘₐₓ appropriate for describing a pharmacokinetic profile of a compound described herein. The Tₘₐₓ can be, for example, about 0.1 hours to about 24 hours; about 0.1 hours to about 0.5 hours; about 0.5 hours to about 1 hour; about 1 hour to about 1.5 hours; about 1.5 hours to about 2 hour; about 2 hours to about 2.5 hours; about 2.5 hours to about 3 hours; about 3 hours to about 3.5 hours; about 3.5 hours to about 4 hours; about 4 hours to about 4.5 hours; about 4.5 hours to about 5 hours; about 5 hours to about 5.5 hours; about 5.5 hours to about 6 hours; about 6 hours to about 6.5 hours; about 6.5 hours to about 7 hours; about 7 hours to about 7.5 hours; about 7.5 hours to about 8 hours; about 8 hours to about 8.5 hours; about 8.5 hours to about 9 hours; about 9 hours to about 9.5 hours; about 9.5 hours to about 10 hours; about 10 hours to about 10.5 hours; about 10.5 hours to about 11 hours; about 11 hours to about 11.5 hours; about 11.5 hours to about 12 hours; about 12 hours to about 12.5 hours; about 12.5 hours to about 13 hours; about 13 hours to about 13.5 hours; about 13.5 hours to about 14 hours; about 14 hours to about 14.5 hours; about 14.5 hours to about 15 hours; about 15 hours to about 15.5 hours; about 15.5 hours to about 16 hours; about 16 hours to about 16.5 hours; about 16.5 hours to about 17 hours; about 17 hours to about 17.5 hours; about 17.5 hours to about 18 hours; about 18 hours to about 18.5 hours; about 18.5 hours to about 19 hours; about 19 hours to about 19.5 hours; about 19.5 hours to about 20 hours; about 20 hours to about 20.5 hours; about 20.5 hours to about 21 hours; about 21 hours to about 21.5 hours; about 21.5 hours to about 22 hours; about 22 hours to about 22.5 hours; about 22.5 hours to about 23 hours; about 23 hours to about 23.5 hours; or about 23.5 hours to about 24 hours. In certain embodiments, the Tₘₐₓ following injection is about 24 hours to about 48 hours or about 48 hours to about 72 hours after administration of the compound or salt. In certain embodiments, the Tₘₐₓ in the lens following intravitreal injection is about 24 hours to about 48 hours or about 48 hours to about 72 hours after administration of the compound or salt. In certain embodiments, the Tₘₐₓ for the retina may be many hours or even days following intravitreal administration of a formulation described herein.

### Methods of the Disclosure

The disclosure provides compounds and formulations for use in reducing or preventing alpha-crystallin protein aggregation. The aggregation of alpha-crystallin has been implicated in a variety of diseases of which the compounds and formulations described herein may be used to treat or prevent. Such diseases include, for example, cataracts, nuclear sclerosis, presbyopia, neurological diseases, Alexander disease, Creutzfeldt-Jacob disease, Alzheimer's disease, and Parkinson's disease.

In certain embodiments, the methods provided herein can be used to treat a disease or a condition that would benefit from reducing the likelihood of or reversing the aggregation of alpha-crystallin. The compounds or salts disclosed herein can be used as pharmacological chaperones of alpha-crystallin.

The methods provided herein can be used to treat, for example, a vision disorder such as cataract, age-related cataract, diabetic cataract, a cataract associated with surgery, a cataract resulting from radiation, a cataract resulting from a genetic illness, a cataract resulting from an infection, a cataract resulting from medication, or a hereditary form of cataract with early onset.

Vision disorders, as discussed herein, refer to disordered vision that may be associated with aberrant aggregation of crystallin proteins in the lens of the eye. The aberrant aggregation of crystallin proteins may be the primary factor resulting in the vision disorder or may be one of a plurality of mechanisms resulting in the vision disorder. Vision disorders of the disclosure include, but are not limited to, cataract, such as nuclear cataract, cortical cataract, posterior capsular cataract, congenital cataract, early-onset hereditary cataract, metabolic (diabetic) cataract, secondary cataract, blunt traumatic cataract, penetrating traumatic cataract, post-vitrectomy cataract, radiation-induced cataract; and presbyobia, such as incipient presbyopia, functional presbyopia, absolute presbyopia, premature presbyopia or nocturnal presbyopia.

The methods of the invention can also be used to treat disease caused by an alphaA- or alphaB-crystallin mutation. The mutation in alphaA- or alphaB-crystallin can lead to hereditary cataract. Examples of alphaA-crystallin mutations include, but are not limited to, R54C, G98R, R21L, R116C, and W9X. Examples of alphaB-crystallin mutations include, but are not limited to, 150delA (αB184), D140N, P20S, D109H and R120G. In some instances, the alphaB-crystallin mutation is R120G or D109H.

In certain embodiments, the compounds and formulations disclosed herein are used to treat a subject with a vision disorder, such as cataract or presbyopia. In certain embodiments, the compounds and formulations disclosed herein may be used to treat cataract of a subject, such as nuclear cataract, cortical cataract, posterior capsular cataract, congenital cataract, secondary cataract, traumatic cataract, radiation cataract. In certain embodiments, a subject has one or more symptoms of cataract, such as clouded vision, blurred vision, dim vision, trouble seeing at night, sensitivity to light and glare, need for brighter light for reading and other activities, seeing “halos” around lights, frequent changes in eyeglasses or contact lens prescription, fading or yellowing of colors, and double vision in a single eye.

In certain embodiments, the compounds and formulations disclosed herein may be used to treat presbyopia of a subject, such as incipient presbyopia, functional presbyopia, absolute presbyopia, premature presbyopia or nocturnal presbyopia. In certain embodiments, the subject has one or more symptoms of presbyopia, such as decreased focusing ability for near objects, eyestrain, difficulty reading fine print, fatigue while reading or looking at an illuminated screen, difficulty seeing clearly up close, less contrast when reading print, need for brighter and more direct light for reading, needing to hold reading material further away in order to see it clearly, or headaches, especially headaches when using near vision. In some embodiments the subject does not have a cataract in an eye afflicted with presbyopia.

In certain embodiments, the subject has a vision disorder in one eye. In certain embodiments, the subject has a vision disorder in both eyes.

The subject of the disclosure can be any vertebrate animal. In some preferred embodiments the subject is a human. The subject may be of any age. In some embodiments the subject may be between 25 and 100 years of age, or between 40 and 100 years of age, or between 50 and 100 years of age. The subject may be over 1 year of age, over 2 years of age, over 5 years of age, over 10 years of age, over 18 years of age, over 20 years of age, over 25 years of age, over 30 years of age, over 35 years of age, over 40 years of age, over 45 years of age, over 50 years of age, over 60 years of age, over 70 years of age, over 80 years of age or over 90 years of age. The subject may be 25 years of age or older.

The methods provided herein can be used to treat a disease or a condition that would benefit from reducing the likelihood of or reversing the aggregation of alpha-crystallin by administering an effective amount of at least one of the compounds or formulations described herein. An effective amount can be an amount that reduces or inhibits the aggregation of alpha-crystallin. In certain embodiments, compounds and formulations of the disclosure reduce alpha-crystallin aggregation in an eye by about 1% to about 100%, about 1% to about 90%, about 1% to about 80%, about 1% to about 70%, about 10% to about 50%, about 20% to about 40%, about 50% to about 90% or between 60% to about 95% relative to a pre-treatment value for alpha-crystallin aggregation.

In certain embodiments, the compound or salt of the disclosure is a pharmacological chaperone that binds to the alpha-crystallin, such as the pharmacological chaperone can bind to a concave pocket near the antiparallel beta strand dimer interface site of alpha-crystallin. The concave pocket of alpha-crystallin can comprise serine 66, leucine 79, aspartate 80, valine 81, lysine 82, histidine 83, phenylalanine 84, valine 97, isoleucine 114, serine 115, arginine 116, aspartate 117, phenylalanine 118, histidine 119, arginine 120, lysine 121 and tyrosine 122 of αB-crystallin or αA-crystallin. In certain embodiments, the pharmacological chaperone can be, for example, a small molecule or a sterol or a sterol mimetic.

The compounds and formulations disclosed herein may be used to inhibit the aggregation of alpha-crystallin by at least about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% when compared to a pre-treatment level or a level observed in biologically matched control subject or specimen that was not administered said compounds. The compound and formulations disclosed herein may inhibit the aggregation of the alpha-crystallin by between 1% and 100%, between 5% and 90%, between 10% and 80%, between 20% and 50%, between 50% and 95%, between 60% and 99% or between 40% and 70% when compared to a pre-treatment level or a level observed in biologically matched control subject or specimen that was not administered said compounds.

In certain embodiments, the amyloid forming protein can be selected from a group consisting of Hsp27, αA-crystallin, αB-crystallin, βB2-crystallin βB1-crystallin, γD-crystallin, Hsp22, Hsp20, tau, Alphasynuclein, IAPP, beta-amyloid, PrP, Huntingtin, Calcitonin, Atrial natriuretic factor, Apolipoprotein AI, Serum amyloid A, Medin, Prolactin, Transthyretin, Lysozyme, Beta 2 microglobulin, Gelsolin, Keratoepithelin, Cystatin, Immunoglobulin light chain AL, myocilin, and SIBM.

Alpha-crystallin aggregation in the lens may be measured with, for example, in vivo dynamic light scattering, light scattering assays, electron microscopy, centrifugation protein solubility assays, filter trap protein solubility assays, thioflavin T-fluorescence assays, high performance liquid chromatography, gel-permeation chromatography, size exclusion chromatography, anti-amyloid antibody assays. In certain embodiments, exemplary methods of the disclosure for measuring alpha-crystallin aggregation in the lens are described in: K. Dierks et al, SPIE Vol. 2330 Lasers in Ophthalmology 11, 112-121 (1994); R. Ansari, Journal of Biomedical Optics January/February, Vol. 9, No. 1, 22-37 (2004); and X. Pei et al, Br J Ophthalmol 92, 1471-1475 (2008), the contents of each of which are incorporated by reference herein.

The compounds disclosed herein can inhibit cataract formation by at least about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% when compared to the level observed in biologically matched control subject or specimen that was not administered said compounds.

In certain embodiments, prior to treatment, the subject has experienced a loss of near vision. The subject may have experienced a loss of near vision which first occurred when the subject was 25 years of older. The subject may have been about 25 to 50 years old, such as about 25 to 40 years old, such as about 25 to 35 years old when the subject experienced a loss of near vision. The subject may have been diagnosed, e.g., diagnosed by a medical practitioner, as suffering a loss of near vision, or may self-identify as suffering a loss of near vision.

In certain embodiments, the subject has not yet experienced a decline in near vision. The subject may have one or more risk factors for the development of presbyopia. Risk factors include, but are not limited to: age over 40, hyperopia, an occupation with high near vision demands, gender, ocular disease or trauma, systemic disease, drug exposure (prescription and nonprescription), iatrogenic factors, proximity to the equator, exposure to high levels of UV radiation, poor nutrition, decompression sickness, and exposure to high ambient temperatures.

The subject may exhibit one or more symptoms of hyperopia. Some symptoms of hyperopia include: blurred vision, difficulty seeing objects up close, crossing of the eyes in children (esotropia). The subject may experience a loss of near vision. The loss of near vision may be a hyperopia or may not be a hyperopia. In some embodiments the loss of near vision is not related to a focus point of light rays behind the retina. In some embodiments the subject may not exhibit one or more symptoms of hyperopia.

A loss of near vision can be identified by an eye exam method, such as eye examinations used commonly in the field. A loss of near vision may be determined by assessing one or more of near vision acuity, habitual distance visual acuity, corrected near visual acuity, refractive error, optical power, Jaeger score, LogMAR score, ETDRS scale, reading speed, accommodative amplitude of the lens, or any other method known in the art. The subject may exhibit an age related loss of near vision as determined by one or more of the following of the methods above. Eye tests may be used to evaluate binocular vision, or used to evaluate each eye separately.

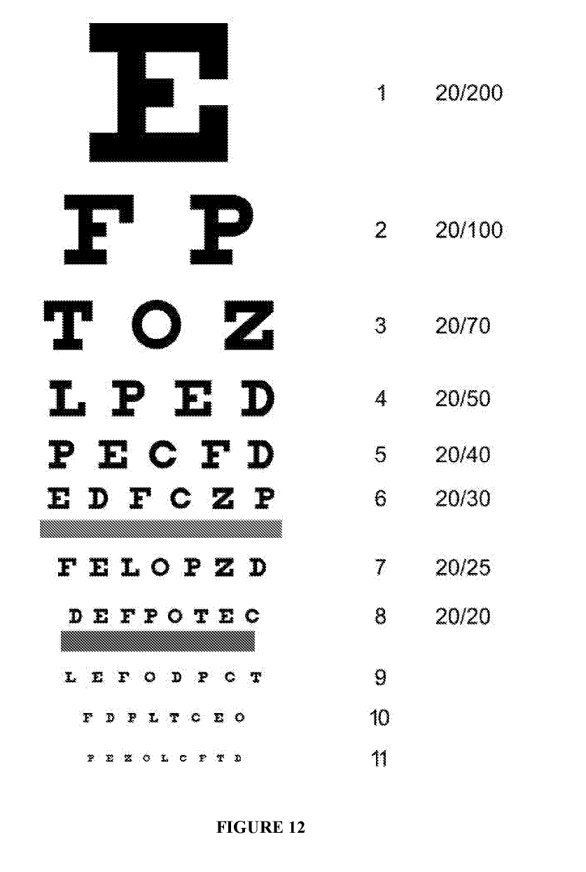

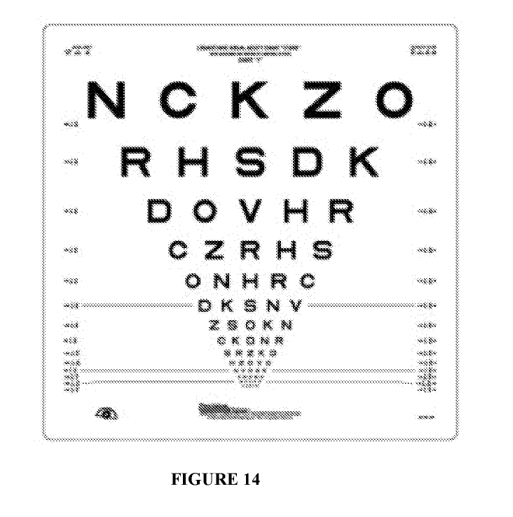

Vision acuity, or visual acuity, is commonly measured by requiring a subject to identify differently sized optotypes on a chart which is viewed at a set distance. The optotypes can be stylized letters or symbols. Viewing distance is typically such that the lens of the eye adjusts for either near vision or far distance vision. To measure near vision acuity the chart would be viewed at a set reading distance, typically 1. Many charts are known in the field, commonly used charts include the Snellen Chart, e.g., FIG. 12, the LogMar chart, e.g., FIG. 14, and the ETDRS chart. Some examples of visual acuity charts that can be used for illiterate subjects include, but are not limited to, the tumbling E chart, the Landolt C chart, and the LEA test. A reference value based on the size of optotypes that a person with ‘normal’ eyesight would be able to resolve is used to assign a visual acuity score. For example in a distance visual acuity test each line of optotypes is annotated with the distance from which a subject with ‘normal’ vision could read them. A subject then views the chart from 20 feet (or 6 meters) and reads the optotypes from largest to smallest, stopping at the smallest line which they are able to read with no mistakes, or with no more than one mistake, or with no more than two mistakes. If the smallest optotypes the subject can read are the ones annotated as 40 feet, then the subject has 20/40 vision, meaning that they can read at 20 feet what a subject with ‘normal’ eyesight can read at 40 feet. An efficient way to state acuity is by solving the fraction to a decimal number, thus a subject with 20/40 vision would have a decimal distance visual acuity of 0.5.

Near vision acuity can be measured in the same way but with a deceased viewing distance. In an example near vision test, the subject is instructed to cover one eye and use the other eye to view the eye chart of FIG. 12 from a distance of 16 inches. The tester determines the smallest size of character that the subject is able to read missing no more than one character, and correlates said character size with the distance from which a subject with ‘normal’ vision could read that size. The fraction may be converted to a decimal to give a decimal visual acuity value.

The subject of the methods of this disclosure may have a vision impairment determined by a near visual acuity of 0.9 or less. The subject of the methods of this disclosure may have a vision impairment determined by a near visual acuity of 0.8 or less. The subject of the methods of this disclosure may have a vision impairment determined by a near visual acuity of 0.6 or less. The subject of the methods of this disclosure may have a vision impairment determined by a near visual acuity of 0.4 or less.

Habitual distance visual acuity is the visual acuity of a subject with a correction. In some cases this is no correction, in some cases this may be eyeglasses or contact lenses. In cases where habitual distance visual acuity includes eye glasses or contact lenses the correction may not be optimal for the subjects' current needs.

In certain embodiments, the subject may experience difficulties with reading. The subject may have trouble reading small print, or reading print that they were previously able to read without trouble. The subject may have a decreased reading speed. The subject may complain of eye strain after extended periods of reading.

In certain embodiments, the subject has a near vision impairment that could alternatively be corrected with eye glasses or contact lenses having power of about +0.5 D or higher, about +1 D or higher or about +2D or higher. In some embodiments a subject of this disclosure could be identified as a person who occasionally or habitually uses eye glasses or contact lenses to correct a near vision impairment. The subject of this disclosure could be a person who occasionally or habitually uses reading glasses.

The near vision impairment may be determined by measuring the optical power of the lens of the eye. The optical power (also referred to as dioptric power, refractive power, focusing power, or convergence power) is the degree to which a lens converges light. The optical power is equal to the reciprocal of the focal length in meters and is expressed in diopters. For example a lens which can bring parallel rays of light to a focus at ⅓ of a meter has an optical power of 3 diopters. The ability to focus on near objects declines through life and levels off at 0.5 to 1 diopters at age 60.

An eye that has too much or too little optical power to focus light onto the retina may have a refractive error. A refractive error may be assessed using one or more of the following: a retinoscope, an automated refractor, a Shack-Hartmann wavefront sensor or a pinhole occluder.

The lens of a subject may have an optical power of less than 15 diopters before treatment with the compound of Formula (I). The lens of a subject may have an optical power of less than 20 diopters before treatment with the compound of Formula (I).

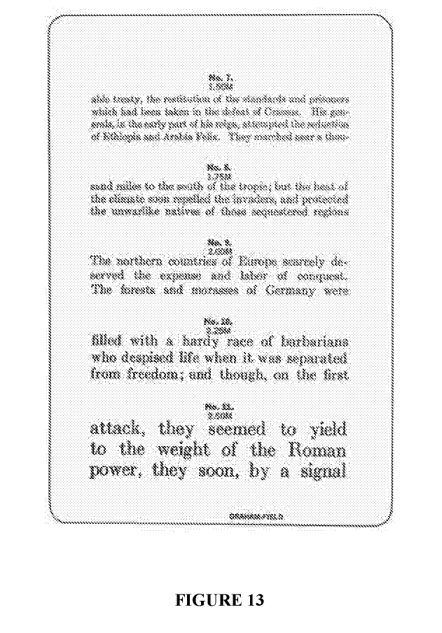

The near vision impairment may be determined by the Jaeger test scale, e.g., FIG. 13. The Jaeger chart is a type of eye chart used in testing near vision acuity. It is a card on which lines of paragraphs of text are printed at increasing size. Several variations of the Jaeger chart exist. This card is to be held by a subject at a fixed distance from the eye. The smallest print that the subject can read determines their visual acuity and their Jaeger score (J1 to J11 or larger). For example a subject who could read lines 4 and higher from the Jaeger chart would have a visual acuity of J4.

The near vision impairment may be determined by a score of J2 or higher on the Jaeger scale of the Jaeger test. The near vision impairment may be determined by a score of J3 or higher on the Jaeger scale of the Jaeger test. The near vision impairment may be determined by a score of J4 or higher on the Jaeger scale of the Jaeger test. The near vision impairment may be determined by a score of J5 or higher on the Jaeger scale. The near vision impairment may be determined by a score of J6 or higher on the Jaeger scale. The vision impairment may be determined by a score of J8 or higher on the Jaeger scale.

The near vision impairment may be determined by the LogMAR chart. When using the LogMAR chart, visual acuity is scored with reference to the Logarithm of the Minimum Angle of Resolution. A subject who can resolve details as small as 1 minute of visual angle scores LogMAR 0, (base-10 logarithm of 1 is 0); a subject who can resolve details as small as 2 minutes of visual angle (i.e., reduced acuity) scores LogMAR (base-10 logarithm of 2 is 0.3); and so on. A LogMAR score is calculated based on the number of letters the subject identifies correctly (each line is worth 0.1 LogMAR units).

The near vision impairment may be determined by a LogMAR score of 0.3 or higher. The near vision impairment may be determined by a LogMAR score of 0.4 or higher. The near vision impairment may be determined by a LogMAR score of 0.5 or higher. The near vision impairment may be determined by a LogMAR score of 0.6 or higher.

Use of the methods of this disclosure may treat or prevent presbyopia. Use of the methods of this disclosure by a subject who has not yet experienced symptoms of presbyopia may prevent or delay the onset of presbyopia.

Use of the methods of this disclosure by a subject with presbyopia may prevent or delay the progression of presbyopia. In some embodiments the subject does not experience a decline in near vision acuity over a period of time while being administered the compound of Formula (I).

Use of the methods of this disclosure by a subject with presbyopia may treat the presbyopia resulting in an improvement in near vision acuity. The improvement of the near vision of the subject may comprise an improvement in one or more of visual acuity, optical power, accommodative amplitude of the lens, Jaeger scale score, LogMAR scale score, ETDRS scale, reading speed, or refractive error.

In certain embodiments, the improvement of the near vision of the subject may comprise an improvement in near visual acuity relative to a pre-treatment near visual acuity value. In certain embodiments, the methods of the disclosure improve near vision impairment to a degree that is about equivalent to eye glasses or contact lenses having a power of about +0.5 D or higher, about +1 D or higher or about +2D or higher. In certain embodiments, the methods of the disclosure can replace treatment with eye glasses or contact lenses or surgical procedures.

In certain embodiments, methods of the disclosure result in improvement in near vision acuity of the subject. The methods of this disclosure may increase the optical power of a lens of a subject. In certain embodiments, the optical power of a lens may improve by at least 0.1 diopters relative to a pre-treatment optical power of the lens. In certain embodiments, the optical power of a lens may improve by at least 1 diopter relative a pre-treatment optical power of the lens. In certain embodiments, the optical power of a lens may improve by at least 5 diopters relative to a pre-treatment optical power of the lens. In certain embodiments, the optical power of a lens may improve by between 0.1 and 20 diopters relative to a pretreatment optical power of the lens. In certain embodiments, the optical power of a lens may improve by between 0.1 and 10 diopters relative to a pre-treatment optical power of the lens. In certain embodiments, the optical power of a lens may improve by between 1 and 10 diopters relative to a pre-treatment optical power of the lens. In certain embodiments, the optical power of a lens may improve by between 1 and 5 diopters relative to a pre-treatment optical power of the lens.

In certain embodiments, the treatment improves the subjects' near vision, e.g., by 1, 2, 3, 4, or 5 Jaeger lines, e.g., as measured by the Jaeger test scale, relative to a pre-treatment measurement. The treatment may improve a subjects' near vision by 1-7 Jaeger lines, or by 1-5 Jaeger lines, or by 1-3 Jaeger lines, relative to a pre-treatment measurement.

In certain embodiments, the treatment corrects the subjects' near vision, e.g., by 0.02, 0.04, 0.06, 0.1, 0.2, 0.3, 0.4, 0.5, or more than 0.5 LogMAR units. The treatment may correct subjects' near vision by 0.02-0.9 LogMAR units, or by 0.1-0.8 LogMAR units, or by 0.2-0.5 LogMAR units.

The McDonald-Shadduck scoring system can be used to determine the severity of various ocular symptoms upon administration of a compound of the present invention in rabbits. The scoring system can use a scale ranging from 0-6, where a higher number indicates greater severity of the ocular condition. The McDonald-Shadduck scoring system can be used to assess, for example, corneal opacity, corneal vascularization, conjunctival chemosis and swelling, conjunctival discharge, and corneal staining.

The McDonald-Shadduck scoring system can be used to grade conjunctival discharge. Conjunctival discharge can be used to describe discharge that is a whitish or gray precipitate. Discharge that is clear, inspissated, congealed, or mucoid found in the medial canthus of the rabbits is not scored as part of the conjunctival discharge scale.

Aqueous flare can be measured by the presence of the Tyndall phenomenon in the anterior chamber of the eye. The Tyndall phenomenon can be used to describe light scattering by particles in a colloid or particles in a fine suspension. The intensity of the Tyndall phenomenon can be scored by comparing the normal Tyndall effect observed when a slitlamp beam passes through the lens with the passage of a slitlamp beam passed through the anterior chamber. The presence of an aqueous flare can be indicative of a breakdown of the blood-aqueous barrier.

Iris involvement can be measured using the McDonald-Shadduck scoring system.

The primary, secondary, and tertiary vessels of the iris can be used an aid to determine a subjective ocular score for iris involvement. The intensity of iris involvement can increase when hyperaemia of the vessels is high, and there is greater involvement of the secondary and tertiary vessels.

### Combination Therapy

The compounds, or the pharmaceutically acceptable salts thereof, provided herein can be administered in combination with one or more therapeutic agents.

A compound or salt described herein, or a pharmaceutically acceptable salt thereof, may be co-administered with a second therapeutic agent, wherein the compound described herein, or a pharmaceutically acceptable salt thereof, and the second therapeutic agent modulate different aspects of the disease, disorder or condition being treated, thereby providing a greater overall benefit than administration of either therapeutic agent alone.

Different therapeutically-effective dosages of the compounds disclosed herein can be utilized in formulating a pharmaceutical formulation or in treatment regimens when the compounds disclosed herein are administered in combination with one or more additional agent. Therapeutically-effective dosages of drugs and other agents for use in combination treatment regimens is optionally determined by means similar to those set forth hereinabove for the therapeutic agents themselves. Furthermore, the methods of prevention/treatment described herein encompasses the use of metronomic dosing, for example, providing more frequent, lower doses in order to minimize toxic side effects. A combination treatment regimen can encompass treatment regimens in which administration of a compound described herein, or a pharmaceutically acceptable salt thereof, is initiated prior to, during, or after treatment with a second agent described herein, and continues until any time during treatment with the second agent or after termination of treatment with the second agent. The disclosure also includes treatments in which a compound described herein, or a pharmaceutically acceptable salt thereof, and the second agent being used in combination are administered simultaneously or at different times and/or at decreasing or increasing intervals during the treatment period. Combination treatment further includes periodic treatments that start and stop at various times to assist with the clinical management of the patient.

For combination therapies described herein, dosages of the co-administered compounds vary depending on the type of co-drug employed, on the specific drug employed, on the disease or condition being treated and so forth. In certain embodiments, when co-administered with one or more other therapeutic agents, the compound provided herein is administered either simultaneously with the one or more other therapeutic agents, or sequentially.

The compounds described herein or the pharmaceutically acceptable salts thereof, as well as combination therapies, may be administered before, during or after the occurrence of a disease or condition, and the timing of administering the composition containing a compound varies. The compounds described herein can be used as a prophylactic and may be administered continuously to subjects with a propensity to develop conditions or diseases in order to prevent the occurrence of the disease or condition. The compounds described herein and compositions thereof may be administered to a subject during or as soon as possible after the onset of the symptoms. A compound described herein may be administered as soon as is practicable after the onset of a disease or condition is detected or suspected, and for a length of time necessary for the treatment of the disease. The length required for treatment may vary, and the treatment length is adjusted to suit the specific needs of each subject. For example, a compound or salt described herein or a formulation containing the compound or salt can be administered for at least 2 weeks, about 1 month to about 5 years.

In certain embodiments, compounds or salts described herein can be used in combination with anti-apoptotic compounds. Anti-apoptotic compounds include, but are not limited to, lipoic acid, Humanin peptides, 3,6-dibromocarbazole piperazine derivatives of 2-propanol, Ku70 peptides, 4-phenylsulphanyl-phenylamine derivatives, IDN-6556, Anilinoquinazolines (AQZs), Nicotinyl aspartyl ketones, M826|M867, Pifithrin-α, INO-1001, FR255595, 3AB, NU1025, INH2BP, GP16150 and PJ34.

Compounds or salts of the disclosure may be used in combination with antioxidants. Antioxidants can include, for example, lipoic acid, glutathione, ascorbate, vitamin E, Uric acid, melatonin, vitamin C, Tirilazad, NXY-059, carotenes and ubiquinol. Reducing compounds can also be used to reduce disulfide bonds within proteins of the lens.

Additional therapeutic agents contemplated for combination therapy include, but are not limited to, analgesics, anesthetics, artificial tears, enzyme inhibitors, cytokine inhibitors, anti-inflammatory agents, antibiotic agents, antibacterial agents, antiviral agents, antifungal agents, antiprotozoal agents, or a combination thereof. The compounds described herein can also be used in combination with one or more ocular therapeutic agents. The compounds described herein can also be used in combination with an inhibitor of an enzyme that metabolizes and/or inactivates the compounds described herein. The compounds described herein can also be used in combination with an anti-inflammatory or cytokine inhibitor to reduce any safety issues or side effects stemming from possible inflammatory liabilities associated with the compounds disclosed herein. The compounds described herein can also be used in combination with other compounds known to have activity against cataracts. The compounds described herein can also be used with anti-apoptotic compounds or with antioxidants.

The compounds described herein can be used in combination with an analgesic or anesthetic agent. In certain embodiments, the analgesic or anesthetic agent comprises paracetamol, an opiate, diproqualone, phenazone, cocaine, or lidocaine. In certain embodiments, the opioid is a natural opium alkaloid, phenylpiperidine derivative, diphenylpropylamine derivative, benzomorphan derivative, oripavin derivative, or morphinan derivative. In some embodiments, the analgesic is a salicylic acid derivative, pyrazolone, or anilide. In other embodiments, the analgesic is an ergot alkaloid, corticosteroid derivative, or selective serotonin (5HT1) agonist. Examples of local anesthetics include, but are not limited to, Esters of aminobenzoic acid like metabutethamine, procaine, tetracaine, chloroprocaine, benzocaine; Amides like bupivacaine, lidocaine, mepivacaine, prilocaine, butanilicaine, cinchocaine, etidocaine, articaine, ropivacaine, levobupivacaine, tetracaine, chloroprocaine, benzocaine; Esters of benzoic acid like cocaine; Other local anesthetics like ethyl chloride, dyclonine, phenol, capsaicin.

The compounds described herein can be used in combination with an anti-inflammatory agent. The anti-inflammatory agent can be a non-steroidal anti-inflammatory agent. The anti-inflammatory agent can be a glucocorticosteroid. The non-steroidal anti-inflammatory agent can be a butylpyrazolidine, an acetic acid derivative, oxicam, propionic acid derivative, fenamate, or coxib. Examples of anti-inflammatory agents include, but are not limited to, Butylpyrazolidines like phenylbutazone, mofebutazone, oxyphenbutazone, clofezone, kebuzone; Acetic acid derivatives and related substances like indometacin, sulindac, tolmetin, zomepirac, diclofenac, alclofenac, bumadizone, etodolac, lonazolac, fentiazac, acemetacin, difenpiramide, oxametacin, proglumetacin, ketorolac, aceclofenac, bufexamac, indometacin combinations, diclofenac combinations; Oxicams like piroxicam, tenoxicam, droxicam, lornoxicam, meloxicam; Propionic acid derivatives like ibuprofen, naproxen, ketoprofen, fenoprofen, fenbufen, benoxaprofen, suprofen, pirprofen, flurbiprofen, indoprofen, tioprofenoic acid, oxaprozin, ibuproxam, dexibuprofen, flunoxaprofen, alminoprofen, dexketoprofen, naproxcinod; Fenamates like mefenamic acid, tolfenamic acid, flufenamic acid, meclofenamic acid; Coxibs like celecoxib, rofecoxib, valdecoxib, parecoxib, etoricoxib, lumiracoxib; Other antiinflammatory and antirheumatic agents like nabumetone, niflumic acid, azapropazone, glucosamine, benzydamine, glucosaminoglycan polysulfate, proquazone, orgotein, nimesulide, feprazone, diacerein, morniflumate, tenidap, oxaceprol, chondroitin sulfate; Corticosteroids like the Mineralocorticoids aldosterone, fludrocortisones, desoxycortone, and the Glucocorticoids betamethasone, dexamethasone, fluocortolone, methylprednisolone, paramethasone, prednisolone, prednisone, triamcinolone, hydrocortisone, cortisone, prednylidene, rimexolone, deflazacort, cloprednol, meprednisone, cortivazol.

The compounds described herein can be used in combination with an antibiotic agent. The antibiotic agent can be an aminoglycoside, ansamycin, carbacephem, carbapenem, cephalosporin, glycopeptide, lincosamide, lipopeptide, macrolide, monobactam, nitrofurans, penicillin, polypeptide, quinolone, sulfonamide, tetracycline amikacin, gentamicin, kanamycin, neomycin, netilmicin, tobramicin, paromomycin, geldanamycin, herbimycin, loracarbef, ertapenem, doripenem, imipenem, meropenem, cefadroxil, cefazolin, cefalotin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftobiprole, teicoplanin, vancomycin, telavancin, clindamycin, lincomycin, daptomycin, azithromycin, clarithromycin, dirithromycin, erythromycin, roxithromycin, troleandomycin; telithreomycin, spectinomycin, aztreonam, furazolidone, nitrofurantoin, amoxicillin, ampicillin, azlocillin, carbinicillin, cloxacillin, dicloxacillin, flucloxacillin, mezlocillin, methicillin, nafcillin, oxacillin, penicillin G, penicillin V, piperacillin, temocillin, ticarcillin, bacitracin, colistin, polymyxin B; Quinolone derivatives like ciprofloxacin, enoxacin, gatifloxacin, levofloxacin, lomefloxacin, moxifloxacin, nalidixic acid, norfloxacin, ofloxacin, trovafloxacin, grepafloxacin, sparfloxacin, temafloxacin, danofloxacin, difloxacin, enrofloxacin, ibafloxacin, marbofloxacin, orbifloxacin, sarafloxacin, mafenide, sulfonamidochrysoidine, sulfacetamide, sulfadiazine, silver sulfadiazine, sulfamethoxazole, sulfanilimide, sulfasalazine, sulfisoxazole, trimethoprim, trimethoprim/sulfamethoxazole, demeclocycline, doxycycline, minocycline, oxytetracycline, tetracycline, clofazimine, dapsone, capreomycin, cycloserine, ethambutol, ethioamide, isoniazid, pyrazinamide, rifampin, refampicin, rifabutin, rifapentine, streptomycin; or other antibiotic agents like arsphenamine, chloramphenicol, fosfomycin, fusidic acid, linezolid, metronidazole, mupirocin, platensimycin, quinupristin/dalfopristin, rifaximin, thiampheniol, tigecycline, tinidazole.

The compounds described herein can be used in combination with an antibacterial agent. The antibacterical agent can be, for example, an alcohol, an aldehyde, a halogen-releasing compound, a peroxide, an anilide, a biguanide, a bisphenol, a halophenol, a heavy metal, a phenol, a cresol, a quaternary ammonium compound, ethanol, isopropyl alcohol, glutaraldehyde, formaldehyde, halogen releasing compounds, hydrogen peroxide, ozone, peracetic acid, biguanides, chlorhexidine, alexidine, polymeric biguanides, bisphenols, triclosan, hexachlorophene, silver compounds, mercury compounds, quaternary ammonium compounds, benzalkonium chloride, cetrimide, methylbenzethonium chloride, benzethonium chloride, cetaalkonium chloride, cetylpyridinium chloride, and dofanium chloride.

The compounds described herein can be used in combination with an antiviral agent. The antiviral agent can be a nucleoside reverse transcriptase inhibitor, a non-nucleoside reverse transcriptase inhibitor, a fusion inhibitor, an integrase inhibitor, a nucleoside analog, a protease inhibitor, or a reverse transcriptase inhibitor. Examples of antiviral agents include, but are not limited to, abacavir, aciclovir, acyclovir, adefovir, amantadine, amprenavir, ampligen, arbidol, atazanavir, boceprevir, cidofovir, darunavir, delavirdine, didanosine, docosanol, edoxudine, efavirenz, emtricitabine, enfuvirtide, entecavir, famciclovir, fomivirsen, fosamprenavir, foscarnet, fosfonet, ganciclovir, ibacitabine, imunovir, idoxuridine, imiquimod, indinavir, inosine, interferon type III, interferon type II, interferon type I, interferon, lamivudine, lopinavir, loviride, maraviroc, moroxydine, methisazone, nelfinavir, nevirapine, nexavir, oseltamivir, peginterferon alfa-2a, penciclovir, peramivir, pleconaril, podophyllotoxin, raltegravir, ribavirin, rimantadine, ritonavir, pyramidine, saquinavir, stavudine, tea tree oil, tenofovir, tenofovir disoproxil, tipranavir, trifluridine, trizivir, tromantadine, truvada, valaciclovir (Valtrex), valganciclovir, vicriviroc, vidarabine, viramidine, zalcitabine, zanamivir, zidovudine.

The compounds described herein can be used in combination with an antifungal agent. The antifungal agent can be a polyene antifungal, an imidazole, triazole, or thiazole antifungal, a triazole antifungal, a thiazole antifungal, an allylamine derivative, or an echinocandin derivative. Examples of antifungal agents include, but are not limited to, Polyene derivatives like natamycin, rimocidin, filipin, nystatin, amphotericin B, candicin, hamycin; Imidazole derivatives like miconazole, ketoconazole, clotrimazole, econazole, omoconazole, bifonazole, butoconazole, fenticonazole, isoconazole, oxiconazole, sertaconazole, sulconazole, tioconazole; Tetrazole derivatives like fluconazole, itraconazole, isavuconazole, posaconazole, voriconzaole, terconazole, albaconazole; Thiazole derivatives like abafungin; Allylamine derivative like terbifine, naftifine, butenafine; Echinocandin derivatives like anidulafungin, caspofungin, micafungin; Other antifungals like polygodial, benzoic acid, ciclopirox, tonaftate, undecylenic acid, flycytosine, griseofulvin, haloprogin, sodium bicarbonate, pirctone olamine, zinc pyrithione, selenium sulfide, tar, or tea tree oil.

The compounds described herein can be used in combination with an antiprotozoal agent. Examples of antiprotozoals include, example, eflornithine, furazolidone, melarsoprol, metronidazole, ornidazole, paromomycin sulfate, pentamidine, pyrimethamine, tinidazole, nifuratel, Doxycycline, proguanil with atovaquone, chloroquine, and mefloquine.

The compounds described herein can be used in combination with an ocular therapeutic agent. Ocular therapeutic agents include therapeutic agents that are used to treat ophthalmic related diseases or conditions. Such ocular therapeutic agents include, for example, immunomodulators, corticosteroids, beta-blockers, alpha agonists, carbonic anhydrase inhibitors, antibiotics, antihistimines, and protasglandins.

### EXAMPLES

### Example 1: Illustrative Examples of Formulations

Illustrative formulations that can be used in a method of the invention are shown in TABLE 2 below:

TABLE 2

| | Concentration | | |
|---|---|---|---|
| | of compound | | |
| Dose form | (C29) | Excipients | Form |
| Aqueous | 0.04% (1 mM) | 2-hydroxypropyl-β- | Solution |
| solution | | cyclodextrin | |
| Ointment | 0.4% and 4% | Petrolatum, | Anhydrous, |
| | | lanolin, | dissolved in |
| | | mineral oil | oleaginous |
| | | | phase |
| Aqueous | 0.3% and 3% | Gellan gum, | ~500 nM |
| suspension | | tromethamine, | particle |
| | | mannitol, | suspension |
| | | boric acid, | |
| | | polysorbate-80, | |
| | | benzalkonium | |
| | | chloride | |
| Oil in water | 0.06% and 0.6% | isopropyl palmitate, | solution in |
| nanoemulsion | | polysorbate-80 | oil phase; ~50 |
| | | | nM droplets |

### Example 2: Pharmacokinetic Studies

To test the efficacy of a compound of the invention, a bioanalytical method was employed to allow for pharmacokinetic studies. The bioanalytical method was sensitive at a scale of about 15 nM of the compound in plasma, and about 20 nM of the compound in ocular tissues. New Zealand white rabbits were given six doses over three days for each topical arm, and a single injection for intravitreal and intracameral injections, with time points at two hours and 24 hours post-injection.

### Lens Exposure of the Topical Agents

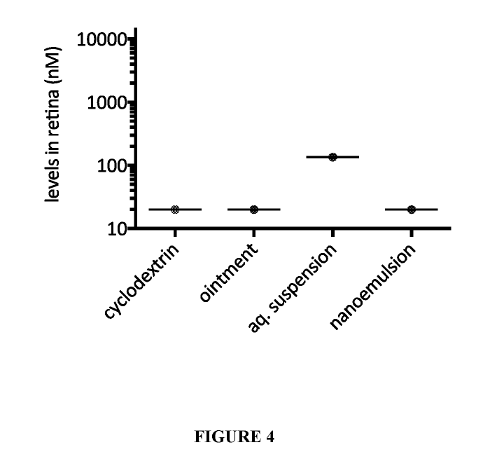

The exposure of a compound of the invention (C29) was measured in the lens of the rabbits, which can display slow diffusion because the lens is a protein-rich, dense tissue area in the anterior of the eye. Ciliary process levels were used as a measure of exposure of the compound in the lens. TABLE 3 below and FIG. 4 display the results of the experiments.

TABLE 3

| | | Ciliary process | |
|---|---|---|---|
| Formulation | Dose | levels (nM) | |
| Cyclodextrin | b.i.d. for 14 days | <20 | nM |
| Ointment | b.i.d. for 3 days | <20 | nM |
| Aqueous Suspension | b.i.d. for 3 days | one lens w/135 nM, | |
| | | others <20 nM | |
| Nanoemulsion | b.i.d. for 3 days | <20 | nM |

### Corneal Exposure of the Topical Agents

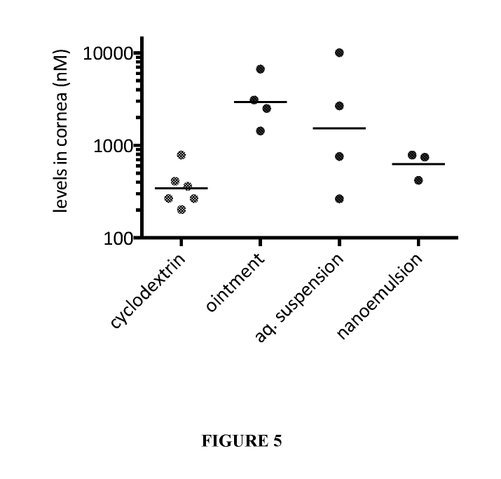

The exposure of a compound of the invention (C29) was measured in the cornea of the rabbits. To get to the cornea, the compound must pass through the cornea, or sclera, to access the internal structures of the eye. TABLE 4 below and FIG. 5 display the results of the experiments.

TABLE 4

| Formulation | Dose | Corneal levels (nM) | |
|---|---|---|---|
| Cyclodextrin | b.i.d. for 14 days | 200-800 | nM |
| Ointment | b.i.d. for 3 days | 1,440-6,710 | nM |
| Aqueous Suspension | b.i.d. for 3 days | 270-10,100 | nM |

| | | |
|---|---|---|
| Nanoemulsion | b.i.d. for 3 days | 420-790 |

### Retinal Exposure of the Topical Agents

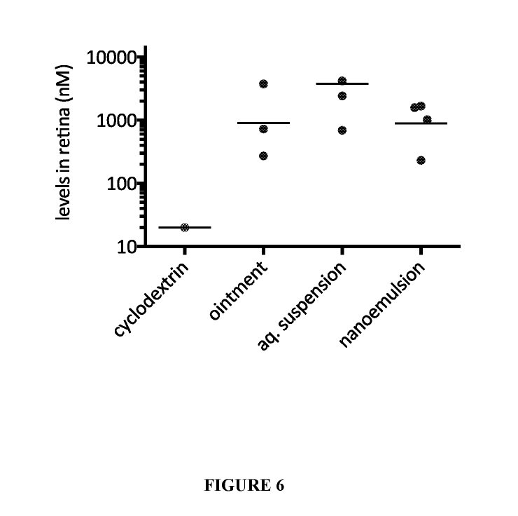

The exposure of a compound of the invention (C29) was measured in the retina of the rabbits, which can display fast diffusion because the retina is a lipid-rich, soft tissue area in the back of the eye. TABLE 5 below and FIG. 6 display the results of the experiments.

TABLE 5

| Formulation | Dose | Retinal Levels (nM) | |
|---|---|---|---|
| Cyclodextrin | b.i.d. for 14 days | <20 | nM |
| Ointment | b.i.d. for 3 days | 272-3,770 | nM |
| Aqueous Suspension | b.i.d. for 3 days | 690-28,900 | nM |
| Nanoemulsion | b.i.d. for 3 days | 231-1,680 | nM |

### Ciliary Body Exposure of the Topical Agents

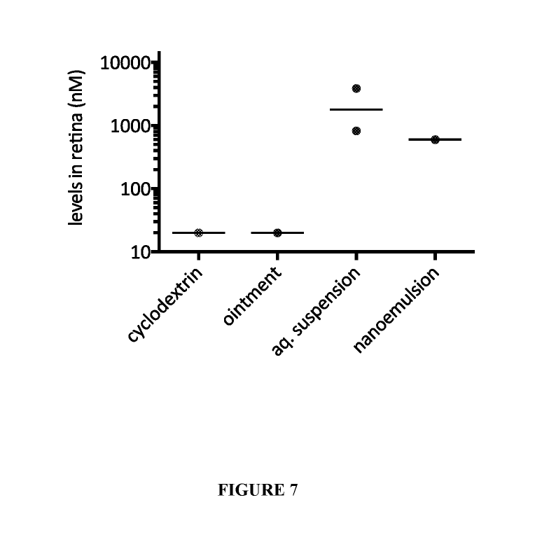

The exposure of a compound of the invention (C29) was measured in the ciliary bodys of the rabbits, which can display fast diffusion because the ciliary body is a lipid-rich, soft tissue in the anterior of the eye. TABLE 6 below and FIG. 7 display the results of the experiments.

TABLE 6

| | | Ciliary Process | |
|---|---|---|---|
| Formulation | Dose | Levels (nM) | |
| Cyclodextrin | b.i.d. for 14 days | <20 | nM |
| Ointment | b.i.d. for 3 days | <20 | nM |
| Aqueous Suspension | b.i.d. for 3 days | 830 nM and 3,870 nM | |
| Nanoemulsion | b.i.d. for 3 days | 600 | nM |

### Ex Vivo Human Tissue Pharmacokinetic Experiments

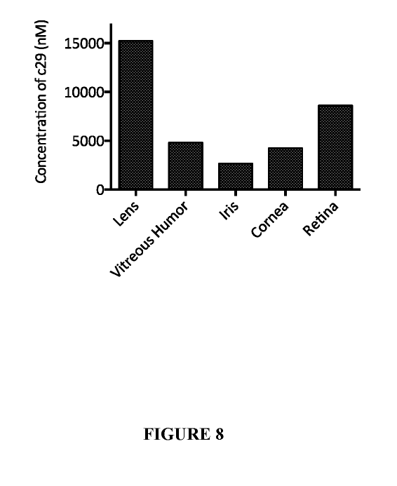

Pharmacokinetic experiments were performed on entire human globes (eye without appendages) to determine exposure of the compound (C29) in various areas of the eye. Each globe was obtained less than 24 hours after collection and then soaked in either a solution of 1 mM API in 8% cyclodextrin in PBS or only PBS for six days at room temperature. The results are displayed below in table TABLE 7 and FIG. 8.

TABLE 7

| | Calculated | Dilution Corrected |
|---|---|---|
| Sample Name | Concentration (nM) | Concentration (nM) |
| Human Lens PBS | <150 | |
| Human Lens C29 | 7620 | 15240 |
| Human Vitreous Humor PBS | <150 | |
| Human Vitreous Humor C29 | 2410 | 4820 |
| Human Iris PBS | <150 | |
| Human Iris C29 | 1330 | 2660 |
| Human Cornea PBS | <150 | |
| Human Cornea C29 | 2120 | 4240 |
| Human Retina PBS | <150 | |
| Human Retina C29 | 4310 | 8620 |

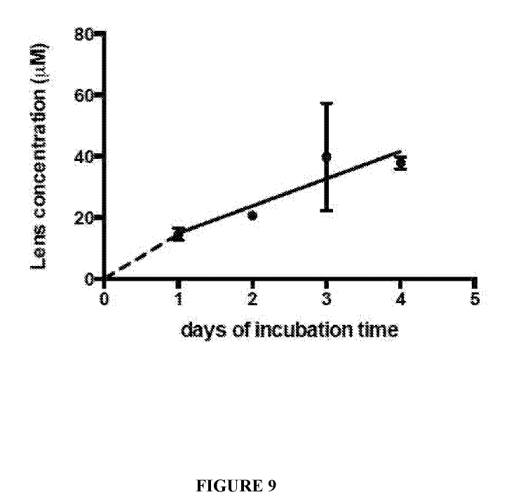

In a similar experiment, entire human globes were used to determine the kinetics of compound accumulation in the lens. A total of eight globes were soaked in a solution of 1 mM API in 8% cyclodextrin in PBS for one, two, three, or four days at room temperature. The results are displayed below in TABLE 8 and FIG. 9.

TABLE 8

| | Average | |
|---|---|---|
| Days of | Concentration | Standard |
| Incubation | in Lens (μM) | Deviation |
| 1 | 14.5 | 1.9 |
| 2 | 20.7 | 0.7 |
| 3 | 39.8 | 17.5 |
| 4 | 37.8 | 2.0 |

### Example 3: Comparative Formulation Study in Rabbits

New Zealand white albino rabbits were used to test different formulations and routes of administration of a compound of the invention. The rabbits were given six doses over three days for each topical arm, and a single injection for intravitreal and intracameral injections, with time points at two hours and 24 hours post-injection.

The control formulation tested was an aqueous solution containing 0.04% of the compound in 8% 2-hydroxypropyl-β-cyclodextrin.

The ointment formulation contained 4.3% lanolin, 9.9% light mineral oil, 85.9% white petrolatum, and either 0.4% or 4% of the compound. In this formulation, the addition of the mineral oil was able to decrease the melting point and improve the fluidity of the ointment for easy expulsion from the storage tube. The lanolin was used to solubilize the compound.

The aqueous suspension was developed as a gellan gum-based suspension of about 500 nm-sized particles, 0.6% gellan gum (low acyl), 1.5-3% mannitol to bring the osmolarity to 293 mOsm, 0.6% tromethamine, 0.1% polysorbate-80, 0.01% benzalkonium chloride, 0.3% or 3% of the compound, and 0.53% boric acid to adjust the pH to 7.4. The gellan gum was added to enhance the viscosity and to allow for suspension of the solution. The tromethamine was added as a buffer. The polysorbate was added to aid dispersion of the compound. The benzalkonium chloride was added as a preservative. The 500 nm particles were created via microfluidization prior to addition of the gellan gum.

The nanoemulsion was a surfactant-stabilized oil-in-water emulsion, which contained 5% isopropyl palmitate, 32.5% PEG-400, 15% polysorbate-80, 0.01% benzalkonium chloride, and 0.06% or 0.6% of the compound. The formulation was microfluidized to create 50 nm droplets. The benzalkonium chloride was added as a preservative.

The topical aqueous suspension was also used for injection and was developed as a gellan gum-based suspension of about 500 nm-sized particles, 0.6% gellan gum (low acyl), 1.5-3% mannitol to bring the osmolarity to 293 mOsm, 0.6% tromethamine, 0.1% polysorbate-80, 1% or 3% of the compound, and 0.53% boric acid to adjust the pH to 7.4. The gellan gum was added to enhance the viscosity and to allow for suspension of the solution. The tromethamine was added as a buffer. The polysorbate was added to aid dispersion of the compound. The 500 nm particles were created via microfluidization prior to addition of the gellan gum. The suspension was injected into the vitreous humor (intravitreal injection) in a 25 μL dose or the aqueous humor (intracameral injection) as a 5 μL dose.

The rabbits were scored according to the McDonald-Shadduck scoring system as described above to determine the safety of the compound. The parenthetical numbers below reflect the scores from the McDonald-Shadduck scoring system.

In the rabbit that received the ointment formulation with 0.4% API, slight panus (1) was observed in both eyes on day zero, which was the day of dosing. Slight congestion (1) and swelling (1) was observed one hour after dosing in both eyes. All other scores were zero. By days 2 and 3, the scores for all symptoms were zero.

In the rabbits that received an intravitreal formulation with 1% or 3% API, slight congestion (1) and swelling (1) was observed two hours after dosing in both eyes of the animals. In the rabbit that received an intracameral formulation with 1% API, slight panus (1) was observed two hours after dosing in the left eye only. In the rabbit that received an intracameral formulation with 3% API, slight iris involvement (1) was observed two hours after dosing in the right eye only.

### Example 4: Evaluating the Ocular Distribution of Compound IIIC after Intravitreal Administration into New Zealand White Rabbits

### Animals

New Zealand White rabbits obtained from the Western Oregon Rabbit Company were used for this study. 12 animals were used in this study, and all animals used were male. Prior to treatment initiation, selection of animals for the study was based on a visual appraisal of good clinical condition and body weight specifications. Animals selected for use in this study were as uniform in age and weight as possible. Animal's weights ranged from about 2.58 to about 3.22 kilograms at the start of the experiment. All animals were healthy at the time of animal selection. All animals were identified by ear tag and by cage cards listing the animal identification number, study number, group, and sex of the animal.

The animals were housed in individual cages within the same room during the study. Primary enclosures were as specified in the USDA Animal Welfare Act (9 CFR, Parts 1, 2, and 3) and as described in the Guide for Care and Use of Laboratory Animals (National Research Council of the Academies, 2011, National Academy Press).

No other species were housed in the same room. The room was well ventilated (greater than 10 air changes per hour) with at least 60% fresh air. A 12-hour light/12-hour dark photoperiod was maintained, except when rooms were illuminated during the dark cycle to accommodate necessary study procedures. Room temperatures were maintained as per ASI SOPs.

Animals had ad libitum access to species specific chow. No contaminants were known to be present in the diet at levels that would interfere with the results of this study. Chlorinated, municipal tap water was made available ad libitum to each animal via water bottles. No contaminants were known to be present in the water at levels that would interfere with the results of this study. Records of annual water quality testing are maintained in the ASI archives. All study animals were acclimated to their designated housing for 8 to 16 days prior to the first day of dosing.

Prior to placement on study, each animal underwent an ophthalmic examination (slit-lamp biomicroscopy and indirect ophthalmoscopy). Ocular findings were scored according to a modified McDonald-Shadduck Scoring System and were recorded on a standardized data sheet. The acceptance criteria for placement on study were scores of “0” for all variables. Animals were assigned to one of two experimental groups based on body weight.

### Formulation

The compound of formula IIIC was tested at two different concentrations, 3% weight/volume and 0.5% weight/volume. For both concentrations the compound was formulated as an aqueous suspension. Details of the formulations are as follows: Formulation 1: 3% of ˜500 nm nanoparticles of a compound of formula IIIC in 0.6% tromethamine as buffer, 0.1% polysorbate-80 to aide in dispersion of the API, pH adjusted with boric acid to 7.4, and osmolarity adjusted with mannitol to 293 mOsm. Formulation 2: 0.5% of ˜500 nm nanoparticles of a compound of formula IIIC in 0.6% tromethamine as buffer, 0.1% polysorbate-80 to aide in dispersion of the API, pH adjusted with boric acid to 7.4, and osmolarity adjusted with mannitol to 293 mOsm.

To prepare Formulations 1 and 2, 500 mL of buffer was prepared with mannitol, tromethamine, boric acid, polysorbate-80 (at quantities indicated above) and double deionized water to 500 mL. The buffer was mixed and then filtered through a sterile 0.22 m filter. For a 3% suspension of the compound of formula IIIC, 150 mg of compound was added to 5 mL of the buffer. A microfluidics LV-1 low volume microfluidizer was then used to reduce particle size. The microfluidizer had a chamber capacity of 6 mL. The microfluidizer was flushed with double deionized water five times, then sterile buffer two times. Using an air-filled syringe, the liquid was pushed out of the chamber until air bubbles are visible and liquid stops exiting the chamber. Using a 10 mL sterile syringe and sterile large gauge needle, the formulation was drawn up and processed with the microfluidizer at 30,000 psi. This was repeated for four passes, fully ejecting the receiving syringe as the input syringe for subsequent passes, and using a new sterile receiving syringe each pass to avoid cross-contamination. After five passes, syringeability was confirmed with a 31G needle. The particle size was then characterized by dynamic light scattering.

To dilute a 3 wt % mixture of the compound of formula IIIC to a 0.5 wt % mixture, sterile API buffer plus a mannitol solution was added. A stock solution of 13.52 mg/mL mannitol was prepared by dissolving 135.2 mg/10 mL API buffer. The stock solution of mannitol was filtered through a sterile filter. The mannitol stock solution was used to dilute the 3 wt % drug mixture to a 0.5 wt % drug mixture by taking 500 μL of 3 wt % drug suspension and adding 2.5 mL of mannitol stock solution.

The formulations were refrigerated at 4° C. prior to use. Prior to administration the formulations were warmed to room temperature. Each vial was vigorously swirled or vortexed to ensure the suspensions were homogenous. There were no noted color changes or signs of microbial growth.

### Dosing

Animals were assigned to one of two experimental groups based on body weight, such that each group contained 6 animals. Animals in group 1 were administered 25 μL for formulation 1 by intravitreous injection, while animals in group 2 were administered 25 μL of formulation 2. Within each group animals were further divided into 3 time points, 2 hours, 1 day and 7 days. Each time point consisted of two animals. Administration of formulations 1 and 2 occurred at time 0.

For intravitreal injections animals were anesthetized with an intramuscular injection of ketamine hydrochloride (12 to 20 mg/kg) and xylazine (5 mg/kg). One to two drops of topical proparacaine hydrochloride anesthetic (0.5%) were applied to the animal's eyes prior to the surgical procedure. The eyes were cleaned with Betadine and then rinsed with balanced salt solution (BSS). Test article was drawn up directly into a 0.3 mL insulin syringe with a 31G 5/16 inch needle, and injections were made 4 to 5 mm away from the limbus. Once the needle was inserted, 25 μL of the test article was injected. The needle was removed and the eye rinsed with BSS. Triple antibiotic ophthalmic ointment was administered in both eyes of each animal following the injection procedure. Animals were monitored during recovery.

General health observations were recorded daily starting on Day 0 and continued throughout the course of the study. Gross ocular examinations, which consisted of a visual appraisal of swelling, discharge, and irritation to the eye, were taken daily starting on Day 0 and continued throughout the course of the study.

### Sample Collection

Animals were euthanized at the designated time points by an intravenous injection of pentobarbital (150 mg/kg). The euthanasia procedure was performed in compliance with the 2013 American Veterinary Medical Association (AVMA) Guidelines on Euthanasia. Immediately following euthanasia, both eyes from each animal were enucleated, dissected, and the ocular tissues were collected. Samples from each eye in each animal remained separate and were not pooled. Samples were flash frozen in liquid nitrogen and stored at −60 to −80° C. until LC-MS/MS analysis. The concentration of compound IIIC in each collected sample was measured by LC-MS/MS. Standards were prepared in blank homogenized New Zealand white rabbit ocular tissues, vitreous humor, or plasma. Working solutions were prepared in 50:50 acetonitrile: water. Working solutions were then added to the appropriate matrix to make calibration standards. Standards were treated identically to the study samples. Tissue and humor samples were manually extracted via precipitation with acetonitrile.

### HPLC Conditions

Instrument: Waters Acquity UPLC; Column: Waters BEH phenyl, 30×2.1 mm id, 1.7 μm; Aqueous Reservoir (A): 0.1% formic acid in water; Organic Reservoir 0.1% formic acid in acetonitrile; Gradient Program:

| Time (min) | Grad. Curve | % A | % B |
|---|---|---|---|
| 0.0 | 6 | 60 | 40 |
| 2.25 | 6 | 0 | 100 |
| 2.4 | 6 | 60 | 40 |
| 3 | 6 | 60 | 40 |

HPLC Flow rate: 800 μL/min; Injection volume: 10 μL; Column temperature: 40° C.; Sample temperature: 8° C.; Strong autosampler wash:1:1:1 (v:v:v) water:methanol:isopropanol with 0.2% formic acid; Weak autosampler wash:4 mM ammonium formate

### Mass Spectrometer Conditions

Instrument: Waters Xevo TQ-S; Interface: Electrospray; Mode: Multiple reactions monitoring; Nebulizer gas: 7 bar; Desolvation gas: 1000 L/hr; Cone gas:150 L/hr; Collision gas: 0.15 mL/min; Desolvation temp: 450° C.; Capillary voltage: 3 kV

### Results

The test formulations (3 wt % or 0.5 wt %) were successfully administered via bilateral IVT injections into the eyes of 12 New Zealand White rabbits. There were no complications noted during the dosing events. All animals which were survived until Day 7 gained weight over the course of the study. All animals exhibited normal behavior and health during the study. There were no gross ocular observations of irritation, swelling, or discharge noted in any of the animals during the study.

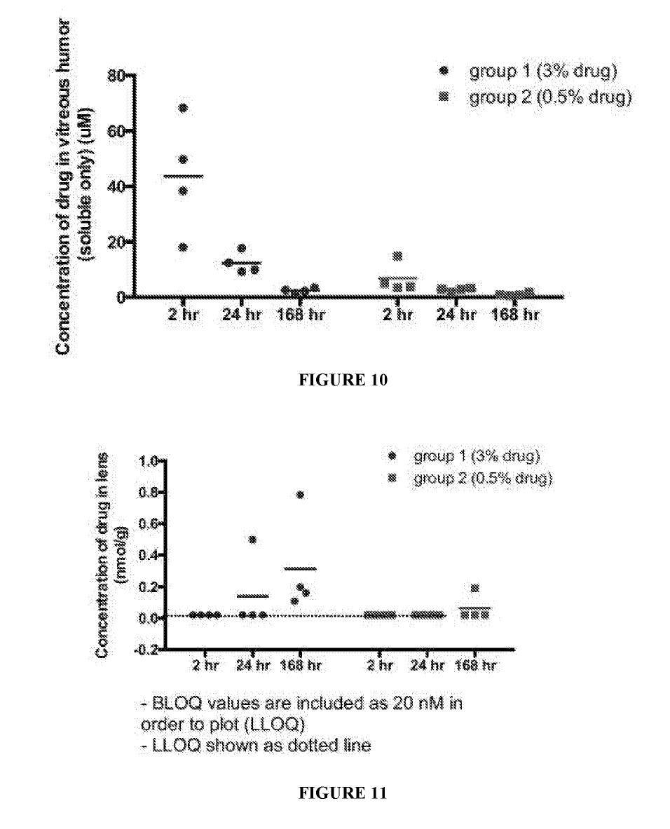

The results are displayed in FIG. 10 and FIG. 11. FIG. 10 depicts the concentration of 25-hydroxycholesterol in the vitreous humor at 3 wt % and 0.5 wt % following intravitreal administration in rabbits, i.e., 2 hr, 24 hr, and 168 hr following administration. The levels of the compound of formula IIIC in the vitreous humor were dose-dependent, with proportionally higher levels in the group treated with the 3% formulation than that treated with 0.5% formulation, and decreased steadily at later time points. FIG. 11 depicts the concentration of 25-hydroxycholesterol in the lens at 3 wt % and 0.5 wt % following intravitreal administration in rabbits, i.e., 2 hr, 24 hr, and 168 hr following administration. Levels in the lens were undetectable at earlier time points, and appeared to increase at later time points, with only the higher-dosed group at the last time point (7 days after dosing) consistently exhibiting detectable levels of the compound of formula IIIC, suggesting a delayed distribution to this tissue after IVT injection.

### Example 5: Treatment of Presbyopia with a Compound IIIC

A group of 5 patients with presbyopia are identified based on impaired near visual acuity in the LogMAR test. The patients are treated weekly with a composition of a compound of formula IIIC. Every month each patients' near visual acuity is measured on the LogMAR test. The patients' near visual acuity following treatment is compared to their pretreatment visual acuity.

### Example 6: Treatment of Loss of Near Vision Acuity with Compound IIIC

A group of 5 patients over the age of 40 years and without clinical signs of presbyopia are identified based on performance in the LogMAR test. The patients are treated with daily eyedrops containing composition of a compound of formula IIIC. Every 6 months patients' near visual acuity is measured on the LogMAR test. The patients' near visual acuity is compared to their pretreatment visual acuity.

### EMBODIMENTS

In certain aspects, the disclosure provides one or more of the following embodiments:

1. A method of treating or preventing a near vision impairment of a subject, comprising administering to a subject in need thereof a compound of Formula (III):

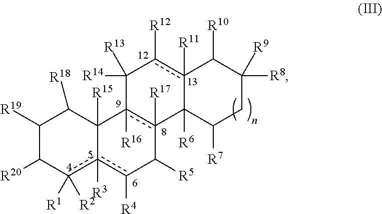

or a salt thereof, wherein:

R¹, R², R³, R⁴, R⁶, R⁸, R⁹, R¹¹, R¹², R¹³, R¹⁴, R¹⁵, R¹⁶, and R¹⁷ are independently selected from hydrogen, halogen, —OR³⁰, —SR³⁰, —OSO₃R³⁰, —OPO₃R³⁰, —N(R³¹)₂, —C(O)R³⁰, —C(O)OR³⁰, —OC(O)R³⁰, —NO₂, —CN, optionally substituted C₁-C₁₀ alkyl, optionally substituted C₂-C₁₀ alkenyl, optionally substituted C₂-C₁₀ alkynyl, optionally substituted carbocycle and optionally substituted heterocycle; R¹ taken together with R² is further selected from ═O, ═S, and ═N(R³¹); R⁸ taken together with R⁹ is further selected from ═O, ═S, and ═N(R³¹); R¹³ taken together with R¹⁴ is further selected from ═O, ═S, and ═N(R³¹); R⁹ and R¹⁰ taken together with the atoms to which they are attached may further form an optionally substituted carbocycle or optionally substituted heterocycle; and wherein R³ is absent when there is a double bond between carbons 5 and 6, R¹⁶ and R¹⁷ are absent when there is a double bond between carbons 8 and 9, R¹¹ is absent when there is a double bond between carbons 12 and 13; and R² and R³ are absent and there is a single bond between carbons 5 and 6 when there is a double bond between carbons 4 and 5;

R⁵, R⁷, R¹⁰, R¹⁸, R¹⁹ and R²⁰ are independently selected from hydrogen, halogen, —OR³⁰, —SR³⁰, —OSO₃R³⁰, —OPO₃R³⁰, N(R³¹)₂, —C(O)R³⁰, —C(O)OR³⁰, —OC(O)R³⁰, —NO₂, —CN, ═O, ═S, ═N(R³¹), optionally substituted C₁-C₁₀ alkyl, optionally substituted C₂-C₁₀ alkenyl, C₂-C₁₀ alkynyl, optionally substituted carbocycle and optionally substituted heterocycle; each R³¹ is independently selected from hydrogen, —OR³⁰, —SR³⁰, —S(O)R³⁰, —S(O)₂R³⁰, C(O)R³⁰, —C(O)OR³⁰, optionally substituted C₁-C₁₀ alkyl, optionally substituted C₂-C₁₀ alkenyl, C₂-C₁₀ alkynyl, optionally substituted carbocycle and optionally substituted heterocycle;

each R³⁰ is independently selected from hydrogen, optionally substituted C₁-C₆ alkyl, optionally substituted C₂-C₆ alkenyl, optionally substituted C₂-C₆ alkynyl, optionally substituted carbocycle and optionally substituted heterocycle; and

n is selected from 0 or 1;

wherein the near vision impairment is not cataract.
2. The method of embodiment 1, wherein n is 0.
3. The method of embodiment 1 wherein n is 1.
4. The method of any one of embodiments 1 to 3, wherein R¹, R², R³, R⁴, R⁶, R⁸, R⁹, R¹¹, R¹², R¹³, R¹⁴, R¹⁵, R¹⁶, and R¹⁷ are independently selected from hydrogen, halogen, —OR³⁰, SR³⁰, —OSO₃R³⁰, —OPO₃R³⁰, —N(R³¹)₂, —C(O)R³⁰, —C(O)OR³⁰, —OC(O)R³⁰, —NO₂, —CN, and optionally substituted C₁-C₁₀ alkyl.
5. The method of embodiment 4, wherein R¹, R², R³, R⁴, R⁶, R⁸, R⁹, R¹¹, R¹², R¹³, R¹⁴, R⁵, R¹⁶, and R¹⁷ are independently selected from hydrogen, halogen, —OR³⁰, —NO₂, —CN, and optionally substituted C₁-C₁₀ alkyl.
6. The method of any one of embodiments 1 to 5, wherein R⁵, R⁷, R¹⁰, R¹⁸, R¹⁹ and R²⁰ are independently selected from hydrogen, halogen, —OR³⁰, —SR³⁰, —OSO₃R³⁰, —OPO₃R³⁰, —N(R³¹)₂, —C(O)R³⁰, —C(O)OR³⁰, —OC(O)R³⁰, —NO₂, —CN, ═O, ═S, ═N(R³¹), optionally substituted C₁-C₁₀ alkyl, optionally substituted C₂-C₁₀ alkenyl, C₂-C₁₀ alkynyl.
7. The method of any one of embodiments 1 to 6, wherein R¹ and R² are independently selected from hydrogen, halogen, —OR³⁰ and optionally substituted C₁-C₁₀ alkyl, or R¹ taken together with R² is selected from ═O, ═S, and ═N(R³¹).
8. The method of any one of embodiments 1 to 7, wherein R³ is selected from hydrogen, —OR³⁰ and optionally substituted C₁-C₁₀ alkyl.
9. The method of any one of embodiments 1 to 7, wherein R³ is absent and there is a double bond between carbons 5 and 6.
10. The method of any one of embodiments 1 to 7, wherein R² and R³ are absent and there is a double bond between carbons 4 and 5.
11. The method of any one of embodiments 1 to 11, wherein R²⁰ is selected from hydrogen, halogen, —OR³⁰, —SR³⁰, —OSO₃R³⁰, —OPO₃R³⁰, —N(R³¹)₂, —C(O)R³⁰, —C(O)OR³⁰, —OC(O)R³⁰, —NO₂, —CN, ═O, ═S, ═N(R³¹), optionally substituted C₁-C₁₀ alkyl.
12. The method of embodiment 11, wherein R²⁰ is selected from hydrogen, halogen, —OR³⁰, —SR³⁰, —OSO₃R³⁰, —OPO₃R³⁰, N(R³¹)₂, —C(O)R³⁰, —C(O)OR³⁰, —OC(O)R³⁰, —NO₂, —CN, ═O, ═S, and ═N(R³¹).
13. The method of embodiment 12, wherein R²⁰ is selected from —OR³⁰, —OSO₃R³⁰, —OPO₃R³⁰, —C(O)R³⁰, —C(O)OR³⁰, —OC(O)R³⁰, ═O, and ═S.
14. The method of any one of embodiments 1 to 13, wherein R⁴ is selected from hydrogen, halogen, —OR³⁰ and optionally substituted C₁-C₁₀ alkyl.
15. The method of embodiment 14, wherein R⁴ is selected from hydrogen and —OR³⁰.
16. The method of any one of embodiments 1 to 15, wherein R⁵ is selected from hydrogen, halogen, —OR³⁰ and optionally substituted C₁-C₁₀ alkyl.
17. The method of embodiment 16, wherein R⁵ is selected from hydrogen, halogen and C₁-C₁₀ alkyl.
18. The method of any one of embodiments 1 to 17, wherein R⁶ is selected from hydrogen, halogen, —OR³⁰ and optionally substituted C₁-C₁₀ alkyl.
19. The method of embodiment 18, wherein R⁶ is selected from hydrogen, halogen and C₁-C₁₀ alkyl.
20. The method of any one of embodiments 1 to 19, wherein R⁷ is selected from hydrogen, halogen, —OR³⁰, ═O, and optionally substituted C₁-C₁₀ alkyl.
21. The method of embodiment 20, wherein R⁷ is selected from hydrogen, halogen and C₁-C₁₀ alkyl.
22. The method of any one of embodiments 1 to 21, wherein R⁸ and R⁹ are independently selected from hydrogen, halogen, —OR³⁰ and optionally substituted C₁-C₁₀ alkyl.
23. The method of embodiment 22, wherein R⁸ and R⁹ are independently selected from hydrogen, halogen, and C₁-C₁₀ alkyl.
24. The method of any one of embodiments 1 to 21, wherein R⁹ and R¹⁰ taken together with the atoms to which they are attached form an optionally substituted carbocycle or optionally substituted heterocycle.
25. The method of embodiment 24, wherein R⁹ and R¹⁰ taken together with the atoms to which they are attached form an optionally substituted carbocycle.
26. The method of any one of embodiments 1 to 23, wherein R¹⁰ is selected from —OR³⁰, ═O, ═S, ═N(R³¹), optionally substituted C₁-C₁₀ alkyl, optionally substituted C₂-C₁₀ alkenyl, and optionally substituted C₂-C₁₀ alkynyl.
27. The method of embodiment 26, wherein R¹⁰ is —OR³⁰ or ═O.
28. The method of embodiment 26, wherein R¹⁰ is optionally substituted C₁-C₁₀ alkyl or optionally substituted C₂-C₁₀ alkenyl.
29. The method of embodiment 28, wherein R¹⁰ is substituted with one or more substituents independently selected from: halogen, —OR³⁰, —SR³⁰, —OSO₃R³⁰, —OPO₃R³⁰, N(R³¹)₂, —C(O)R³⁰, —C(O)OR³⁰, —OC(O)R³⁰, —NO₂, —CN, ═O, ═S, and ═N(R³¹).
30. The method of embodiment 29, wherein R¹⁰ is substituted with one or more substituents independently selected from: halogen, —OR³⁰, —SR³⁰, —N(R³¹)₂, —C(O)R³⁰, —C(O)OR³⁰, —OC(O)R³⁰, —NO₂, —CN, ═O, ═S, and ═N(R³¹).
31. The method of embodiment 29, wherein R¹⁰ is C₁-C₁₀ alkyl substituted with one or more substituents selected from halogen and —OR³⁰.
32. The method of embodiment 29, wherein R¹⁰ is C₂-C₁₀ alkenyl optionally substituted with one or more substituents selected from halogen and —OR³⁰.
33. The method of any one of embodiments 1 to 32, wherein R¹¹ is selected from hydrogen, —OR³⁰ and optionally substituted C₁-C₁₀ alkyl.
34. The method of embodiment 33, wherein R¹¹ is hydrogen or C₁-C₁₀ alkyl.
35. The method of any one of embodiments 1 to 32, wherein R¹¹ is absent and there is a double bond between carbons 12 and 13.
36. The method of any one of embodiments 1 to 35, wherein R¹² is selected from hydrogen, halogen, —OR³⁰ and optionally substituted C₁-C₁₀ alkyl.
37. The method of embodiment 36, wherein R¹² is selected from hydrogen and —OR³⁰.
38. The method of any one of embodiments 1 to 37, wherein R¹³ and R¹⁴ are independently selected from hydrogen, halogen, —OR³⁰ and optionally substituted C₁-C₁₀ alkyl, or R¹³ taken together with R¹⁴ is selected from ═O, ═S, and ═N(R³¹).
39. The method of embodiment 38, wherein R¹³ and R¹⁴ are independently selected from hydrogen, halogen, —OR³⁰ and optionally substituted C₁-C₁₀ alkyl.
40. The method of embodiment 38, wherein R¹³ taken together with R¹⁴ is ═O.
41. The method of any one of embodiments 1 to 40, wherein R¹⁵ is selected from hydrogen and optionally substituted C₁-C₁₀ alkyl.
42. The method of embodiment 41, wherein R¹⁵ is methyl.
43. The method of any one of embodiments 1 to 42, wherein R¹⁶ and R¹⁷ are each hydrogen.
44. The method of any one of embodiments 1 to 42, wherein R¹⁶ and R¹⁷ are absent and there is a double bond between carbons 8 and 9.
45. The method of any one of embodiments 1 to 44, wherein R¹⁸ and R¹⁹ are independently selected from hydrogen, halogen, —OR³⁰, ═O, and optionally substituted C₁-C₁₀ alkyl.
46. The method of embodiment 45, wherein R¹⁸ and R¹⁹ are each hydrogen.
47. The method of any one of embodiments 1 to 46, wherein the compound of Formula (III) is represented by Formula (IIIA):

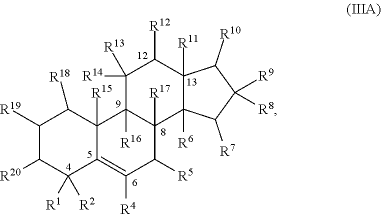

or a salt thereof.
48. The method of embodiment 47, wherein the compound of Formula (IIIA) is represented by the Formula (IIIB):

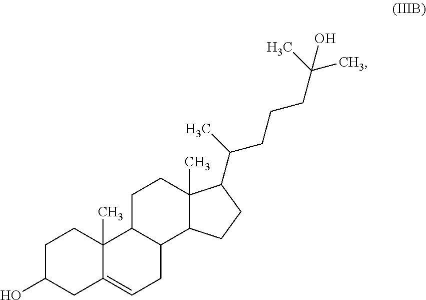 [SMILES: CC(CCCC(C)(C)O)C1CCC2C3CC=C4CC(O)CCC4(C)C3CCC12C]

or a salt thereof.
49. The method of embodiment 48, wherein the compound of Formula (IIIA) is represented by the Formula (IIIC):

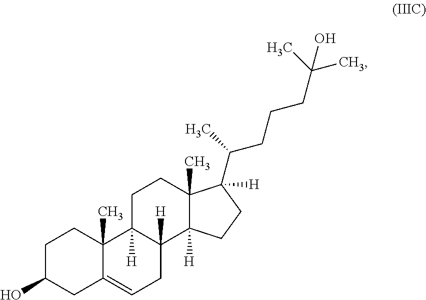

or a salt thereof.
50. The method of any one of embodiments 1 to 46, wherein the compound of Formula (III) is represented by Formula (IIID):

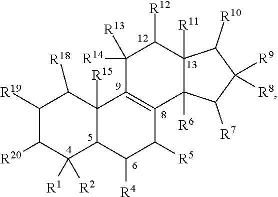

or a salt thereof.
51. The method of any one of embodiments 1 to 50, wherein said near vision impairment first occurs when the subject is 25 years old or older.
52. The method of embodiment 51, wherein said near vision impairment first occurs when the subject is 35 years old or older.
53. The method of any one of embodiments 1 to 52, wherein said near vision impairment is diagnosed by one or more of the following tests: habitual distance visual acuity, corrected near visual acuity, refractive error, optical power, Jaeger test, LogMAR scale, ETDRS scale, and accommodative amplitude of the lens.
54. The method of embodiment 53, wherein said near vision impairment comprises a pre-treatment near visual acuity value of 0.8 or less as determined by the following visual acuity test:
a. cover one eye of a subject, place the eye chart of Example 1 approximately 16 inches from the eye of said subject,
b. determine smallest size of character that subject is able to read missing no more than one character, and
c. correlate said character size with a pre-treatment visual acuity value.
55. The method of embodiment 54, wherein said near vision impairment comprises a near visual acuity of 0.6 or less.
56. The method of embodiment 55, wherein said near vision impairment comprises a near visual acuity of 0.4 or less.
57. The method of embodiment 53, wherein said near vision impairment comprises a score of J2 or higher on the Jaeger scale of the Jaeger test.
58. The method of embodiment 57, wherein said near vision impairment comprises a score of J3 or higher on the Jaeger scale of the Jaeger test.
59. The method of embodiment 58, wherein said near vision impairment comprises a score of J4 or higher on the Jaeger scale of the Jaeger test.
60. The method of embodiment 59, wherein said near vision impairment comprises a score of J5 or higher on the Jaeger scale.
61. The method of embodiment 60, wherein said near vision impairment comprises a score of J6 or higher on the Jaeger scale.
62. The method of embodiment 61, wherein said near vision impairment is determined by a score of J8 or higher on the Jaeger scale.
63. The method of embodiment 53, wherein said refractive error is assessed by one or more of the following: a retinoscope, an automated refractor, a Shack-Hartmann wavefront sensor or a pinhole occluder.
64. The method of any one of embodiments 1 to 63, wherein said treating or preventing near vision impairment of said subject comprises an improvement in one or more of visual acuity, optical power, accommodative amplitude of the lens, Jaeger scale score, LogMAR scale score, ETDRS scale, reading speed, and refractive error.
65. The method of embodiment 64, wherein said treating or preventing near vision impairment of said subject comprises an improvement in near visual acuity relative to a pre-treatment near visual acuity value.
66. The method of embodiment 65, wherein said improvement in near visual acuity is equivalent to at least 0.1 on a LogMAR scale.
67. The method of embodiment 66, wherein said improvement in near visual acuity is 0.2 or greater relative to said pre-treatment visual acuity value.
68. The method of any one of embodiments 1 to 67, wherein said treatment of near vision impairment is about equivalent to treatment with glasses or contact lenses having power of about +0.5 D or higher.
69. The method of embodiment 68, wherein said treatment of near vision impairment is about equivalent to treatment with glasses or contact lenses having power of about +1 D or higher.
70. The method of embodiment 69, wherein said treatment of near vision impairment is about equivalent to treatment with glasses or contact lenses having power of about +2D or higher.
71. The method of any one of embodiments 1 to 70, wherein prior to said administering, the subject exhibits one or more of the following symptoms: decreased focusing ability for near objects, eyestrain, difficulty reading fine print, fatigue while reading or looking at an illuminated screen, difficulty seeing clearly up close, seeing less contrast when reading print, need for brighter and more direct light for reading, and headaches when using near vision.
72. The method of anyone of embodiments 1 to 71, wherein said near vision impairment is presbyopia.
73. A method for increasing the optical power of a lens of a subject, comprising administering to a subject in need thereof a compound of Formula (III):

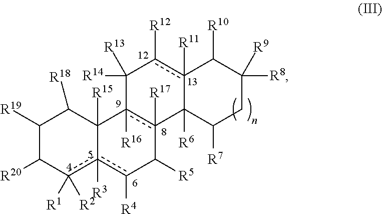

or a salt thereof, wherein:
R¹, R², R³, R⁴, R⁶, R⁸, R⁹, R¹¹, R¹², R¹³, R¹⁴, R¹⁵, R¹⁶, and R¹⁷ are independently selected from hydrogen, halogen, —OR³⁰, —SR³⁰, —OSO₃R³⁰, —OPO₃R³⁰, —N(R³¹)₂, —C(O)R³⁰, —C(O)OR³⁰, —OC(O)R³⁰, —NO₂, —CN, optionally substituted C₁-C₁₀ alkyl, optionally substituted C₂-C₁₀ alkenyl, optionally substituted C₂-C₁₀ alkynyl, optionally substituted carbocycle and optionally substituted heterocycle; R¹ taken together with R² is further selected from ═O, ═S, and ═N(R³¹); R⁸ taken together with R⁹ is further selected from ═O, ═S, and ═N(R³¹); R¹³ taken together with R¹⁴ is further selected from ═O, ═S, and ═N(R³¹); R⁹ and R¹⁰ taken together with the atoms to which they are attached may further form an optionally substituted carbocycle or optionally substituted heterocycle; and wherein R³ is absent when there is a double bond between carbons 5 and 6, R¹⁶ and R¹⁷ are absent when there is a double bond between carbons 8 and 9, R¹¹ is absent when there is a double bond between carbons 12 and 13; and R² and R³ are absent and there is a single bond between carbons 5 and 6 when there is a double bond between carbons 4 and 5;
R⁵, R⁷, R¹⁰, R¹⁸, R¹⁹ and R²⁰ are independently selected from hydrogen, halogen, —OR³⁰, —SR³⁰, —OSO₃R³⁰, —OPO₃R³⁰, —N(R³¹)₂, —C(O)R³⁰, —C(O)OR³⁰, —OC(O)R³⁰, —NO₂, —CN, ═O, ═S, ═N(R³¹), optionally substituted C₁-C₁₀ alkyl, optionally substituted C₂-C₁₀ alkenyl, C₂-C₁₀ alkynyl, optionally substituted carbocycle and optionally substituted heterocycle;
each R³¹ is independently selected from hydrogen, —OR³⁰, —SR³⁰, —S(O)R³⁰, —S(O)₂R³⁰, C(O)R³⁰, —C(O)OR³⁰, optionally substituted C₁-C₁₀ alkyl, optionally substituted C₂-C₁₀ alkenyl, C₂-C₁₀ alkynyl, optionally substituted carbocycle and optionally substituted heterocycle;
each R³⁰ is independently selected from hydrogen, optionally substituted C₁-C₆ alkyl, optionally substituted C₂-C₆ alkenyl, optionally substituted C₂-C₆ alkynyl, optionally substituted carbocycle and optionally substituted heterocycle; and
n is selected from 0 or 1,
wherein the lens does not have cataract.
74. The method of embodiment 73, wherein the lens has a pre-treatment optical power of less than 20 diopters.
75. The method of embodiment 74, wherein the lens has a pre-treatment optical power of less than 15 diopters.
76. The method of any one of embodiments 73 to 75, wherein increasing the optical power of the lens comprises improving optical power by at least 0.1 diopters relative to a pre-treatment optical power.
77. The method of embodiment 76, wherein increasing the optical power of the lens comprises improving optical power by at least 1 diopter relative to a pre-treatment optical power.
78. The method of embodiment 77, wherein increasing the optical power of the lens comprises improving optical power by at least 5 diopters relative to a pre-treatment optical power.
79. The method of any one of embodiments 1 to 72, wherein the near vision impairment is not hyperopia.
80. The method of any one of embodiments 1 to 79, wherein said administering comprises administering for four weeks or more.
81. The method of any one of embodiments 1 to 80, wherein said administering comprises administration topically, subconjunctivally, retrobulbarly, periocularly, subretinally, suprachoroidally, or intraocularly.
82. The method of any one of embodiments 1 to 81, wherein said method further comprises administering an additional therapeutic agent.
83. The method of embodiments 82, wherein said additional therapeutic agent is lipoic acid.

What is claimed is:

1. A pharmaceutical formulation comprising from about 0.05 wt % to about 5 wt % of a compound represented by formula (IIIC): 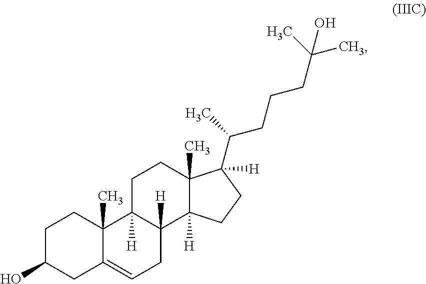
or a salt thereof, and one or more pharmaceutically acceptable excipients.

2. The pharmaceutical formulation of claim 1, wherein the formulation comprises from about 0.1 wt % to about 4 wt % of a compound or salt of formula (IIIC).

3. The pharmaceutical formulation of claim 2, wherein the formulation comprises from about 0.5 wt % to about 4 wt % of a compound or salt of formula (IIIC).

4. The pharmaceutical formulation of claim 1, wherein the formulation comprises from about 2 wt % to about 4 wt % of a compound or salt of formula (IIIC).

5. The pharmaceutical formulation of any one of claims 1 to 4, wherein the compound or salt of formula (IIIC) is in the form of particles and wherein the particles have an average largest diameter selected from about 1 nm to about 1 μm.

6. The pharmaceutical formulation of claim 5, wherein the particles of a compound or salt of formula (IIIC) have an average diameter selected from about 1 nm to about 200 nm.

7. The pharmaceutical formulation of claim 5, wherein the particles of a compound or salt of formula (IIIC) have an average diameter selected from about 400 nm to about 600 nm.

8. The pharmaceutical formulation of claim 7, wherein the particles of a compound or salt of formula (IIIC) have an average diameter selected from about 450 to about 550 nm.

9. The pharmaceutical formulation of any one of claims 1 to 4, wherein greater than 80% of the particles have an average largest diameter selected from about 450 nm to about 550 nm.

10. The pharmaceutical formulation of any one of claims 1 to 9, wherein the formulation comprises at least about 90 wt % water.

11. The pharmaceutical formulation of any one of claims 1 to 10, wherein the formulation comprises an agent that increases the viscosity of the formulation.

12. The pharmaceutical formulation of claim 11, wherein the agent that increases the viscosity of the formulation is selected from carboxymethyl cellulose (CMC), hydroxyethyl cellulose, polyethylene glycol (PEG), sodium carboxymethyl cellulose, hydroxypropyl methyl cellulose (HPMC), sorbitol, gellan gum (high or low acyl), xanthan gum, dextran, guar gum, locust bean gum, sodium alginate, agar, gelatin, chitosan, pectin, alginates, xyloglucan, polyvinyl alcohol, polyvinyl pyrrolidone, carrageenan and combinations thereof.

13. The pharmaceutical formulation of claim 12, wherein the agent that increases the viscosity of the formulation is gellan gum.

14. The pharmaceutical formulation of any one of claims 1 to 13, wherein the formulation has a viscosity of about 0.005 Pa·s to about 0.030 Pa·s.

15. The pharmaceutical formulation of any one of claims 1 to 14, wherein the formulation comprises an agent for adjusting the pH of the formulation.

16. The pharmaceutical formulation of claim 15, wherein the agent for adjusting the pH of the formulation is selected from hydrochloric acid, boric acid, sodium hydroxide and potassium hydroxide.

17. The pharmaceutical formulation of claim 16, wherein the agent for adjusting the pH of the formulation is boric acid.

18. The pharmaceutical formulation of any one of claims 1 to 17, wherein the formulation has a pH selected from about 5 to about 9.

19. The pharmaceutical formulation of claim 18, wherein the formulation has a pH selected from about 7 to about 8.

20. The pharmaceutical formulation of claim 19, wherein the formulation has a pH of about 7.4.

21. The pharmaceutical formulation of any one of claims 1 to 20, wherein the formulation comprises an agent for adjusting the osmolarity of the formulation.

22. The pharmaceutical formulation of claim 21, wherein the agent for adjusting the osmolarity of the formulation is mannitol.

23. The pharmaceutical formulation of any one of claims 1 to 22, wherein the formulation comprises a buffering agent.

24. The pharmaceutical formulation of claim 23, wherein the buffering agent is selected from tromethamine, potassium phosphate, sodium phosphate, saline sodium citrate buffer (SSC), acetate, saline, physiological saline, phosphate buffer saline (PBS), 4-2-hydroxyethyl-1-piperazineethanesulfonic acid buffer (HEPES), 3-(N-morpholino)propanesulfonic acid buffer (MOPS), and piperazine-N,N′-bis(2-ethanesulfonic acid) buffer (PIPES), sodium acetate-boric acid stock solution, boric acid-sodium carbonate with sodium chloride solution, boric acid-sodium borate buffer, sodium and potassium phosphate buffers, boric acid-sodium carbonate with potassium chloride, or combinations thereof.

25. The pharmaceutical formulation of claim 23 or 24, wherein the formulation comprises from about 0.1 wt % to about 4 wt % of a buffering agent.

26. The pharmaceutical formulation of any one of claims 1 to 25, wherein the formulation comprises a dispersion agent.

27. The pharmaceutical formulation of claim 26, wherein the formulation comprises from about 0.01 wt % to about 1 wt % of a dispersion agent.

28. The pharmaceutical formulation of any one of claims 1 to 27, wherein the formulation comprises a preservative agent.

29. The pharmaceutical formulation of claim 28, wherein the preservative agent is selected from benzalkonium chloride, ethylenediaminetetraacetic acid (EDTA), chlorobutanol, phenylmercuric acetate, phenylmercuric nitrate, chlorhexidine acetate, thimerosal, and benzethonium chloride.

30. The pharmaceutical formulation of claim 28 or 29, wherein the formulation comprises from about 0.001 wt % to about 0.1 wt % of a preservative agent.

31. The pharmaceutical formulation of any one of claims 1 to 30, wherein the formulation does not include a preservative agent.

32. A method for treating an ophthalmic disease comprising administering a pharmaceutical formulation of any one of claims 1 to 31 to the eye of a subject in need thereof.

33. The method of claim 32, wherein the pharmaceutical formulation is administered topically, by intravitreal injection or intracameral injection.

34. The method of claim 33, wherein the pharmaceutical formulation is administered by intravitreal injection or intracameral injection.

35. The method of claim 33 or 34, wherein the pharmaceutical formulation is administered in one or more doses wherein each dose is selected from about 60 μL to about 120 μL.

36. The method of claim 35, wherein the pharmaceutical formulation is administered in one or more doses wherein each dose is from about 80 μL to about 110 μL.

37. The method of claims 35 or 36, wherein a dose of the pharmaceutical formulation is administered once monthly, once every six weeks, once every two months, once every six months, or once yearly.

38. The method of claim 35 or 36, wherein a dose of the pharmaceutical formulation is administered once a month for three consecutive months followed by a dosing holiday of one month, two months, three months, four months, five months, six months, nine months or a year.

39. The method of claims 35 or 36, wherein a dose of the pharmaceutical formulation is administered once a month for two consecutive months followed by a dosing holiday of one month, two months, three months, four months, five months, six months, nine months or a year.

40. The method of claim 33, wherein the pharmaceutical formulation is administered topically.

41. The method of any one of claims 32 to 40, wherein the ophthalmic disease is cataract.

42. The method of any one of claims 32 to 40, wherein the ophthalmic disease is presbyopia.

43. A method of treating or preventing a near vision disorder of a subject, comprising administering to a subject in need thereof a compound of Formula (III): 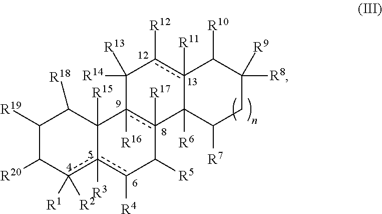
or a salt thereof, wherein:
R¹, R², R³, R⁴, R⁶, R⁸, R⁹, R¹¹, R¹², R¹³, R¹⁴, R¹⁵, R¹⁶, and R¹⁷ are independently selected from hydrogen, halogen, —OR³⁰, —SR³⁰, —OSO₃R³, —OPO₃R³, —N(R³¹)₂, —C(O)R³⁰, —C(O)OR³⁰, —OC(O)R³⁰, —NO₂, —CN, optionally substituted C₁-C₁₀ alkyl, optionally substituted C₂-C₁₀ alkenyl, optionally substituted C₂-C₁₀ alkynyl, optionally substituted carbocycle and optionally substituted heterocycle; R¹ taken together with R² is further selected from ═O, ═S, and ═N(R³¹); R⁸ taken together with R⁹ is further selected from ═O, ═S, and ═N(R³¹); R¹³ taken together with R¹⁴ is further selected from ═O, ═S, and ═N(R³¹); R⁹ and R¹⁰ taken together with the atoms to which they are attached may further form an optionally substituted carbocycle or optionally substituted heterocycle; and wherein R³ is absent when there is a double bond between carbons 5 and 6, R¹⁶ and R¹⁷ are absent when there is a double bond between carbons 8 and 9, R¹¹ is absent when there is a double bond between carbons 12 and 13; and R² and R³ are absent and there is a single bond between carbons 5 and 6 when there is a double bond between carbons 4 and 5;
R⁵, R⁷, R¹⁰, R¹⁸, R¹⁹ and R²⁰ are independently selected from hydrogen, halogen, —OR³⁰, —SR³⁰, —OSO₃R³⁰, —OPO₃R³⁰, N(R³¹)₂, —C(O)R³⁰, —C(O)OR³⁰, —OC(O)R³⁰, —NO₂, —CN, ═O, ═S, ═N(R³¹), optionally substituted C₁-C₁₀ alkyl, optionally substituted C₂-C₁₀ alkenyl, C₂-C₁₀ alkynyl, optionally substituted carbocycle and optionally substituted heterocycle;
each R³¹ is independently selected from hydrogen, —OR³⁰, —SR³⁰, —S(O)R³⁰, —S(O)₂R³⁰, —C(O)R³⁰, —C(O)OR³⁰, optionally substituted C₁-C₁₀ alkyl, optionally substituted C₂-C₁₀ alkenyl, C₂-C₁₀ alkynyl, optionally substituted carbocycle and optionally substituted heterocycle;
each R³⁰ is independently selected from hydrogen, optionally substituted C₁-C₆ alkyl, optionally substituted C₂-C₆ alkenyl, optionally substituted C₂-C₆ alkynyl, optionally substituted carbocycle and optionally substituted heterocycle; and
n is selected from 0 or 1
wherein the near vision disorder is not cataract.
